SEQUENCE LISTING (265 sequences)

SEQ ID NO: 1 - protein, 8 aa, Homo sapiens
GYSFTSYF

SEQ ID NO: 2 - protein, 8 aa, Homo sapiens
INPAGGNT

SEQ ID NO: 3 - protein, 13 aa, Homo sapiens
ARDKDYGEGAFDL

SEQ ID NO: 4 - protein, 10 aa, Homo sapiens
GGSISSGGYS

SEQ ID NO: 5 - protein, 7 aa, Homo sapiens
IYHSGST

SEQ ID NO: 6 - protein, 19 aa, Homo sapiens
ARELGYCSSTSCPWGAFDI

SEQ ID NO: 7 - protein, 8 aa, Homo sapiens
GFTFDDYG

SEQ ID NO: 8 - protein, 8 aa, Homo sapiens
INWNGGST

SEQ ID NO: 9 - protein, 16 aa, Homo sapiens
ARVNYDSSGLRGAFDI

SEQ ID NO: 10 - protein, 8 aa, Homo sapiens
GFTFSSYA

SEQ ID NO: 11 - protein, 8 aa, Homo sapiens
ISYDGSNK

SEQ ID NO: 12 - protein, 17 aa, Homo sapiens
ARESGVSGDYYYYGMDV

SEQ ID NO: 13 - protein, 8 aa, Homo sapiens
GFTFDDYG

SEQ ID NO: 14 - protein, 8 aa, Homo sapiens
INWNGGST

SEQ ID NO: 15 - protein, 16 aa, Homo sapiens
ARVNYDSSGLRGAFDI

SEQ ID NO: 16 - protein, 8 aa, Homo sapiens
GFTFDDYA

SEQ ID NO: 17 - protein, 8 aa, Homo sapiens
ISWNSGSI

SEQ ID NO: 18 - protein, 14 aa, Homo sapiens
AKDITSSGWYWFDY

SEQ ID NO: 19 - protein, 8 aa, Homo sapiens
GFTFDDYG

SEQ ID NO: 20 - protein, 8 aa, Homo sapiens
INWNGGST

SEQ ID NO: 21 - protein, 16 aa, Homo sapiens
ARVNYDSSGLRGAFDI

SEQ ID NO: 22 - protein, 8 aa, Homo sapiens
GFTFSSYS

SEQ ID NO: 23 - protein, 8 aa, Homo sapiens
ISSSSSYI

SEQ ID NO: 24 - protein, 13 aa, Homo sapiens
ARDIALPYYGMDV

SEQ ID NO: 25 - protein, 8 aa, Homo sapiens
GGTFSSYA

SEQ ID NO: 26 - protein, 8 aa, Homo sapiens
IIPISGTA

SEQ ID NO: 27 - protein, 21 aa, Homo sapiens
GRGDYCNGGSCSYTNYYGMDV

SEQ ID NO: 28 - protein, 8 aa, Homo sapiens
GYTFTSYY

SEQ ID NO: 29 - protein, 8 aa, Homo sapiens
INPSGGST

SEQ ID NO: 30 - protein, 17 aa, Homo sapiens
ARDKAHDSSGYYDAFDI

SEQ ID NO: 31 - protein, 8 aa, Homo sapiens
GFTFSSYA

SEQ ID NO: 32 - protein, 8 aa, Homo sapiens
ISGSGGST

SEQ ID NO: 33 - protein, 21 aa, Homo sapiens
ANYHGPDISLTYYDFWSGYPV

SEQ ID NO: 34 - protein, 8 aa, Homo sapiens
GGTFSSYA

SEQ ID NO: 35 - protein, 8 aa, Homo sapiens
IIPIVGTA

SEQ ID NO: 36 - protein, 21 aa, Homo sapiens
ARGDYCSGGSCSYTNYYGMDV

SEQ ID NO: 37 - protein, 8 aa, Homo sapiens
GFTFDDYA

SEQ ID NO: 38 - protein, 8 aa, Homo sapiens
ISWNSGSI

SEQ ID NO: 39 - protein, 13 aa, Homo sapiens
AKDIGVYYYGMDV

SEQ ID NO: 40 - protein, 8 aa, Homo sapiens
GFTFDDYG

SEQ ID NO: 41 - protein, 8 aa, Homo sapiens
INWNGGST

SEQ ID NO: 42 - protein, 16 aa, Homo sapiens
ARVNYDSSGLRGAFDI

SEQ ID NO: 43 - protein, 9 aa, Homo sapiens
GGSMGSSNW

SEQ ID NO: 44 - protein, 7 aa, Homo sapiens
VYHTGST

SEQ ID NO: 45 - protein, 12 aa, Homo sapiens
ARDLTVTGSFDI

SEQ ID NO: 46 - protein, 8 aa, Homo sapiens
GYTFTGYY

SEQ ID NO: 47 - protein, 8 aa, Homo sapiens
INPNSGGT

SEQ ID NO: 48 - protein, 16 aa, Homo sapiens
ARVKSYYYDSNDAFDI

SEQ ID NO: 49 - protein, 8 aa, Homo sapiens
GFTFDDYA

SEQ ID NO: 50 - protein, 8 aa, Homo sapiens
ISWNSGSI

SEQ ID NO: 51 - protein, 13 aa, Homo sapiens
AKDIGVYYYGMDV

SEQ ID NO: 52 - protein, 8 aa, Homo sapiens
GFTFDDYA

SEQ ID NO: 53 - protein, 8 aa, Homo sapiens
ISWNSGSI

SEQ ID NO: 54 - protein, 14 aa, Homo sapiens
AKGLGVLAYYGMDI

SEQ ID NO: 55 - protein, 8 aa, Homo sapiens
GYTFTGYY

SEQ ID NO: 56 - protein, 8 aa, Homo sapiens
INPNSGGT

SEQ ID NO: 57 - protein, 19 aa, Homo sapiens
ARDKGAWSGSYPGYNWFDP

SEQ ID NO: 58 - protein, 8 aa, Homo sapiens
GYTFTSYY

SEQ ID NO: 59 - protein, 8 aa, Homo sapiens
INPSGGST

SEQ ID NO: 60 - protein, 11 aa, Homo sapiens
ARGTTYDAFDI

SEQ ID NO: 61 - protein, 8 aa, Homo sapiens
GYTFTTSY

SEQ ID NO: 62 - protein, 8 aa, Homo sapiens
VNPNDGST

SEQ ID NO: 63 - protein, 20 aa, Homo sapiens
AKDIRDYVWGTYRYESAFDI

SEQ ID NO: 64 - protein, 8 aa, Homo sapiens
GGTFSSYA

SEQ ID NO: 65 - protein, 8 aa, Homo sapiens
IIPISGTA

SEQ ID NO: 66 - protein, 21 aa, Homo sapiens
ARGDYCNGGSCSYTNYYGMDV

SEQ ID NO: 67 - protein, 8 aa, Homo sapiens
GYSFSSHG

SEQ ID NO: 68 - protein, 8 aa, Homo sapiens
ISAYNGNT

SEQ ID NO: 69 - protein, 17 aa, Homo sapiens
ARDQDEANWNNNYYFDY

SEQ ID NO: 70 - protein, 8 aa, Homo sapiens
GGTFSSYA

SEQ ID NO: 71 - protein, 8 aa, Homo sapiens
IIPIFGTA

SEQ ID NO: 72 - protein, 15 aa, Homo sapiens
AREATPFMILGAFDI

SEQ ID NO: 73 - protein, 8 aa, Homo sapiens
GYSFSTYW

SEQ ID NO: 74 - protein, 8 aa, Homo sapiens
IYPGDSDT

SEQ ID NO: 75 - protein, 9 aa, Homo sapiens
ARGPYGMDV

SEQ ID NO: 76 - protein, 8 aa, Homo sapiens
GGTFSSYA

SEQ ID NO: 77 - protein, 8 aa, Homo sapiens
IIPIFGTA

SEQ ID NO: 78 - protein, 14 aa, Homo sapiens
ASSYYGSGTTWFDP

SEQ ID NO: 79 - protein, 9 aa, Homo sapiens
SSNIGAGYD

SEQ ID NO: 80 - protein, 11 aa, Homo sapiens
QSYDSSLSGWV

SEQ ID NO: 81 - protein, 8 aa, Homo sapiens
SSNIGNNY

SEQ ID NO: 82 - protein, 11 aa, Homo sapiens
GTWDSSLSAVV

SEQ ID NO: 83 - protein, 6 aa, Homo sapiens
QDISNY

SEQ ID NO: 84 - protein, 9 aa, Homo sapiens
QQYDNLPIT

SEQ ID NO: 85 - protein, 6 aa, Homo sapiens
QDIKNY

SEQ ID NO: 86 - protein, 9 aa, Homo sapiens
QHYDSLPIT

SEQ ID NO: 87 - protein, 9 aa, Homo sapiens
CDRQDISNY

SEQ ID NO: 88 - protein, 8 aa, Homo sapiens
QQYDNLPT

SEQ ID NO: 89 - protein, 8 aa, Homo sapiens
NSNIGSNA

SEQ ID NO: 90 - protein, 11 aa, Homo sapiens
AAWDDSLNGPV

SEQ ID NO: 91 - protein, 6 aa, Homo sapiens
QYISNS

SEQ ID NO: 92 - protein, 9 aa, Homo sapiens
QQYHNLPPT

SEQ ID NO: 93 - protein, 8 aa, Homo sapiens
SSNIGSSP

SEQ ID NO: 94 - protein, 11 aa, Homo sapiens
AAWDDSLNAYV

SEQ ID NO: 95 - protein, 8 aa, Homo sapiens
SANIGSNS

SEQ ID NO: 96 - protein, 11 aa, Homo sapiens
AAWDDSLKGPV

SEQ ID NO: 97 - protein, 8 aa, Homo sp.
GSNIGSNT

SEQ ID NO: 98 - protein, 11 aa, Homo sapiens
ATWDDSLNDYV

SEQ ID NO: 99 - protein, 8 aa, Homo sapiens
SGSIANNH

SEQ ID NO: 100 - protein, 9 aa, Homo sapiens
QSYDDNNWV

SEQ ID NO: 101 - protein, 8 aa, Homo sapiens
SSNIGSNT

SEQ ID NO: 102 - protein, 11 aa, Homo sapiens
AAWDDSLNGYV

SEQ ID NO: 103 - protein, 6 aa, Homo sapiens
QSVGTY

SEQ ID NO: 104 - protein, 9 aa, Homo sapiens
QQSYSTPIT

SEQ ID NO: 105 - protein, 6 aa, Homo sapiens
QDISNY

SEQ ID NO: 106 - protein, 8 aa, Homo sapiens
QQYDNLPT

SEQ ID NO: 107 - protein, 9 aa, Homo sapiens
SSDVGAYNY

SEQ ID NO: 108 - protein, 10 aa, Homo sapiens
FSYAGNYNWV

SEQ ID NO: 109 - protein, 11 aa, Homo sapiens
QSLLHSNGYNY

SEQ ID NO: 110 - protein, 9 aa, Homo sapiens
MQALQTPHT

SEQ ID NO: 111 - protein, 8 aa, Homo sapiens
SSNIGSSP

SEQ ID NO: 112 - protein, 11 aa, Homo sapiens
AAWDDSLNAYV

SEQ ID NO: 113 - protein, 6 aa, Homo sapiens
QGISSW

SEQ ID NO: 114 - protein, 9 aa, Homo sapiens
QQANSFPLT

SEQ ID NO: 115 - protein, 8 aa, Homo sapiens
SSTIGTNP

SEQ ID NO: 116 - protein, 11 aa, Homo sapiens
ATLDDSLNGYV

SEQ ID NO: 117 - protein, 8 aa, Homo sapiens
SSNIGSNT

SEQ ID NO: 118 - protein, 12 aa, Homo sapiens
AAWDDSLNGLYV

SEQ ID NO: 119 - protein, 6 aa, Homo sapiens
KLGDKY

SEQ ID NO: 120 - protein, 11 aa, Homo sapiens
QTWDSSTASYV

SEQ ID NO: 121 - protein, 6 aa, Homo sapiens
NIRSQN

SEQ ID NO: 122 - protein, 11 aa, Homo sapiens
QVWDATRDHVV

SEQ ID NO: 123 - protein, 8 aa, Homo sapiens
SGSIASKY

SEQ ID NO: 124 - protein, 10 aa, Homo sapiens
QSYDSANRVV

SEQ ID NO: 125 - protein, 8 aa, Homo sapiens
SSNIGSNT

SEQ ID NO: 126 - protein, 11 aa, Homo sapiens
AAWDDSLNGVV

SEQ ID NO: 127 - protein, 6 aa, Homo sapiens
TIRTKT

SEQ ID NO: 128 - protein, 11 aa, Homo sapiens
QMWDGGSSLVV

SEQ ID NO: 129 - protein, 6 aa, Homo sapiens
QSISSY

SEQ ID NO: 130 - protein, 9 aa, Homo sapiens
QQSYSTLRT

SEQ ID NO: 131 - protein, 120 aa, Homo sapiens
QVQLVQSGAEVKKPGASVKVSCKASGYSFTSYFIHWVRQAPGQGLEWMGIINPAGGNTNYAQRFQGRVTMTRDTSTSTVYMELSSLRSDDTAVYYCARDKDYGEGAFDLWGQGTMVSVSS

SEQ ID NO: 132 - protein, 127 aa, Homo sapiens
QVELQESGSGLVKPSQTLSLTCAVSGGSISSGGYSWSWIRQPPGKGLEWIGYIYHSGSTYYNPSLKSRVTISVDRSKNQFSLKLSSVTAADTAVYYCARELGYCSSTSCPWGAFDIWGQGTLVSVSS

SEQ ID NO: 133 - protein, 123 aa, Homo sp.
QVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARVNYDSSGLRGAFDIWGQGTMVIVSS

SEQ ID NO: 134 - protein, 124 aa, Homo sapiens
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARESGVSGDYYYYGMDVWGQGTMVTVSS

SEQ ID NO: 135 - protein, 123 aa, Homo sapiens
QVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARVNYDSSGLRGAFDIWGQGTTVTVSS

SEQ ID NO: 136 - protein, 121 aa, Homo sapiens
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDITSSGWYWFDYWGQGTLVTVSS

SEQ ID NO: 137 - protein, 123 aa, Homo sapiens
QVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARVNYDSSGLRGAFDIWGQGTMVTVSS

SEQ ID NO: 138 - protein, 120 aa, Homo sapiens
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDIALPYYGMDVWGQGTMVTVSS

SEQ ID NO: 139 - protein, 128 aa, Homo sapiens
QVQLVQSGAEVKKPGSSVKVSCKTSGGTFSSYAISWVRQAPGQGLEWMGGIIPISGTANYAQKLQGRVTITADESTSTAYMELSSLRSEDTAVYYCGRGDYCNGGSCSYTNYYGMDVWGQGTMVTVSS

SEQ ID NO: 140 - protein, 124 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDKAHDSSGYYDAFDIWGQGTTVTVSS

SEQ ID NO: 141 - protein, 128 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCANYHGPDISLTYYDFWSGYPVWGQGTLVTVSS

SEQ ID NO: 142 - protein, 128 aa, Homo sapiens
EVQLVQSGAEVKKPGSSVKVSCKASGGTFSSYAITWVRQAPGQGLEWMGGIIPIVGTANYAQKFQGRLTITADESTSTAYMELSSLRSEDTAVYYCARGDYCSGGSCSYTNYYGMDVWGQGTTVTVSS

SEQ ID NO: 143 - protein, 120 aa, Homo sapiens
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGVYYYGMDVWGQGTTVTVSS

SEQ ID NO: 144 - protein, 123 aa, Homo sapiens
QVELVESGGGVVRPGGSLRLSCAASGFTFDDYGMSWVRQAPGKGLEWVSGINWNGGSTGYADSVKGRFTISRDNAKNSLYLQMNSLRAEDTALYHCARVNYDSSGLRGAFDIWGQGTTVTVSS

SEQ ID NO: 145 - protein, 119 aa, Homo sapiens
QVELQESGPGLVQPSGTLSLTCAVSGGSMGSSNWWSWVRQPPGKGLEWIGEVYHTGSTNYNPSLRSRVTISVDKSKKQFSLELTSVTAADTAVYFCARDLTVTGSFDIWGQGTMVTVSS

SEQ ID NO: 146 - protein, 123 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARVKSYYYDSNDAFDIWGQGTMVSVSS

SEQ ID NO: 147 - protein, 120 aa, Homo sapiens
QVELVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGVYYYGMDVWGQGTTVSVSS

SEQ ID NO: 148 - protein, 121 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGLGVLAYYGMDIWGQGTMVIVSS

SEQ ID NO: 149 - protein, 126 aa, Homo sapiens
EVQLVQSGAEVKKPGASVKVSCKASGYTFTGYYMHWVRQAPGHGLEWMGWINPNSGGTNYAQKFQGRVTMTRDTSISTAYMELSGLRSDDTAVYYCARDKGAWSGSYPGYNWFDPWGQGTLVTVSS

SEQ ID NO: 150 - protein, 118 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGTTYDAFDIWGQGTTVSVSS

SEQ ID NO: 151 - protein, 127 aa, Homo sapiens
EVQLVQSGPEVRKPGASVQISCKASGYTFTTSYIHWVRQAPGQGLEWLGLVNPNDGSTTYTQNFQGRVTMTSDTSTSTVYMELSRVTYADTAVYYCAKDIRDYVWGTYRYESAFDIWGQGTTVTVSS

SEQ ID NO: 152 - protein, 128 aa, Homo sapiens
EVQLVQSGAEVKKPGSSVKVSCKTSGGTFSSYAISWVRQAPGQGLEWMGGIIPISGTANYPQKLQGRVTITADESTSTAYMELSSLRSEDTAMYYCARGDYCNGGSCSYTNYYGMDVWGQGTTVIVSS

SEQ ID NO: 153 - protein, 124 aa, Homo sapiens
QVQLVQSGAEVKKPGASVKVSCKVSGYSFSSHGISWVRQAPGQGLEWMGWISAYNGNTNYAQNLQGRVRMTTDTSTNTAHMELRSLRSDDTAVYYCARDQDEANWNNNYYFDYWGQGTLVTVSS

SEQ ID NO: 154 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREATPFMILGAFDIWGQGTMVIVSS

SEQ ID NO: 155 - protein, 116 aa, Homo sapiens
QVQLVQSGAEVKKPGESLKISCKGSGYSFSTYWIAWVRQMPGKGLEWMGIIYPGDSDTRYSPSFQGQVTISADKSISTAYLQWSSLKASDTAMYYCARGPYGMDVWGQGTTVSVSS

SEQ ID NO: 156 - protein, 121 aa, Homo sapiens
QVQLLQSGAEVKKPGSSVKVSCKASGGTFSSYAISWVRQAPGQGLEWMGGIIPIFGTANYAQKFQGRVTITADESTSTAYMELSSLRSEDTAVYYCASSYYGSGTTWFDPWGQGTLVTVSS

SEQ ID NO: 157 - protein, 111 aa, Homo sapiens
QSALTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKVTVL

SEQ ID NO: 158 - protein, 110 aa, Homo sp.
QAVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTQLTVL

SEQ ID NO: 159 - protein, 107 aa, Homo sapiens
DIVMTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 160 - protein, 107 aa, Homo sapiens
AIQLTQSPSSLSASVGDRVTITCQASQDIKNYLNWYQQRPGKAPKLLIYDASNLETGVPSRFSGGGSGTDFTFTISSLQPEDIATYYCQHYDSLPITFGQGTRLEIK

SEQ ID NO: 161 - protein, 106 aa, Homo sapiens
AIQLTQSPSSLSASVGDRVTITCQASQDISNYLNWYQQKPGKAPKLLIYDASNLETGVPSRFSGSGSGTDFTFTISSLQPEDIATYYCQQYDNLPTFGQGTRLEIK

SEQ ID NO: 162 - protein, 110 aa, Homo sapiens
QPVLTQPPSASGTPGQRVTISCSGSNSNIGSNAVNWYQQLPGTAPKLLIHSHNQRPSGVPERFSGSKSGTSASLAISGLQSEDEADYYCAAWDDSLNGPVFGGGTKLTVL

SEQ ID NO: 163 - protein, 107 aa, Homo sapiens
NIQMTQSPSSLSASVGDRVTITCQASQYISNSLNWYQQKPGKAPNLLLYDATNLEPGVPSRFSGSGSRTDFTLTISSLQPEDIATYYCQQYHNLPPTFGQGTRLEIK

SEQ ID NO: 164 - protein, 110 aa, Homo sapiens
QSVLTQPPSASGTPGQKVTISCSGSSSNIGSSPVNWYQQLPGPAPKLLIHSNNQRPSGVPDRFSGSKSGTSASLAISGLQSDDEADYYCAAWDDSLNAYVFGTGTELTVL

SEQ ID NO: 165 - protein, 110 aa, Homo sapiens
QAVVTQPPSASGTPGQRSTISCSGGSANIGSNSVHWYQQFPGTAPKLLIFNNNQRPSGVPDRFSGSKSGTSASLAISGLQSEDEADYFCAAWDDSLKGPVFGGGTKVTVL

SEQ ID NO: 166 - protein, 110 aa, Homo sapiens
QSALTQPPSASGTPGQRVTISCSGSGSNIGSNTVNWYQQLPGTAPRLLIYRNNQRPSGVPDRFSGSKSGTSASLAISGLESEDEADYFCATWDDSLNDYVFATGTQLTVL

SEQ ID NO: 167 - protein, 110 aa, Homo sapiens
QSALTQPHSVSESPGKTVTISCTRSSGSIANNHVQWYQQRPGSAPIIVIYEDNQRPSGVPDRFSGSIDSSSNSASLTLSGLKTEDEAAYYCQSYDDNNWVFGGGTKVTVL

SEQ ID NO: 168 - protein, 110 aa, Homo sapiens
QAVVTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKVTVL

SEQ ID NO: 169 - protein, 107 aa, Homo sapiens
DVQMTQSPSSLSASVGDRVTITCRAGQSVGTYLNWYQQKPGKAPKLLIYAASTLQSGVPSRFSGSGSGTDFTLTISSLQPEDFATYYCQQSYSTPITFGQGTRLEIK

SEQ ID NO: 170 - protein, 106 aa, Homo sapiens
DVQMTQSPSSLSASVGDRVTITCQASQDISNYLNWYQQKPGKAPKLLIYDASNLETGVPSRFSGSGSGTDFTFTISSLQPEDIATYYCQQYDNLPTFGQGTRLEIK

SEQ ID NO: 171 - protein, 110 aa, Homo sapiens
QAVVTQPRSVSGSPGQSVTISCTGSSSDVGAYNYVSWYQQHPGKAPKLIISDVSDWPSGVPDRFSGSKSGNTASLTISGLQADDEADYYCFSYAGNYNWVFGGGTKLTVL

SEQ ID NO: 172 - protein, 112 aa, Homo sapiens
DVVMTQSPLSLPVTPGEPASISCRSSQSLLHSNGYNYLDWYMQKPGQSPQVLIYLGSNRASGVPDRFSGSGSGTDFTLKISRVEAEDVGVYYCMQALQTPHTFGQGTTVDIK

SEQ ID NO: 173 - protein, 110 aa, Homo sapiens
QSALTQPPSASGTPGQKVTISCSGSSSNIGSSPVNWYQQFPGPAPKLLIHSNNQRPSGVPDRFSGSKSGTSASLAISGLQSDDEADYYCAAWDDSLNAYVFGTGTKVTVL

SEQ ID NO: 174 - protein, 107 aa, Homo sapiens
AIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVDIK

SEQ ID NO: 175 - protein, 110 aa, Homo sapiens
QAVVTQPPSASGTPGQRVTISCSGSSSTIGTNPVNWYRHLPGTAPKLLIYSNNQRPSGVPDRVSGSKSGTSASLAISGLQSEDEADYYCATLDDSLNGYVFGTGTKVTVL

SEQ ID NO: 176 - protein, 111 aa, Homo sapiens
QSALTQPPSASGTPGQRVTISCSGSSSNIGSNTVNWYQQLPGTAPKLLIYSNNQRPSGVPDRFSGSKSGTSASLAISGLQSEDEADYYCAAWDDSLNGLYVFGTGTKVTVL

SEQ ID NO: 177 - protein, 108 aa, Homo sapiens
QAVVTQPPSVSVSPGQTASITCSGDKLGDKYVSWYQQKPGQPPILVMYQDNRRPSGIPERFSGSNSGNTATLTISGTQAMDEADYYCQTWDSSTASYVFGTGTKVTVL

SEQ ID NO: 178 - protein, 108 aa, Homo sapiens
QSALTQPPSVSVAPGKTAMITCGGTNIRSQNVHWYQQKPGQAPVLVIYWDSSRPSGIPERISGSNSGSTATLTISRVEAGDEADYYCQVWDATRDHVVFGGGTKVTVL

SEQ ID NO: 179 - protein, 111 aa, Homo sapiens
NFMLTQPHSVSESPGKTVTISCTGSSGSIASKYVQWYQQRPGSAPTSVIYEDNQRPSGVPDRFSGSIDRSSNSASLTISGLKTEDEADYHCQSYDSANRVVFGGGTKVTVL

SEQ ID NO: 180 - protein, 110 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTTVTVL

SEQ ID NO: 181 - protein, 108 aa, Homo sapiens
QSALTQPPSVSVAPGQTATVTCEGDTIRTKTVHWYQQKPGQAPVLVVCPDCARPPVIPERFTTANSENTATLTIRRVEAGDEADYYCQMWDGGSSLVVFGGGTKVTVL

SEQ ID NO: 182 - protein, 107 aa, Homo sapiens
EIVLTQSPSSLSASVGDRVTITCRASQSISSYLNWYQQKPGKAPKLLIYAASSLQSGVPSRFSGSGSGTDFTLTISSLQPEDFATYYCQQSYSTLRTFGQGTRLEIK

SEQ ID NO: 183 - DNA, 360 bp, Homo sapiens
  source 1..360: mol_type = other DNA
caggtgcagctggtgcagtctggggctgaggtgaagaagcctggggcctcagtaaaagtttcctgcaaggcatctggatactccttcaccagctactttattcactgggtccgacaggcccctggacaagggcttgagtggatgggaataatcaaccctgctggtggtaacacaaactacgcacagaggttccagggcagagtcaccatgaccagggacacgtccacgagcacagtctacatggagctgagtagcctgagatctgacgacacggccgtttattactgtgcgagggacaaagactacggtgaaggcgcttttgatttgtggggccaagggacaatggtcagcgtctcctca

SEQ ID NO: 184 - DNA, 381 bp, Homo sapiens
  source 1..381: mol_type = other DNA
caggtggagctacaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccagagagctcggatattgtagtagtaccagctgcccgtggggtgcttttgatatctggggccaaggaaccctggtcagcgtctcctca

SEQ ID NO: 185 - DNA, 369 bp, Homo sapiens
  source 1..369: mol_type = other DNA
caggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagtaaactatgatagtagtggtctaaggggtgcttttgatatctggggccaagggacaatggtcatcgtctcctca

SEQ ID NO: 186 - DNA, 372 bp, Homo sapiens
  source 1..372: mol_type = other DNA
gaagtgcagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagagtccggcgtaagtggggactactactactacggtatggacgtctggggccaagggacaatggtcaccgtctcctca

SEQ ID NO: 187 - DNA, 369 bp, Homo sapiens
  source 1..369: mol_type = other DNA
caggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagtaaactatgatagtagtggtctaaggggtgcttttgatatctggggccaagggaccacggtcaccgtctcctca

SEQ ID NO: 188 - DNA, 363 bp, Homo sapiens
  source 1..363: mol_type = other DNA
caggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatcacgagcagtggctggtattggtttgactactggggccagggcaccctggtcaccgtctcctca

SEQ ID NO: 189 - DNA, 369 bp, Homo sapiens
  source 1..369: mol_type = other DNA
caggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagtaaactatgatagtagtggtctaaggggtgcttttgatatctggggccaagggacaatggtcaccgtctcctca

SEQ ID NO: 190 - DNA, 360 bp, Homo sapiens
  source 1..360: mol_type = other DNA
caggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatatagcactaccctactacggtatggacgtctggggccaagggacaatggtcaccgtctcttca

SEQ ID NO: 191 - DNA, 384 bp, Homo sapiens
  source 1..384: mol_type = other DNA
caggtgcagctggtgcagtctggggctgaggtgaagaagcctgggtcctcggtgaaggtctcctgcaagacttctggaggcaccttcagcagctatgctatcagctgggtgcgacaggcccctggacaagggcttgagtggatgggagggatcatccctatctctggtacagcaaactacgcacagaagttgcagggcagagtcacgattaccgcggacgaatccacgagcacagcctacatggagctgagcagcctgagatctgaggacacggccgtgtattactgtgggagaggtgattattgcaatggtggtagctgctcgtataccaactactacggtatggacgtctggggccaagggacaatggtcaccgtctcttca

SEQ ID NO: 192 - DNA, 372 bp, Homo sapiens
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagataaggcccatgatagtagtggttattatgatgcttttgatatctggggccaagggaccacggtcaccgtctcctca

SEQ ID NO: 193 - DNA, 384 bp, Homo sapiens
  source 1..384: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaactaccacgggccggacatcagtctcacgtattacgatttttggagtggttatcccgtctggggccagggaaccctggtcaccgtctcctca

SEQ ID NO: 194 - DNA, 384 bp, Homo sapiens
  source 1..384: mol_type = other DNA
gaagtgcagctggtgcagtctggggctgaggtgaagaagcctgggtcctcggtgaaggtctcctgcaaggcttctggaggcaccttcagcagctatgctatcacctgggtgcgacaggcccctggacaagggcttgagtggatgggagggatcatccctattgttggtacagcaaactacgcacagaagttccagggcagactcacgattaccgcggacgaatccacgagcacagcctacatggagctgagcagcctgagatctgaggacacggccgtgtattactgtgcgagaggggattattgtagtggtggtagctgctcgtataccaactactacggtatggacgtctggggccaagggaccacggtcaccgtctcttca

SEQ ID NO: 195 - DNA, 360 bp, Homo sapiens
  source 1..360: mol_type = other DNA
caggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatcggggtctactactacggtatggacgtctggggccaagggaccacggtcaccgtctcctca

SEQ ID NO: 196 - DNA, 369 bp, Homo sapiens
  source 1..369: mol_type = other DNA
caggtggagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagtaaactatgatagtagtggtctaaggggtgcttttgatatctggggccaagggaccacggtcaccgtctcttca

SEQ ID NO: 197 - DNA, 357 bp, Homo sapiens
  source 1..357: mol_type = other DNA
caggtggagctgcaggagtcgggcccaggactggtgcagccttcggggaccctgtccctcacctgcgctgtctctggtggctccatggggagtagtaactggtggagttgggtccgccagcccccagggaaggggctggagtggattggcgaagtctatcatactgggagcaccaactacaacccgtccctccggagtcgagtcaccatatcagtcgacaagtcgaaaaaacagttctccctggagttgacctctgtgaccgccgcggacacggccgtatatttctgtgcgagagatcttacggtgacaggttcttttgatatttggggccaagggacaatggtcaccgtctcctca

SEQ ID NO: 198 - DNA, 369 bp, Homo sapiens
  source 1..369: mol_type = other DNA
caggtgcagctggtgcaatctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagtgaagtcgtattactatgactctaatgatgcttttgatatctggggccaagggacaatggtcagcgtctcctca

SEQ ID NO: 199 - DNA, 360 bp, Homo sapiens
  source 1..360: mol_type = other DNA
caggtggagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatcggggtctactactacggtatggacgtctggggccaagggaccacggtcagcgtctcctca

SEQ ID NO: 200 - DNA, 363 bp, Homo sapiens
  source 1..363: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggcctgggggtcctggcgtactacggtatggacatctggggccaagggacaatggtcatcgtctcctca

SEQ ID NO: 201 - DNA, 378 bp, Homo sapiens
  source 1..378: mol_type = other DNA
gaagtgcagctggtgcagtctggggctgaggtgaagaagcctggggcctcagtgaaggtctcctgcaaggcttctggatacaccttcaccggctactatatgcactgggtgcgacaggcccctggacacgggcttgagtggatgggatggatcaaccctaacagtggtggcacaaactatgcacagaagtttcagggcagggtcaccatgaccagggacacgtccatcagcacagcctacatggaactgagcgggctgagatctgacgacacggccgtgtattactgtgcgagagataagggggcgtggagtgggagctacccagggtacaactggttcgacccctggggccagggaaccctggtcaccgtctcctca

SEQ ID NO: 202 - DNA, 354 bp, Homo sapiens
  source 1..354: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagggggactacctatgatgcttttgatatctggggccaagggaccacggtcagcgtctcctca

SEQ ID NO: 203 - DNA, 381 bp, Homo sapiens
  source 1..381: mol_type = other DNA
gaagtgcagctggtgcagtctgggcctgaggtgaggaagcctggggcctcagtccagatttcctgcaaggcatctggttacaccttcaccacctcctatatacactgggtgcgacaggcccctggacaaggccttgagtggttgggattagtcaaccctaatgatggcagtacaacctacacacagaatttccagggccgagtcaccatgaccagcgacacgtccacgagcacagtgtacatggaattgagcagggtgacatatgcggacacggccgtttattactgtgcgaaagatattcgagattatgtttgggggacttatcgttatgaaagtgcttttgatatctggggccaagggaccacggtcaccgtctcctca

SEQ ID NO: 204 - DNA, 384 bp, Homo sapiens
  source 1..384: mol_type = other DNA
gaggtgcagctggtgcagtctggggctgaggtgaagaagcctgggtcctcggtgaaggtctcctgcaagacttctggaggcaccttcagcagctatgctatcagctgggtgcgacaggcccctggacaagggcttgagtggatgggagggatcatccctatctctggtacagcaaactacccacagaagttgcagggcagggtcacgattaccgcggacgaatccacgagcacagcctacatggagctgagcagcctgagatctgaggacacggccatgtattactgtgcgagaggtgattattgtaatggtggtagctgctcgtataccaactactacggtatggacgtctggggccaagggaccacggtcatcgtctcctca

SEQ ID NO: 205 - DNA, 372 bp, Homo sapiens
  source 1..372: mol_type = other DNA
caggtgcagctggtgcagtctggagctgaggtgaagaagcctggggcctcagtgaaggtctcctgcaaggtttctggttacagttttagcagccacggtatcagctgggtgcgacaggcccctggacaagggcttgagtggatgggatggatcagcgcttacaatggtaacacaaactatgcacagaacctccagggcagagtccgcatgaccacagatacatccacgaacacagcccacatggaactgaggagcctgagatctgacgacacggccgtctactactgtgcgagagatcaggacgaagcaaactggaacaacaactactactttgactactggggccagggaaccctggtcaccgtctcctca

SEQ ID NO: 206 - DNA, 366 bp, Homo sapiens
  source 1..366: mol_type = other DNA
caggtgcagctggtgcaatctggggctgaggtgaagaagcctgggtcctcagtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagaggctacgccattcatgattcttggtgcttttgatatctggggccaagggacaatggtcatcgtctcctca

SEQ ID NO: 207 - DNA, 348 bp, Homo sapiens
  source 1..348: mol_type = other DNA
caggtgcagctggtgcagtctggagcagaggtgaaaaagcccggggagtctctgaagatctcctgtaagggttctggatacagcttttccacctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagagggccctacggtatggacgtctggggccaagggaccacggtcagcgtctcctca

SEQ ID NO: 208 - DNA, 363 bp, Homo sapiens
  source 1..363: mol_type = other DNA
caggtgcagctgttgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagtagttactatggttcggggacgacttggttcgacccctggggccagggaaccctggtcaccgtctcctca

SEQ ID NO: 209 - DNA, 333 bp, Homo sapiens
  source 1..333: mol_type = other DNA
cagtctgccctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttgggtattcggcggagggaccaaggtcaccgtccta

SEQ ID NO: 210 - DNA, 330 bp, Homo sapiens
  source 1..330: mol_type = other DNA
caggctgtggtgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgccgtggtattcggcggagggacccagctgaccgtccta

SEQ ID NO: 211 - DNA, 321 bp, Homo sapiens
  source 1..321: mol_type = other DNA
gatattgtgatgactcagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgatcaccttcggccaagggacacgactggagattaaa

SEQ ID NO: 212 - DNA, 321 bp, Homo sapiens
  source 1..321: mol_type = other DNA
gccatccagttgacccagtctccatcctccctgtctgcatctgtaggagacagagtcaccatcacttgccaggcgagtcaagacattaagaactatttaaattggtatcagcagagaccagggaaagcccctaaactcctgatctacgatgcatccaatttggaaaccggggtcccgtcaaggttcagtggaggtggatctgggacagattttactttcaccatcagcagcctgcagcctgaagatatcgcaacatattactgtcaacactatgatagtctcccgatcaccttcggccaagggacacgactggagattaaa

SEQ ID NO: 213 - DNA, 318 bp, Homo sapiens
  source 1..318: mol_type = other DNA
gccatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccccaccttcggccaagggacacgactggagattaaa

SEQ ID NO: 214 - DNA, 330 bp, Homo sapiens
  source 1..330: mol_type = other DNA
cagcctgtgctgactcagccaccctcagcgtctgggacccccgggcagagggtcaccatctcttgttctggaagcaactccaacatcggaagtaatgctgtaaactggtaccagcagctcccaggaacggcccccaaactcctcatccacagtcataatcagaggccctcaggggtccctgagcgattctctggctccaagtctggcacctcagcctccctggccatcagtggactccagtctgaggatgaggctgattattactgtgctgcatgggatgacagcctgaatggtccggttttcggcggagggaccaagctgaccgtccta

SEQ ID NO: 215 - DNA, 321 bp, Homo sapiens
  source 1..321: mol_type = other DNA
aacatccagatgacccagtctccatcgtccctgtctgcatctgtaggagacagagtcaccatcacttgccaggcgagtcagtacattagcaactctttaaattggtatcagcaaaaaccagggaaagcccctaacctcctcctctacgacgcaaccaatttggaaccaggggtcccatcaaggttcagtggaagtggatctcggacagatttcaccttaaccatcagcagcctgcagcctgaagatattgcaacatattactgtcaacaatatcataatctcccccccaccttcggccaagggacacgactggagatcaaa

SEQ ID NO: 216 - DNA, 330 bp, Homo sapiens
  source 1..330: mol_type = other DNA
cagtctgtgctgactcagccaccctcagcgtctgggacccccgggcagaaagtcaccatctcttgttctggaagcagctccaatatcggaagtagtcctgtaaactggtaccagcagctcccagggccggcccccaaactcctcatccatagtaataatcagcggccctcaggggtccctgaccgattctctggctccaagtctggcacctcagcctccctggccatcagtggactccagtctgacgatgaggctgattattactgtgcagcatgggatgacagcctgaatgcttatgtcttcggaactgggaccgagctgaccgtccta

SEQ ID NO: 217 - DNA, 330 bp, Homo sapiens
  source 1..330: mol_type = other DNA
caggctgtggtgactcagccaccctcagcgtctgggacccccgggcagaggtcaaccatctcttgttctggaggcagcgccaacatcggcagtaattctgtccactggtaccagcagttcccaggaacggcccccaaactcctcatctttaataataatcagcggccttcaggggtccctgaccgtttctctggctccaagtctggcacctcagcctccctggccatcagtgggctccagtctgaggatgaggctgattatttctgtgcagcatgggatgacagtctgaagggtccggtgttcggcggagggaccaaggtcaccgtccta

SEQ ID NO: 218 - DNA, 333 bp, Homo sapiens
  source 1..333: mol_type = other DNA
cagtctgccctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggcctttatgtcttcggaactgggaccaaggtcaccgtccta

SEQ ID NO: 219 - DNA, 330 bp, Homo sapiens
  source 1..330: mol_type = other DNA
cagtctgccctgactcagccgcactctgtgtcggagtctccggggaagacggtaaccatctcctgcacccgcagcagtggcagcattgccaacaatcatgtgcagtggtaccagcagcgcccgggcagtgcccccatcattgtgatctatgaggataaccaaagaccctctggggtccctgatcggttctctggctccatcgacagctcctccaactctgcctccctcaccctctctggactgaagactgaggacgaggctgcctactactgtcagtcttatgatgacaacaactgggtattcggcggagggaccaaggtcaccgtccta

SEQ ID NO: 220 - DNA, 330 bp, Homo sapiens
  source 1..330: mol_type = other DNA
caggctgtggtgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaaggtcaccgtccta

SEQ ID NO: 221 - DNA, 321 bp, Homo sapiens
  source 1..321: mol_type = other DNA
gacgtccagatgacccagtctccatcctccctgtctgcatctgttggagacagagtcaccatcacttgccgggcaggtcagagcgttggcacctatttaaattggtatcagcagaaaccagggaaagcccctaagctcctgatctatgctgcatccactttgcaaagtggggtcccatcaaggttcagtggcagtggatctgggacagatttcactctcaccatcagcagtctgcaacctgaagattttgcaacttactactgtcaacagagttacagtacccctatcaccttcggccaagggacacgactggagattaaa

SEQ ID NO: 222 - DNA, 318 bp, Homo sapiens
  source 1..318: mol_type = other DNA
gacgtccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccccaccttcggccaagggacacgactggagattaaa

SEQ ID NO: 223 - DNA, 330 bp, Homo sapiens
  source 1..330: mol_type = other DNA
caggctgtggtgactcagcctcgctcagtgtccgggtctcctggacagtcagtcaccatctcctgcactggaagcagcagtgatgttggtgcttataactatgtctcctggtaccaacaacacccagggaaagcccccaaattgataatttctgatgtcagtgattggccctcaggggtccctgatcgcttctctggctccaagtctggcaacacggcctccctgaccatctctgggctccaggctgacgatgaggctgattattactgcttctcatatgcaggcaactacaattgggtgttcggcggagggaccaagctgaccgtccta

SEQ ID NO: 224 - DNA, 336 bp, Homo sapiens
  source 1..336: mol_type = other DNA
gatgttgtgatgactcagtctccactctccctgcccgtcacccctggagagccggcctccatctcctgcaggtctagtcagagcctcctgcatagtaatggatacaactatttggactggtacatgcagaagccagggcagtctccacaggtcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcacacttttggccaggggactacagtggatatcaaa

SEQ ID NO: 225 - DNA, 330 bp, Homo sapiens
  source 1..330: mol_type = other DNA
cagtctgccctgactcagccaccctcagcgtctgggacccccgggcagaaagtcaccatctcttgttctggaagcagctccaatatcggaagtagtcctgtaaactggtaccagcagttcccagggccggcccccaagctcctcattcatagtaataatcagcggccctcaggggtccctgaccgattctctggctccaagtctggcacctcagcctccctggccatcagtggactccagtctgacgatgaggctgattattactgtgcagcatgggatgacagcctgaatgcttatgtcttcggaactgggaccaaggtcaccgtccta

SEQ ID NO: 226 - DNA, 321 bp, Homo sapiens
  source 1..321: mol_type = other DNA
gccatccagatgacccagtctccatcttctgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcactatcagcagcctgcagcctgaagattttgcaacttactattgtcaacaggctaacagtttccctctcactttcggcggagggaccaaagtggatatcaaa

SEQ ID NO: 227 - DNA, 330 bp, Homo sapiens
  source 1..330: mol_type = other DNA
caggctgtggtgactcagccaccctcagcgtctgggacccccgggcagagggtcaccatctcttgttctggaagcagctccaccatcggaactaatcctgtaaactggtaccggcacctcccaggaacggcccccaaactcctcatctatagtaataatcagcggccctcaggggtccctgaccgagtctctggctccaagtccggcacctcagcctccctggccatcagtgggctccagtctgaggatgaggctgattattactgtgcaacattggatgacagcctgaatggttatgtcttcggaactgggaccaaggtcaccgtccta

SEQ ID NO: 228 - DNA, 333 bp, Homo sapiens; identical to SEQ ID NO: 218
  source 1..333: mol_type = other DNA

SEQ ID NO: 229 - DNA, 324 bp, Homo sapiens
  source 1..324: mol_type = other DNA
caggctgtggtgactcagccaccctcagtgtccgtgtccccaggacagacagccagcatcacctgctctggagataaattgggagataaatatgtttcctggtatcaacagaagccaggccagccccctatactggtcatgtatcaagataacaggcggccctcagggatccctgagcgattctctggctccaactctggcaacacagccactctgaccatcagcgggacccaggctatggatgaggctgactattattgtcagacgtgggacagcagcactgcttcttatgtcttcggaactgggaccaaggtcaccgtccta

SEQ ID NO: 230 - DNA, 324 bp, Homo sapiens
  source 1..324: mol_type = other DNA
cagtctgccctgactcagccaccctcagtgtcagtggccccaggaaagacggccatgattacctgtgggggcaccaacattagaagtcaaaatgtgcattggtaccagcagaagccaggccaggcccctgtcctggtcatctattgggatagtagccggccctcagggatccctgagcgaatctctggctccaactctgggagcacggccaccctgaccatcagcagggtcgaagccggggatgaggccgactattactgtcaggtgtgggatgctactcgtgatcatgtggtattcggcggagggaccaaggtcaccgtccta

SEQ ID NO: 231 - DNA, 333 bp, Homo sapiens
  source 1..333: mol_type = other DNA
aattttatgctgactcagccccactctgtgtcggagtctccggggaagacggtaaccatctcctgcaccggcagcagtggcagcattgccagcaaatatgtgcagtggtaccagcagcgcccgggcagtgcccccaccagtgtgatctatgaggataaccaaagaccctctggggtccctgatcggttctctggctccatcgacagatcctccaactctgcctccctcaccatctctggactgaagactgaggacgaggctgactaccactgtcagtcttatgatagtgccaatcgagtggtgttcggcggagggaccaaggtcaccgtccta

SEQ ID NO: 232 - DNA, 330 bp, Homo sapiens
  source 1..330: mol_type = other DNA
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccacggtgaccgtcctg

SEQ ID NO: 233 - DNA, 324 bp, Homo sapiens
  source 1..324: mol_type = other DNA
cagtctgccctgactcagcccccctcggtgtcagtggccccaggacagacggccacagtgacctgtgagggagacaccattcgtactaaaaccgtccactggtaccagcagaagccaggccaggcccctgtgttggtcgtctgtcctgattgcgcccggcccccagtgatccctgagcgattcactaccgccaactctgaaaacacggccaccctgaccatcaggcgggtcgaggccggggatgaggccgactattactgtcaaatgtgggatggtggtagttctcttgttgtattcggcggagggaccaaggtcaccgtccta

SEQ ID NO: 234 - DNA, 321 bp, Homo sapiens
  source 1..321: mol_type = other DNA
gaaattgtgttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcaggacctttggccaagggacacgactggagattaaa

SEQ ID NO: 235 - RNA, 18 nt, unidentified
  source 1..18: mol_type = other RNA
gtaactgcattcaacttg

SEQ ID NO: 236 - RNA, 18 nt, unidentified
  source 1..18: mol_type = other RNA
ccagatcagtaggattga

SEQ ID NO: 237 - RNA, 18 nt, unidentified
  source 1..18: mol_type = other RNA
cctcatgcactgttgatt

SEQ ID NO: 238 - RNA, 18 nt, unidentified
  source 1..18: mol_type = other RNA
ccaccttccaaaatagcg

SEQ ID NO: 239 - RNA, 16 nt, unidentified
  source 1..16: mol_type = other RNA
caatgaacactaacca

SEQ ID NO: 240 - RNA, 19 nt, unidentified
  source 1..19: mol_type = other RNA
ccagtagtgtcgggagcaa

SEQ ID NO: 241 - RNA, 18 nt, unidentified
  source 1..18: mol_type = other RNA
gccacaatgccacagcta

SEQ ID NO: 242 - RNA, 19 nt, unidentified
  source 1..19: mol_type = other RNA
gagttggacccttgggcga

SEQ ID NO: 243 - RNA, 19 nt, unidentified
  source 1..19: mol_type = other RNA
ctgagttctggagccagta

SEQ ID NO: 244 - DNA, 58 bp, synthetic construct
  source 1..58: mol_type = other DNA
ccgggcattgattgtggacaacctactcgagtaggttgtccacaatcaatgctttttg

SEQ ID NO: 245 - DNA, 58 bp, synthetic construct
  source 1..58: mol_type = other DNA
ccggcgagttgactaagcctaactactcgagtagttaggcttagtcaactcgtttttg

SEQ ID NO: 246 - DNA, 20 bp, synthetic construct
  source 1..20: mol_type = other DNA
cgcagcaagacgggaaattg

SEQ ID NO: 247 - DNA, 20 bp, synthetic construct
  source 1..20: mol_type = other DNA
gggcgggagttactggaaaa

SEQ ID NO: 248 - DNA, 20 bp, synthetic construct
  source 1..20: mol_type = other DNA
acgtacaacctcacggggct

SEQ ID NO: 249 - DNA, 20 bp, synthetic construct
  source 1..20: mol_type = other DNA
tggccttcttccatccgcct

SEQ ID NO: 250 - DNA, 22 bp, synthetic construct
  source 1..22: mol_type = other DNA
tgtgagtgggatggtggaaggg

SEQ ID NO: 251 - DNA, 20 bp, synthetic construct
  source 1..20: mol_type = other DNA
agtgcatgaggtgggggtgt

SEQ ID NO: 252 - DNA, 20 bp, synthetic construct
  source 1..20: mol_type = other DNA
ggaggagttgcagcaaaaag

SEQ ID NO: 253 - DNA, 20 bp, synthetic construct
  source 1..20: mol_type = other DNA
tgtgtttgtgcccagaatgt

SEQ ID NO: 254 - DNA, 20 bp, synthetic construct
  source 1..20: mol_type = other DNA
ccatgcaggcacttactact

SEQ ID NO: 255 - DNA, 21 bp, synthetic construct
  source 1..21: mol_type = other DNA
ggcagtggtactgaagaagaa

SEQ ID NO: 256 - DNA, 22 bp, synthetic construct
  source 1..22: mol_type = other DNA
ccggattaatgtgactgaggtg

SEQ ID NO: 257 - DNA, 22 bp, synthetic construct
  source 1..22: mol_type = other DNA
gggtaacctggtactgactcta

SEQ ID NO: 258 - DNA, 22 bp, synthetic construct
  source 1..22: mol_type = other DNA
catcatcagcgtagtggctaaa

SEQ ID NO: 259 - DNA, 22 bp, synthetic construct
  source 1..22: mol_type = other DNA
gtaatgccaggtgaggagaatc

SEQ ID NO: 260 - DNA, 19 bp, synthetic construct
  source 1..19: mol_type = other DNA
cagcacacaccatcacaga

SEQ ID NO: 261 - DNA, 21 bp, synthetic construct
  source 1..21: mol_type = other DNA
gtcactccatgtcccaatctc

SEQ ID NO: 262 - DNA, 20 bp, synthetic construct
  source 1..20: mol_type = other DNA
ctgtgggtgacagcatctac

SEQ ID NO: 263 - DNA, 22 bp, synthetic construct
  source 1..22: mol_type = other DNA
ctttccacctcagggtgttatc

SEQ ID NO: 264 - DNA, 23 bp, synthetic construct
  source 1..23: mol_type = other DNA
gtcattccaaatatgagatgcgt

SEQ ID NO: 265 - DNA, 20 bp, synthetic construct
  source 1..20: mol_type = other DNA
gctatcacctcccctgtgtg

(19) United States
(12) Patent Application Publication
(10) Pub. No.: US 2025/0197513 A1
(43) Pub. Date: Jun. 19, 2025

(54) OSMR-SPECIFIC MONOCLONAL ANTIBODIES AND METHODS OF THEIR USE

(71) Applicants: THE BOARD OF REGENTS OF THE UNIVERSITY OF TEXAS SYSTEM, Austin, TX (US); THE MEDICAL COLLEGE OF WISCONSIN, INC., Milwaukee, WI (US)

(72) Inventors: Zhiqiang KU, Houston, TX (US); Ningyan ZHANG, Houston, TX (US); Zhiqiang AN, Houston, TX (US); Anjali GEETHADEVI, Milwaukee, WI (US); Sunila PRADEEP, Milwaukee, WI (US); Pradeep CHALUVALLY-RAGHAVAN, Milwaukee, WI (US)

(73) Assignees: THE BOARD OF REGENTS OF THE UNIVERSITY OF TEXAS SYSTEM, Austin, TX (US); THE MEDICAL COLLEGE OF WISCONSIN, INC., Milwaukee, WI (US)

(21) Appl. No.: 18/293,455

(22) PCT Filed: Jul. 28, 2022
(86) PCT No.: PCT/US2022/074276

§ 371 Date: Jan. 30, 2024

Related U.S. Application Data
(60) Provisional application No. 63/228,005, filed on Jul. 30, 2021.

(51) Int. Cl. C07K 16/28; A61K 39/00; A61K 40/31; A61K 45/06; A61K 47/68; A61P 35/00

(52) U.S. Cl. CPC C07K 16/2866; A61K 40/31; A61K 45/06; A61K 47/68031; A61K 47/6849; A61P 35/00; A61K 2039/505; C07K 2317/24; C07K 2317/565

(57) ABSTRACT

Isolated or recombinant anti-OSMR monoclonal antibodies are provided. In some embodiments, the antibodies herein can be used for the detection, diagnosis and/or therapeutic treatment of human diseases, such as cancer.

### CROSS-REFERENCE TO RELATED APPLICATIONS

This application claims the benefit of U.S. Provisional Appl. No. 63/228,005, filed Jul. 30, 2021. The content of the foregoing application is relied upon and is incorporated by reference herein in its entirety.

### STATEMENT REGARDING FEDERALLY SPONSORED RESEARCH OR DEVELOPMENT

This invention was made with U.S. Government support under Grant No. R01CA229907 awarded by the National Institutes of Health. The government has certain rights in the invention.

### REFERENCE TO A SEQUENCE LISTING SUBMITTED VIA ELECTRONIC FILING

The content of the XML file of the sequence listing named “UTH-004PCT0_sequence_listing_ST26_FILED.XML” which is 253 KB in size was created on Jul. 26, 2022, and electronically submitted to the USPTO's Patent Center herewith the present application is incorporated by reference in its entirety.

### TECHNICAL FIELD

The present invention relates generally to the field of cancer biology. More particularly, it concerns Oncostatin M receptor (hereinafter, “OSMR”) targeting monoclonal antibodies for the treatment and detection of cancer.

### BACKGROUND

Ovarian cancer (“OC”) is among the most lethal gynecological malignancies and the fifth leading cause of cancer-related mortality in women in the United States. While patients with advanced ovarian cancer may respond initially to surgery, chemotherapy, and targeted therapy, many patients often see their cancers re-emerge, with nearly half of these patients not surviving beyond five years. A recent study using single-cell RNA sequencing (scRNA-seq) of cells collected from the ascites samples of high-grade serous ovarian cancer (or “HGSOC”) provided evidence for JAK/STAT3 signaling being a vulnerable and potential target for therapy in ovarian cancer cells and cancer associated fibroblast (Izar et al., 2020). This study suggested that cells in the ascites fluid microenvironment, such as cancer-associated fibroblasts (or “CAFs”), and tumor-associated macrophages (or “TAMs”), expressed high transcript levels of genes encoding secreted ligands that activate JAK/STAT3 pathway in cancer cells. The JAK/STAT3 pathway has been shown to be an important signaling mechanism required for the growth and progression of ovarian cancer (Chaluvally-Raghavan et al., 2016; Chen et al., 2020; Parashar et al., 2019; Rose-John, 2018; Yoshikawa et al., 2018). However direct targeting of STAT3 with small molecule inhibitors such as JSI-124 showed suboptimal potency, unfavorable pharmacokinetics (PK) properties, and their non-specific effects in non-cancerous cells and immune cells (Izar et al., 2020). These adverse effects are also partly due to the high sequence similarity and homology between STAT transcription factors as well as the issues associated with poor bioavailability of STAT inhibitors (Zou et al., 2020).

It is known that the signaling outcome such as cell division and migration through IL6-family ligands is via the activation of Janus kinases (Jaks) and transcription factors of the STAT family (Taga and Kishimoto, 1997). Upon stimulation by IL-6 subfamily of ligands such as IL6 (or “interleukin-6”), IL11, ciliary neurotrophic factor (or “CNTF”), leukemia inhibitory factor (or “LIF”), oncostatin M (hereinafter, “OSM”), cardiotrophin 1 (or “CT-1”), cardiotrophin-like cytokine (or “CLC”), IL27 and IL31, the cytoplasmic tail receptor-associated kinases like JAK1, and JAK2 are phosphorylated and activated, which then serve as the docking sites for STAT transcription factors with matching SH2 domains primarily STAT3 and STAT1 proteins (Murakami et al., 2019; Rose-John, 2018). As a consequence, STAT proteins become phosphorylated and dimerize, then translocate to the nucleus and upregulate genes which are important for cancer progression and metastasis (Murakami et al., 2019; Rose-John, 2018). IL-6 family cytokines and their receptors constitute interleukin-6 receptor (or “IL6R”), interleukin-11 receptor (or “IL11RA”), ciliary neurotrophic factor receptor (or “CNTFR”), leukemia inhibitory factor receptor (or “LIFR”), oncostatin M receptor (OSMR), interleukin-27 receptor (or “IL-27RA”), and interleukin-31 receptor (or “IL31RA”).

Signaling through OSMR is triggered by the binding of OSM to OSMR, which leads to heterodimerization of OSMR with interleukin-6 signal transducer (IL6ST; also known as glycoprotein 130 (or GP130). OSM also binds to leukemia inhibitor factor receptor (LIFR) and causes its heterodimerization with IL6ST. In addition, OSMR dimerizes with Interleukin-31 Receptor (IL31RA), when IL-31 binds to IL-31 (Tanaka and Miyajima, 2003). OSMR is identified as a key regulator for activating oncogenic pathways through JAK/STAT, MAPK, PKC isoforms and PI3K/AKT pathways in cancer cells (Demyanets et al., 2011; Kurosawa et al., 2015). However, OSMR as a potential therapeutic target for ovarian and other cancers has not been explored. There is a need for better diagnostics and treatments for ovarian cancer.

### SUMMARY

Monoclonal antibody compositions are now provided herein which may be used in diagnosis and/or treatment of cancers including, for example, certain types of ovarian cancer. Advantageously, this discovery provides its user with means to treat certain types of cancer (for example, ovarian cancer) that have failed to respond adequately to other conventional treatments.

Described herein are monoclonal antibodies that bind to OSMR. In further aspects, the provided OSMR-binding antibodies reduce cellular signaling mediated through at least the IL6/JAK/STAT3 signaling pathway and can be used to inhibit cancer cell proliferation.

Thus, in one aspect, there is provided an isolated or recombinant monoclonal antibody that specifically binds to OSMR. In certain aspects, an antibody that competes for the binding of OSMR with the B01, B02, B03, B04, B05, B06, B07, B08, B09, B010, B12, B13, B14, B16, B17, B18, B19, B21, H09, H10, H11, H12, H13, H14, H15, or H16 monoclonal antibody is provided. In certain aspects, the antibody may comprise all or part of the heavy chain variable region and/or light chain variable region of the B01, B02, B03, B04, B05, B06, B07, B08, B09, B010, B12, B13, B14, B16, B17, B18, B19, B21, H09, H10, H11, H12, H13, H14, H15, or H16 monoclonal antibodies.

In a further aspect, the antibody may comprise an amino acid sequence that corresponds to a first, second, and/or third complementarity determining region (CDR) from the light variable and/or heavy variable chain of the B01, B02, B03, B04, B05, B06, B07, B08, B09, B010, B12, B13, B14, B16, B17, B18, B19, B21, H09, H10, H11, H12, H13, H14, H15, or H16 monoclonal antibodies of the present embodiments.

In certain embodiments, the isolated antibody comprises CDR sequences at least 80%, 85%, 90%, 91%, 92%, 93%, 94%, 95%, 96%, 97%, 98%, 99% or 100% identical to the CDR regions of the B01, B02, B03, B04, B05, B06, B07, B08, B09, B010, B12, B13, B14, B16, B17, B18, B19, B21, H09, H10, H11, H12, H13, H14, H15, or H16 heavy and light chain amino acid sequences. In further aspects, an antibody comprises CDR regions identical to the B01, B02, B03, B04, B05, B06, B07, B08, B09, B010, B12, B13, B14, B16, B17, B18, B19, B21, H09, H10, H11, H12, H13, H14, H15, or H16 CDR regions, except for one or two amino acid substitutions, deletions, or insertions at one or more of the CDRs.

Thus, in some specific embodiments, an antibody of the present disclosure comprises (a) a first V_{H} CDR at least 80%, 85%, 90%, 91%, 92%, 93%, 94%, 95%, 96%, 97%, 98%, 99% or 100% identical to V_{H} CDR1 of B01 (SEQ ID NO: 1), B02 (SEQ ID NO: 4), B03 (SEQ ID NO: 7), B04 (SEQ ID NO: 10), B05 (SEQ ID NO: 13), B06 (SEQ ID NO: 16), B07 (SEQ ID NO: 19), B08 (SEQ ID NO: 22), B09 (SEQ ID NO: 25), B10 (SEQ ID NO: 28), B12 (SEQ ID NO: 31), B13 (SEQ ID NO: 34), B14 (SEQ ID NO: 37), B16 (SEQ ID NO: 40), B17 (SEQ ID NO: 43), B18 (SEQ ID NO: 46), B19 (SEQ ID NO: 49), B21 (SEQ ID NO: 52), H09 (SEQ ID NO: 55), H10 (SEQ ID NO: 58), H11 (SEQ ID NO: 61), H12 (SEQ ID NO: 64), H13 (SEQ ID NO: 67), H14 (SEQ ID NO: 70), H15 (SEQ ID NO: 73), or H16 (SEQ ID NO: 76); (b) a second V_{H} CDR at least 80%, 85%, 90%, 91%, 92%, 93%, 94%, 95%, 96%, 97%, 98%, 99% or 100% identical to V_{H} CDR2 of B01 (SEQ ID NO: 2), B02 (SEQ ID NO: 5), B03 (SEQ ID NO: 8), B04 (SEQ ID NO: 11), B05 (SEQ ID NO: 14), B06 (SEQ ID NO: 17), B07 (SEQ ID NO: 20), B08 (SEQ ID NO: 23), B09 (SEQ ID NO: 26), B10 (SEQ ID NO: 29), B12 (SEQ ID NO: 32), B13 (SEQ ID NO: 35), B14 (SEQ ID NO: 38), B16 (SEQ ID NO: 41), B17 (SEQ ID NO: 44), B18 (SEQ ID NO: 47), B19 (SEQ ID NO: 50), B21 (SEQ ID NO: 53), H09 (SEQ ID NO: 56), H10 (SEQ ID NO: 59), H11 (SEQ ID NO: 62), H12 (SEQ ID NO: 65), H13 (SEQ ID NO: 68), H14 (SEQ ID NO: 71), H15 (SEQ ID NO: 74), or H16 (SEQ ID NO: 77); (c) a third V_{H} CDR at least 80%, 85%, 90%, 91%, 92%, 93%, 94%, 95%, 96%, 97%, 98%, 99% or 100% identical to V_{H} CDR3 of B01 (SEQ ID NO: 3), B02 (SEQ ID NO: 6), B03 (SEQ ID NO: 9), B04 (SEQ ID NO: 12), B05 (SEQ ID NO: 15), B06 (SEQ ID NO: 18), B07 (SEQ ID NO: 21), B08 (SEQ ID NO: 24), B09 (SEQ ID NO: 27), B10 (SEQ ID NO: 30), B12 (SEQ ID NO: 33), B13 (SEQ ID NO: 36), B14 (SEQ ID NO: 39), B16 (SEQ ID NO: 42), B17 (SEQ ID NO: 45), B18 (SEQ ID NO: 48), B19 (SEQ ID NO: 51), B21 (SEQ ID NO: 54), H09 (SEQ ID NO: 57), H10 (SEQ ID NO: 60), H11 (SEQ ID NO: 63), H12 (SEQ ID NO: 68), H13 (SEQ ID NO: 69), H14 (SEQ ID NO: 72), H15 (SEQ ID NO: 76), or H16 (SEQ ID NO: 78); (d) a first V_{L} CDR at least 80%, 85%, 90%, 91%, 92%, 93%, 94%, 95%, 96%, 97%, 98%, 99% or 100% identical to V_{L} CDR1 of B01 (SEQ ID NO: 79), B02 (SEQ ID NO: 81), B03 (SEQ ID NO: 83), B04 (SEQ ID NO: 85), B05 (SEQ ID NO: 87), B06 (SEQ ID NO: 89), B07 (SEQ ID NO: 91), B08 (SEQ ID NO: 93), B09 (SEQ ID NO: 95), B10 (SEQ ID NO: 97), B12 (SEQ ID NO: 99), B13 (SEQ ID NO: 101), B14 (SEQ ID NO: 103), B16 (SEQ ID NO: 105), B17 (SEQ ID NO: 107), B18 (SEQ ID NO: 109), B19 (SEQ ID NO: 111), B21 (SEQ ID NO: 113), H09 (SEQ ID NO: 115), H10 (SEQ ID NO: 117), H11 (SEQ ID NO: 119), H12 (SEQ ID NO: 121), H13 (SEQ ID NO: 123), H14 (SEQ ID NO: 125), H15 (SEQ ID NO: 127), or H16 (SEQ ID NO: 129); (e) a second V_{L} CDR at least 80%, 85%, 90%, 91%, 92%, 93%, 94%, 95%, 96%, 97%, 98%, 99% or 100% identical to V_{L} CDR2 of a tripeptide selected from a group consisting of GNS, DNN, DAS, SHN, DAT, SNN, NNN, RNN, EDN, AAS, DVS, LGS, QDN, WDS, and PDC; and (f) a third V_{L} CDR at least 80%, 85%, 90%, 91%, 92%, 93%, 94%, 95%, 96%, 97%, 98%, 99% or 100% identical to V_{L} CDR3 of B01 (SEQ ID NO: 80), B02 (SEQ ID NO: 82), B03 (SEQ ID NO: 84), B04 (SEQ ID NO: 86), B05 (SEQ ID NO: 88), B06 (SEQ ID NO: 90), B07 (SEQ ID NO: 92), B08 (SEQ ID NO: 94), B09 (SEQ ID NO: 96), B10 (SEQ ID NO: 98), B12 (SEQ ID NO: 100), B13 (SEQ ID NO: 102), B14 (SEQ ID NO: 104), B16 (SEQ ID NO: 106), B17 (SEQ ID NO: 108), B18 (SEQ ID NO: 110), B19 (SEQ ID NO: 112), B21 (SEQ ID NO: 114), H09 (SEQ ID NO: 116), H10 (SEQ ID NO: 118), H11 (SEQ ID NO: 120), H12 (SEQ ID NO: 122), H13 (SEQ ID NO: 124), H14 (SEQ ID NO: 126), H15 (SEQ ID NO: 128), or H16 (SEQ ID NO: 130). In certain aspects, such an antibody is a humanized or de-immunized antibody comprising the foregoing CDRs on a human IgGs (e.g., IgG1, IgG2, IgG4, or a genetically modified IgG) backbone.

In yet another aspect, the present disclosure provides a recombinant polypeptide comprising an antibody V_{H} domain comprising CDRs 1-3 of the V_{H} domain of B01 (SEQ ID NOs: 1, 2, and 3); CDRs 1-3 of the V_{H} domain of B02 (SEQ ID NOs: 4, 5, and 6); CDRs 1-3 of the V_{H} domain of B03 (SEQ ID NOs: 7, 8, and 9); CDRs 1-3 of the V_{H} domain of B04 (SEQ ID NOs: 10, 11, and 12); CDRs 1-3 of the V_{H} domain of B05 (SEQ ID NOs: 13, 14, and 15); CDRs 1-3 of the V_{H} domain of B06 (SEQ ID NOs: 16, 17, and 18); CDRs 1-3 of the V_{H} domain of B07 (SEQ ID NOs: 19, 20, and 21); CDRs 1-3 of the V_{H} domain of B08 (SEQ ID NOs: 22, 23, and 24); CDRs 1-3 of the V_{H} domain of B09 (SEQ ID NOs: 25, 26, and 27); CDRs 1-3 of the V_{H} domain of B10 (SEQ ID NOs: 28, 29, and 30); CDRs 1-3 of the V_{H} domain of B12 (SEQ ID NOs: 31, 32, and 33); CDRs 1-3 of the V_{H} domain of B13 (SEQ ID NOs: 34, 35, and 36); CDRs 1-3 of the V_{H} domain of B14 (SEQ ID NOs: 37, 38, and 39); CDRs 1-3 of the V_{H} domain of B16 (SEQ ID NOs: 40, 41, and 42); CDRs 1-3 of the V_{H} domain of B17 (SEQ ID NOs: 43, 44, and 45); CDRs 1-3 of the V_{H} domain of B18 (SEQ ID NOs: 46, 47, and 48); CDRs 1-3 of the V_{H} domain of B19 (SEQ ID NOs: 49, 50, and 51); CDRs 1-3 of the V_{H} domain of B21 (SEQ ID NOs: 52, 53, and 54); CDRs 1-3 of the Vu domain of H09 (SEQ ID NOs: 55, 56, and 57); CDRs 1-3 of the V_{H} domain of H10 (SEQ ID NOs: 58, 59, 60); CDRs 1-3 of the V_{H} domain of H11 (SEQ ID NOs: 61, 62, and 63); CDRs 1-3 of the V_{H} domain of H12 (SEQ ID NOs: 64, 65, and 66); CDRs 1-3 of the V_{H} domain of H13 (SEQ ID NOs: 67, 68, and 69); CDRs 1-3 of the V_{H} domain of H14 (SEQ ID NOs: 70, 71, and 72); CDRs 1-3 of the V_{H} domain of B15 (SEQ ID NOs: 73, 74, and 75); or CDRs 1-3 of the V_{H} domain of B16 (SEQ ID NOs: 76, 77, and 78).

In yet another aspect, the present disclosure provides a recombinant polypeptide comprising an antibody V_{L} domain comprising CDRs 1-3 of the V_{L} domain of B01 (SEQ ID NO: 79, the tripeptide GNS, and SEQ ID NO: 80); CDRs 1-3 of the V_{L} domain of B02 (SEQ ID NO: 81, the tripeptide DNN, and SEQ ID NO: 82); CDRs 1-3 of the V_{L} domain of B03 (SEQ ID NO: 83, the tripeptide DAS, and SEQ ID NO: 84); CDRs 1-3 of the V_{L} domain of B04 (SEQ ID NO: 85, the tripeptide DAS, and SEQ ID NO: 86); CDRs 1-3 of the V_{L} domain of B05 (SEQ ID NO: 87, the tripeptide DAS, and SEQ ID NO: 88); CDRs 1-3 of the V_{L} domain of B06 (SEQ ID NO: 89, the tripeptide SHN, and SEQ ID NO: 90); CDRs 1-3 of the V_{L} domain of B07 (SEQ ID NO: 91, the tripeptide DAT, and SEQ ID NO: 92); CDRs 1-3 of the V_{L} domain of B08 (SEQ ID NO: 93, the tripeptide SNN, and SEQ ID NO: 94); CDRs 1-3 of the V_{L} domain of B09 (SEQ ID NO: 95, the tripeptide NNN, and SEQ ID NO: 96); CDRs 1-3 of the V_{L} domain of B10 (SEQ ID NO: 97, the tripeptide RNN, and SEQ ID NO: 98); CDRs 1-3 of the V_{L} domain of B12 (SEQ ID NO: 99, the tripeptide EDN, and SEQ ID NO: 100); CDRs 1-3 of the V_{L} domain of B13 (SEQ ID NO: 101, the tripeptide SNN, and SEQ ID NO: 102); CDRs 1-3 of the V_{L} domain of B14 (SEQ ID NO: 103, the tripeptide AAS, and SEQ ID NO: 104); CDRs 1-3 of the V_{L} domain of B16 (SEQ ID NO: 105, the tripeptide DAS, and SEQ ID NO: 106); CDRs 1-3 of the V_{L} domain of B17 (SEQ ID NO: 107, the tripeptide DVS, and SEQ ID NO: 108); CDRs 1-3 of the V_{L} domain of B18 (SEQ ID NO: 109, the tripeptide LGS, and SEQ ID NO: 110); CDRs 1-3 of the V_{L} domain of B19 (SEQ ID NO: 111, the tripeptide SNN, and SEQ ID NO: 112); CDRs 1-3 of the V_{L} domain of B21 (SEQ ID NO: 113, the tripeptide AAS, and SEQ ID NO: 114); CDRs 1-3 of the V_{L} domain of H09 (SEQ ID NO: 115, the tripeptide SNN, and SEQ ID NO: 116); CDRs 1-3 of the V_{L} domain of H10 (SEQ ID NO: 117, the tripeptide SNN, and SEQ ID NO: 118); CDRs 1-3 of the V_{L} domain of H11 (SEQ ID NO: 119, the tripeptide QDN, and SEQ ID NO: 120); CDRs 1-3 of the V_{L} domain of H12 (SEQ ID NO: 121, the tripeptide WDS, and SEQ ID NO: 122); CDRs 1-3 of the V_{L} domain of H13 (SEQ ID NO: 123, the tripeptide EDN, and SEQ ID NO: 124); CDRs 1-3 of the V_{L} domain of H14 (SEQ ID NO: 125, the tripeptide SNN, and SEQ ID NO: 126); CDRs 1-3 of the V_{L} domain of H15 (SEQ ID NO: 127, the tripeptide PDC, and SEQ ID NO: 128); or CDRs 1-3 of the V_{L} domain of H16 (SEQ ID NO: 129, the tripeptide AAS, and SEQ ID NO: 130).

In yet another aspect, the present disclosure provides a composition comprising the antibody or recombinant polypeptide of any one of the above embodiments.

In yet another aspect, the present disclosure provides a host cell comprising one or more polynucleotide molecule(s) encoding an antibody or a recombinant polypeptide, or portion thereof, of any one of the above embodiments (see, for example, the polynucleotides of SEQ ID NOS. 183-234).

In yet another aspect, the present disclosure provides a method for treating a subject having a cancer comprising administering an effective amount of an antibody of any one of the above embodiments to the subject.

The numbers, E5, E6, and E7 and the like are used interchangeably herein with 105, 106, and 107, respectively, and the like.

The term “comprising” and variations thereof (e.g., comprises, includes, etc.) do not have a limiting meaning where these terms appear in the description and claims.

As used herein, “a”, “an”, “the,” “at least one,” and “one or more” are used interchangeably, unless the context clearly dictates otherwise.

Also herein, the recitations of numerical ranges by endpoints include all numbers subsumed within that range (e.g., 500 to 7000 nm includes 500, 530, 551, 575, 583, 592, 600, 620, 650, 700, etc.).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

Unless otherwise indicated, all numbers in the description and the claims expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

The above summary of the present invention is not intended to describe each disclosed embodiment or every implementation of the present invention. The description that follows more particularly exemplifies illustrative embodiments.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1A-F. Oncostatin M (OSM) family genes are associated with chemoresistance in Ovarian Cancer through OSM Receptor dimerization. A Heatmap clustergram of differentially expressed genes greater or lesser than 1.5-fold change generated from IL-6 signaling qPCR array in A2780-CP cisplatin resistant cells vs. A2780 sensitive cells in triplicates. Genes that are endogenously upregulated or downregulated are represented by red or green color respectively. C Western blot analysis of the top candidate genes obtained from (a) were performed in cisplatin resistant ovarian cancer cell lines A2780, PEO4 and patient derived ovarian carcinoma cell line SL-3 and in the parent version of A2780, PEO1 and SL-3. D Endogenous levels of human OSM, LIF, IL31, IL6 and IL8 in culture supernatant of A2780-CP vs. A2780 sensitive cells and SL-3-CP vs. SL-3 sensitive cell lines were determined by Luminex multiplex ELISA. E, OSMR was immunoprecipitated (IP) from A2780-CP and A2780 sensitive cells that were treated with OSM (100 ng/mL) and crosslinked with BS3 agent. Monomers and dimers obtained by IP were resolved on SDS-PAGE and immunoblotted using OSMR for its monomers and dimer first; then the membranes were stripped and immunoblotted with IL6ST for heterodimers. F. Western blot analysis of A2780 sensitive and A2780-Cis and OVCAR8 and OVCAR8-Cis cisplatin resistant cells showing the endogenous expression of OSMR and pSTAT3. ****P<0.0001, ***P<0.001. Student's t test (two tailed, unpaired) (Dunnett's multiple comparison) was performed to determine significance between different groups. Data represent means±SEM.

FIG. 2A-F. Anti-OSMR antibody increases the chemosensitivity of Cisplatin resistant ovarian cancer cells in vitro by reducing Cisplatin IC50 and spheroid formation by inhibiting OSMR and STAT3 phosphorylation. (A) Cell viability of A2780 sensitive and A2780-CP cisplatin resistant cells (B) OVCAR8 sensitive and OVCAR8-Cis resistant cells (C) SL3 and SL3 Cis Resistant cells after treatment with different concentrations of Cisplatin for 48 h were determined. The IC50 of cisplatin of three cell lines are shown in the respective boxes. (D-F) Cell viability of A2780 sensitive and A2780-CP cisplatin resistant cells OVCAR8 sensitive and OVCAR8-Cis resistant cells, SL3 and SL3 Cis showing reduction in IC50 after treatment with B21 anti-OSMR antibody (10 μg/mL) in combination with different concentrations of Cisplatin for 48 h. d 3D spheroid formation assay in A2780-CP after treatment with OSM and B14 and B21 anti-OSMR antibodies (10 μg/mL each) in the presence of OSM (100 ng/ml).

FIG. 3A-D Anti-OSMR antibody increases the chemosensitivity of Cisplatin resistant ovarian cancer cells in vitro by reducing spheroid forming ability. (A-B) 3D spheroid formation assay of luciferase reporter genes stably expressing A2780-Cis, and SL3-Cis cells were treated with B14 and B21 anti-OSMR antibody (10 μg/mL each) followed by exogenous induction of recombinant human OSM (100 ng/mL) was performed for the indicated days and photographed. (C-D) 3D spheroid formation assay in A2780-Cis, and SL3-Cis after treatment with B14 and B21 anti-OSMR antibody (10 μg/mL each) alone and in combination with Cisplatin (10 μM). Bar graph showing luminescence intensity corresponds to number of spheroids performed using 3D-viability assay. Size of spheroids shown are view per 4 fields per treatment. Scale bar, 500 μM. Student's t test (two tailed, unpaired) was performed. Data represent means±SEM. **P≤0.01, *P≤0.05.

FIG. 4A-B. Anti-OSMR antibodies decrease the colony forming ability and phosphorylation of STAT3 in ovarian cancer cells and cisplatin resistant cells. (A) OVCAR8 and OVCAR8-Cis resistant cells were treated with recombinant human OSM (100 ng/mL), and B14 and B21 antibody (10 μg/mL) and then seeded for 10 days to assess the colony forming ability. Control IgG is the isotype control. (B) OVCAR8 and OVCAR8-Cis resistant cells were treated with B14 and B21 antibody (10 μg/mL) followed by recombinant human OSM (100 ng/mL), and Western blot analysis for OSMR and its downstream targets were performed.

FIG. 5. A-H. Anti-OSMR antibody increases the chemosensitivity of ovarian cancer cells towards Cisplatin in vivo. A-B Representative image of tumor growth was determined by bioluminescence IVIS100 imager from A2780-Luc+ and A2780-Cis-Luc+ tumor bearing mice treated with Isotype control IgG without and with OSM stimulation, OSM+ B14 and OSM+B21 anti-OSMR antibodies. Images were taken at the indicated days. C-D Quantitative assessment of luciferase signal intensity, and E-F total weight of the tumor in treatment groups from (a) were determined. G Representative western blots showing the protein expression of indicated proteins from tumor tissues of mice from ‘A’. H Proposed model showing the efficacy and mechanism of B14 and B21 anti-OSMR mAb in inhibiting the tumor progression and promoting cell survival and modulating sensitivity of tumor cells to Cisplatin. Dunnett's multiple comparison test and Student's t test (two tailed, unpaired) were performed in (C-E). Data represent means±SEM in (C-E). ****P<0.0001, ***P<0.001, **P≤0.01, *P≤0.05.

FIG. 6A-H. Knockdown of OSMR significantly inhibit the proliferation and migration of ovarian cancer cells and fibroblasts. A-D, CCK8 assay demonstrating proliferation in ovarian cancer cells (HEYA8 and OVCAR4), fibroblasts, macrophages (THP1), and Endothelial cells (RF24). Cells were transfected with indicated siRNA of IL6 family receptors for 24 h and CCK8 proliferation assay was performed at the indicated time points. E-H Trans-well migration assay in ovarian cancer cells (HEYA8 and OVCAR4), Macrophages (THP1), Endothelial cells (RF24) and Fibroblasts cells. Cells were transfected with indicated siRNA of IL6 family receptors for 24 h and migration assay was performed for 12 h. Student's t test (two tailed, unpaired) was performed in (A-H) w.r.t control siRNA. Data represent means±SEM. ****P£ 0.0001, ***P£0.001, **P£0.01, *P£ 0.05, #P£0.001, $P£ 0.0001, ns: not significant.

FIG. 7A-C. OSMR expression is associated with malignant characteristics in ovarian cancer. A. GSEA analysis demonstrating the enrichment score of indicated functional annotation marks based on OSM expression in the TCGA ovarian cancer samples. ES: enrichment score, NES: normalized enrichment score. B. Representative images from ovarian cancer tissue microarray core showing OSM expression immunostained for OSM and scanned using Aperio Scan Scope (Aperio Technologies). Normal (N), Normal adjacent tumor (NAT), Malignant stage I, II and III are marked. Scale bar, 100 μm. C. Histogram shows the relative size of spheroids as in at the indicated time points. Student's t test and Dunnett's multiple comparison test were performed in (C). Data represent means±SEM. ****P£ 0.0001, and **P£0.01

FIG. 8A-J. High OSMR expression promotes oncogenic signaling in ovarian cancer. A, OVCAR4 cells were stimulated with OSM for 24 h and cell lysates were prepared, and the levels of phospho-kinase proteins were quantitated using Phospho-protein kinase array membrane. B, the histograms represent mean values of densitometry readings of the significantly altered Phospho-kinase proteins on the array membrane marked in white squares. C, HEYA8 and OVCAR5 cells were stably transfected with pUNO1-OSMR overexpression plasmid. C#1 to C#3 refers to different clones selected after stable transfection and western blot was performed for the indicated proteins. D, HEYA8 and OVCAR5 were stably transfected with clone #1 and clone #3 respectively and western blot was performed for the indicated proteins. E, HEYA8 and OVCAR5 cells were stably transfected with pUNO-1-OSMR overexpression plasmid and 1200 cells were seeded on 6 well plate for colony forming assay. Colonies were stained with 0.5% crystal violet and photographed on Day 10. F, Crystal violet-stained colonies were eluted in 10% acetic acid and quantitated. G, HEYA8 cells stably overexpressed with pUNO1-Control vector and clone #1 of pUNO1-OSMR were seeded for 12 h and 16 h for migration and invasion respectively. Scale bar, 100 μm. H, Crystal violet-stained migrated and invaded cells were eluted in 10% acetic acid and quantitated. I, Wound healing assay in HEYA8 stably overexpressed with pUNO1-Control vector and clone #1 of pUNO1-OSMR and photographed at indicated time points. J, Representative images of 3D spheroid formation assay. HEYA8 cells stably overexpressed with pUNO1-OSMR were cultured in Clona Cell media on low adherent plate and photographed at indicated days. Scale bar, 500 μm. Student's t test (two tailed, unpaired) was performed in (F, H). Data represent means±SEM. ****P£ 0.0001, ***P£0.001

Particular non-limiting embodiments of the present invention will now be described with reference to accompanying drawings.

### DETAILED DESCRIPTION

According to the present disclosure, it is now known that interleukin-6 signal transducer (hereinafter, “IL6ST”) and its dimerizing partner OSMR are two of the most highly-expressed receptor proteins in ovarian cancer cells. Also, according to the present disclosure, it is now known that OSMR is highly expressed in ovarian cancer cells, cancer-associated fibroblasts and endothelial cells. The inventors also found that oncostatin M (hereinafter, “OSM”), which is the ligand of OSMR, is produced by tumor-associated macrophages. Surprisingly, fully human monoclonal antibodies described herein have been found to be directed to the extracellular domain of OSMR that abrogated OSM-induced OSMR-IL6ST heterodimerization, promoted the internalization and degradation of OSMR, and effectively blocked OSMR-mediated signaling in vitro. The results disclosed herein surprisingly show that anti-OSMR antibody can mediate disruption of OSM-induced OSMR-IL6ST dimerization and oncogenic signaling.

### Antibodies of the Embodiments

In certain embodiments, an antibody or a fragment thereof that binds to at least a portion of OSMR protein and inhibits OSMR signaling and cancer cell proliferation are contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the anti-OSMR antibody is a monoclonal antibody or a humanized antibody.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to PD1. Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the V_{H} and CH1 domains; an Fv fragment consisting of the V_{L} and V_{H} domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. When choosing FR to flank CDRs, for example when humanizing or optimizing an antibody, FRs from antibodies which contain CDR sequences in the same canonical class are preferred.

As used herein the term “conservative substitution” refers to replacement of an amino acid with another amino acid which does not significantly deleteriously change the functional activity. A preferred example of a “conservative substitution” is the replacement of one amino acid with another amino acid which has a value .gtoreq.0 in a BLOSUM 62 substitution matrix (see Henikoff & Henikoff, 1992, PNAS 89:10915-10919).

Thus, by known means and as described herein, polyclonal or monoclonal antibodies, antibody fragments, and binding domains and CDRs (including engineered forms of any of the foregoing) may be created that are specific to OSMR protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, CL, and CH1 domains; (ii) the “Fd” fragment consisting of the V_{H} and CH1 domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see, for example, U.S. Pat. No. 5,091,513); and (ix) diabodies, multivalent or multispecific fragments constructed by gene fusion (see, for example, US Patent App. Pub. No. 20050214860). Fv, scFv, or diabody molecules may be stabilized by the incorporation of disulphide bridges linking the V_{H} and V_{L} domains. Minibodies comprising a scFv joined to a CH3 domain may also be made (See, for example, Hu et al, 1996, “Minibody: A Novel Engineered Anti-Carcinoembryonic Antigen Antibody Fragment (Single-Chain Fv-CH3) Which Exhibits Rapid, High-Level Targeting of Xenografts”, Cancer Res. 56:3055-3061).

In another aspect, an antibody of the present disclosure may comprise an amino acid sequence that corresponds to a first, second, and/or third complementarity determining region (CDR) from the light variable and/or heavy variable chain of the B01, B02, B03, B04, B05, B06, B07, B08, B09, B010, B12, B13, B14, B16, B17, B18, B19, B21, H09, H10, H11, H12, H13, H14, H15, or H16 monoclonal antibodies disclosed and described herein.

In certain aspects, an isolated antibody of the present disclosure comprises CDR sequences at least 80%, 85%, 90%, 91%, 92%, 93%, 94%, 95%, 96%, 97%, 98%, 99% or 100% identical to the CDR regions of the B01, B02, B03, B04, B05, B06, B07, B08, B09, B010, B12, B13, B14, B16, B17, B18, B19, B21, H09, H10, H11, H12, H13, H14, H15, or H16 heavy and light chain amino acid sequences. In further aspects, an antibody of the present disclosure comprises CDR regions identical to the B01, B02, B03, B04, B05, B06, B07, B08, B09, B010, B12, B13, B14, B16, B17, B18, B19, B21, H09, H10, H11, H12, H13, H14, H15, or H16 CDR regions, except for one or two amino acid substitutions, deletions, or insertions at one or more of the CDRs. For example, the antibody can comprise CDRs wherein the CDR sequences comprise 1 or 2 amino acid substitutions in the V_{H} CDR1, V_{H} CDR2, V_{H} CDR3, V_{H} CDR1, V_{H} CDR2 and/or V_{H} CDR3 relative to the CDRs of a B01, B02, B03, B04, B05, B06, B07, B08, B09, B010, B12, B13, B14, B16, B17, B18, B19, B21, H09, H10, H11, H12, H13, H14, H15, or H16 monoclonal antibody.

Thus, in some specific aspects, an antibody of the present disclosure comprises (a) a first V_{H} CDR at least 80%, 85%, 90%, 91%, 92%, 93%, 94%, 95%, 96%, 97%, 98%, 99% or 100% identical to V_{H} CDR1 of B01 (SEQ ID NO: 1), B02 (SEQ ID NO: 4), B03 (SEQ ID NO: 7), B04 (SEQ ID NO: 10), B05 (SEQ ID NO: 13), B06 (SEQ ID NO: 16), B07 (SEQ ID NO: 19), B08 (SEQ ID NO: 22), B09 (SEQ ID NO: 25), B10 (SEQ ID NO: 28), B12 (SEQ ID NO: 31), B13 (SEQ ID NO: 34), B14 (SEQ ID NO: 37), B16 (SEQ ID NO: 40), B17 (SEQ ID NO: 43), B18 (SEQ ID NO: 46), B19 (SEQ ID NO: 49), B21 (SEQ ID NO: 52), H09 (SEQ ID NO: 55), H10 (SEQ ID NO: 58), H11 (SEQ ID NO: 61), H12 (SEQ ID NO: 64), H13 (SEQ ID NO: 67), H14 (SEQ ID NO: 70), H15 (SEQ ID NO: 73), or H16 (SEQ ID NO: 76); (b) a second V_{H} CDR at least 80%, 85%, 90%, 91%, 92%, 93%, 94%, 95%, 96%, 97%, 98%, 99% or 100% identical to V_{H} CDR2 of B01 (SEQ ID NO: 2), B02 (SEQ ID NO: 5), B03 (SEQ ID NO: 8), B04 (SEQ ID NO: 11), B05 (SEQ ID NO: 14), B06 (SEQ ID NO: 17), B07 (SEQ ID NO: 20), B08 (SEQ ID NO: 23), B09 (SEQ ID NO: 26), B10 (SEQ ID NO: 29), B12 (SEQ ID NO: 32), B13 (SEQ ID NO: 35), B14 (SEQ ID NO: 38), B16 (SEQ ID NO: 41), B17 (SEQ ID NO: 44), B18 (SEQ ID NO: 47), B19 (SEQ ID NO: 50), B21 (SEQ ID NO: 53), H09 (SEQ ID NO: 56), H10 (SEQ ID NO: 59), H11 (SEQ ID NO: 62), H12 (SEQ ID NO: 65), H13 (SEQ ID NO: 68), H14 (SEQ ID NO: 71), H15 (SEQ ID NO: 74), or H16 (SEQ ID NO: 77); (c) a third V_{H} CDR at least 80%, 85%, 90%, 91%, 92%, 93%, 94%, 95%, 96%, 97%, 98%, 99% or 100% identical to V_{H} CDR3 of B01 (SEQ ID NO: 3), B02 (SEQ ID NO: 6), B03 (SEQ ID NO: 9), B04 (SEQ ID NO: 12), B05 (SEQ ID NO: 15), B06 (SEQ ID NO: 18), B07 (SEQ ID NO: 21), B08 (SEQ ID NO: 24), B09 (SEQ ID NO: 27), B10 (SEQ ID NO: 30), B12 (SEQ ID NO: 33), B13 (SEQ ID NO: 36), B14 (SEQ ID NO: 39), B16 (SEQ ID NO: 42), B17 (SEQ ID NO: 45), B18 (SEQ ID NO: 48), B19 (SEQ ID NO: 51), B21 (SEQ ID NO: 54), H09 (SEQ ID NO: 57), H10 (SEQ ID NO: 60), H11 (SEQ ID NO: 63), H12 (SEQ ID NO: 66), H13 (SEQ ID NO: 69), H14 (SEQ ID NO: 72), H15 (SEQ ID NO: 75), or H16 (SEQ ID NO: 78); (d) a first V_{L} CDR at least 80%, 85%, 90%, 91%, 92%, 93%, 94%, 95%, 96%, 97%, 98%, 99% or 100% identical to V_{L} CDR1 of B01 (SEQ ID NO: 79), B02 (SEQ ID NO: 81), B03 (SEQ ID NO: 83), B04 (SEQ ID NO: 85), B05 (SEQ ID NO: 87), B06 (SEQ ID NO: 89), B07 (SEQ ID NO: 91), B08 (SEQ ID NO: 93), B09 (SEQ ID NO: 95), B10 (SEQ ID NO: 97), B12 (SEQ ID NO: 99), B13 (SEQ ID NO: 101), B14 (SEQ ID NO: 103), B16 (SEQ ID NO: 105), B17 (SEQ ID NO: 107), B18 (SEQ ID NO: 109), B19 (SEQ ID NO: 111), B21 (SEQ ID NO: 113), H09 (SEQ ID NO: 115), H10 (SEQ ID NO: 117), H11 (SEQ ID NO: 119), H12 (SEQ ID NO: 121), H13 (SEQ ID NO: 123), H14 (SEQ ID NO: 125), H15 (SEQ ID NO: 127), or H16 (SEQ ID NO: 129); (e) a second V_{L} CDR at least 80%, 85%, 90%, 91%, 92%, 93%, 94%, 95%, 96%, 97%, 98%, 99% or 100% identical to V_{L} CDR2 selected from a group consisting of GNS, DNN, DAS, SHN, DAT, SNN, NNN, RNN, EDN, AAS, DVS, LGS, QDN, WDS, or PDC; and (f) a third V_{L} CDR at least 80%, 85%, 90%, 91%, 92%, 93%, 94%, 95%, 96%, 97%, 98%, 99% or 100% identical to V_{L} CDR3 of B01 (SEQ ID NO: 80), B02 (SEQ ID NO: 82), B03 (SEQ ID NO: 84), B04 (SEQ ID NO: 86), B05 (SEQ ID NO: 88), B06 (SEQ ID NO: 90), B07 (SEQ ID NO: 92), B08 (SEQ ID NO: 94), B09 (SEQ ID NO: 96), B10 (SEQ ID NO: 98), B12 (SEQ ID NO: 100), B13 (SEQ ID NO: 102), B14 (SEQ ID NO: 104), B16 (SEQ ID NO: 106), B17 (SEQ ID NO: 108), B18 (SEQ ID NO: 110), B19 (SEQ ID NO: 112), B21 (SEQ ID NO: 114), H09 (SEQ ID NO: 116), H10 (SEQ ID NO: 118), H11 (SEQ ID NO: 120), H12 (SEQ ID NO: 122), H13 (SEQ ID NO: 124), H14 (SEQ ID NO: 126), H15 (SEQ ID NO: 128), or H16 (SEQ ID NO: 130). In certain aspects, such an antibody is a humanized or de-immunized antibody comprising the foregoing CDRs on a human IgGs (e.g., IgG1, IgG2, IgG4, or a genetically modified IgG) backbone.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of the monoclonal antibody B01, which are represented by SEQ ID NOs: 1, 2, 3, 79, 80, and 81, respectively. In one aspect, the isolated antibody comprises CDR sequences that are identical to the CDR sequences of monoclonal antibody B01.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B01 (SEQ ID NO: 131) or the humanized V_{H} domain of B01 mAb; and a V_{L} domain at least about 80%, 85%, 90%, 91%, 92%, 93%, 94%, 95%, 96%, 97%, 98%, 99% or 100% identical to the V_{L} domain of B01 (SEQ ID NO: 157) or the humanized V_{L} domain of B01 mAb. For example, the antibody can comprise a V_{H} domain at least 95% identical to the V_{H} domain of the humanized B01 mAb and a V_{L} domain at least 95% identical to the V_{L} domain of the humanized B01 mAb. Thus, in some aspects, an antibody of the present disclosure can comprise a V_{H} domain identical to the V_{H} domain of humanized B01 mAb and a V_{L} domain identical to the V_{L} domain of the humanized B01 mAb. In a specific example, the isolated antibody comprises V_{H} and V_{L} domains identical to those of monoclonal antibody B01.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B02, which are represented by SEQ ID NOs: 4, 5, and 6; and SEQ ID NO: 81, the tripeptide DNN, and SEQ ID NO: 82; respectively. In one aspect, the isolated antibody comprises CDR sequences that are identical to the CDR sequences of monoclonal antibody B02.

In another aspect, the isolated antibody of the present disclosure can comprise a V_{H} domain at least about 80%, 85%, 90%, 91%, 92%, 93%, 94%, 95%, 96%, 97%, 98%, 99% or 100% identical to the V_{H} domain of B02 (SEQ ID NO: 132) or the humanized V_{H} domain of B02 mAb; and a V_{L} domain at least about 80%, 85%, 90%, 91%, 92%, 93%, 94%, 95%, 96%, 97%, 98%, 99% or 100% identical to the V_{L} domain of B02 (SEQ ID NO: 158) or the humanized V_{L} domain of B02 mAb. For example, the antibody can comprise a V_{H} domain at least 95% identical to the V_{H} domain of the humanized B02 mAb and a V_{L} domain at least 95% identical to the V_{L} domain of the humanized B02 mAb. Thus, in some aspects, an antibody can comprise a V_{H} domain identical to the V_{H} domain of humanized B02 mAb and a V_{L} domain identical to the V_{L} domain of the humanized B02 mAb. In a specific example, the isolated antibody comprises V_{H} and V_{L} domains identical to those of monoclonal antibody B02.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B03, which are represented by SEQ ID NOs: 7, 8, 9; and SEQ ID NO: 83, the tripeptide DAS, and SEQ ID NO: 84; 85, 86, and 87, respectively. In one aspect, the isolated antibody comprises CDR sequences that are identical to the CDR sequences of monoclonal antibody B03.

In another aspect, the isolated antibody of the present disclosure can comprise a V_{H} domain at least about 80%, 85%, 90%, 91%, 92%, 93%, 94%, 95%, 96%, 97%, 98%, 99% or 100% identical to the V_{H} domain of B03 (SEQ ID NO: 133) or the humanized V_{H} domain of B03 mAb; and a V_{L} domain at least about 80%, 85%, 90%, 91%, 92%, 93%, 94%, 95%, 96%, 97%, 98%, 99% or 100% identical to the V_{L} domain of B03 (SEQ ID NO: 159) or the humanized V_{L} domain of B03 mAb. For example, the antibody can comprise a V_{H} domain at least 95% identical to the V_{H} domain of the humanized B03 mAb and a V_{L} domain at least 95% identical to the V_{L} domain of the humanized B03 mAb. Thus, in some aspects, an antibody can comprise a V_{H} domain identical to the V_{H} domain of humanized B03 mAb and a V_{L} domain identical to the V_{L} domain of the humanized B03 mAb. In a specific example, the isolated antibody comprises V_{H} and V_{L} domains identical to those of monoclonal antibody B03.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B04, which are represented by SEQ ID NOs: 10, 11, 12; and SEQ ID NO: 85, the tripeptide DAS, and SEQ ID NO: 86; respectively. In one aspect, the isolated antibody comprises CDR sequences that are identical to the CDR sequences of monoclonal antibody B04.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B04 (SEQ ID NO: 134) or the humanized V_{H} domain of B04 mAb; and a V_{L} domain at least about 80%, 85%, 90%, 91%, 92%, 93%, 94%, 95%, 96%, 97%, 98%, 99% or 100% identical to the V_{L} domain of B04 (SEQ ID NO: 160) or the humanized V_{L} domain of B04 mAb. For example, the antibody can comprise a V_{H} domain at least 95% identical to the V_{H} domain of the humanized B04 mAb and a V_{L} domain at least 95% identical to the VI domain of the humanized B04 mAb. Thus, in some aspects, an antibody can comprise a V_{H} domain identical to the V_{H} domain of humanized B04 mAb and a V_{L} domain identical to the V_{L} domain of the humanized B04 mAb. In a specific example, the isolated antibody can comprise V_{H} and V_{L} domains identical to those of monoclonal antibody B04.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B05, which are represented by SEQ ID NOs: 13, 14, 15; and SEQ ID NO: 87, the tripeptide DAS, and SEQ ID NO: 88; respectively. In one aspect, the isolated antibody comprises CDR sequences that are identical to the CDR sequences of monoclonal antibody B05.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B05 (SEQ ID NO: 135) or the humanized V_{H} domain of B05 mAb; and a V_{L} domain at least about 80%, 85%, 90%, 91%, 92%, 93%, 94%, 95%, 96%, 97%, 98%, 99% or 100% identical to the V_{L} domain of B05 (SEQ ID NO: 161) or the humanized V_{L} domain of B05 mAb. For example, the antibody can comprise a V_{H} domain at least 95% identical to the V_{H} domain of the humanized B05 mAb and a V_{L} domain at least 95% identical to the V_{L} domain of the humanized B05 mAb. Thus, in some aspects, an antibody can comprise a V_{H} domain identical to the V_{H} domain of humanized B05 mAb and a V_{L} domain identical to the V_{L} domain of the humanized B05 mAb. In a specific example, the isolated antibody can comprise V_{H} and V_{L} domains identical to those of monoclonal antibody B05.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B06, which are represented by SEQ ID NOs: 16, 17, 18; and SEQ ID NO: 89, the tripeptide SHN, and SEQ ID NO: 90; respectively. In one aspect, the isolated antibody comprises CDR sequences that are identical to the CDR sequences of monoclonal antibody B06.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B06 (SEQ ID NO: 136) or the humanized V_{H} domain of B06 mAb; and a V_{L} domain at least about 80%, 85%, 90%, 91%, 92%, 93%, 94%, 95%, 96%, 97%, 98%, 99% or 100% identical to the V_{L} domain of B06 (SEQ ID NO: 162) or the humanized V_{L} domain of B06 mAb. For example, the antibody can comprise a V_{H} domain at least 95% identical to the V_{H} domain of the humanized B06 mAb and a V_{L} domain at least 95% identical to the V_{L} domain of the humanized B06 mAb. Thus, in some aspects, the antibody can comprise a V_{H} domain identical to the V_{H} domain of humanized B06 mAb and a V_{L} domain identical to the V_{L} domain of the humanized B06 mAb. In a specific example, the isolated antibody can comprise V_{H} and V_{L} domains identical to those of monoclonal antibody B06.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B07, which are represented by SEQ ID NOs: 19, 20, 21; and SEQ ID NO: 91, the tripeptide DAT, and SEQ ID NO: 92; respectively. In one aspect, the isolated antibody comprises CDR sequences that are identical to the CDR sequences of monoclonal antibody B07.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B07 (SEQ ID NO: 137) or the humanized V_{H} domain of B07 mAb; and a V_{L} domain at least about 80%, 85%, 90%, 91%, 92%, 93%, 94%, 95%, 96%, 97%, 98%, 99% or 100% identical to the V_{L} domain of B07 (SEQ ID NO: 163) or the humanized V_{L} domain of B07 mAb. For example, the antibody can comprise a V_{H} domain at least 95% identical to the V_{H} domain of the humanized B07 mAb and a V_{L} domain at least 95% identical to the V_{L} domain of the humanized B07 mAb. Thus, in some aspects, the antibody can comprise a V_{H} domain identical to the V_{H} domain of humanized B07 mAb and a V_{L} domain identical to the V_{L} domain of the humanized B07 mAb. In a specific example, the isolated antibody can comprise V_{H} and V_{L} domains identical to those of monoclonal antibody B07.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B08, which are represented by SEQ ID NOs: 22, 23, 24; and SEQ ID NO: 93, the tripeptide SNN, and SEQ ID NO: 94; respectively. In one aspect, the isolated antibody comprises CDR sequences that are identical to the CDR sequences of monoclonal antibody B08.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B08 (SEQ ID NO: 138) or the humanized V_{H} domain of B08 mAb; and a V_{L} domain at least about 80%, 85%, 90%, 91%, 92%, 93%, 94%, 95%, 96%, 97%, 98%, 99% or 100% identical to the V_{L} domain of B08 (SEQ ID NO: 164) or the humanized V_{L} domain of B08 mAb. For example, the antibody can comprise a V_{H} domain at least 95% identical to the V_{H} domain of the humanized B08 mAb and a V_{L} domain at least 95% identical to the V_{L} domain of the humanized B08 mAb. Thus, in some aspects, an antibody can comprise a V_{H} domain identical to the V_{H} domain of humanized B08 mAb and a V_{L} domain identical to the V_{L} domain of the humanized B08 mAb. In a specific example, the isolated antibody can comprise V_{H} and V_{L} domains identical to those of monoclonal antibody B08.

In further aspects, and isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B09, which are represented by SEQ ID NOs: 25, 26, 27; and SEQ ID NO: 95, the tripeptide NNN, and SEQ ID NO: 96; respectively. In one aspect, the isolated antibody comprises CDR sequences that are identical to the CDR sequences of monoclonal antibody B09.

In another aspect, the isolated antibody can comprise a V_{H} domain at least about 80%, 85%, 90%, 91%, 92%, 93%, 94%, 95%, 96%, 97%, 98%, 99% or 100% identical to the V_{H} domain of B09 (SEQ ID NO: 139) or the humanized V_{H} domain of B09 mAb; and a V_{L} domain at least about 80%, 85%, 90%, 91%, 92%, 93%, 94%, 95%, 96%, 97%, 98%, 99% or 100% identical to the V_{L} domain of B09 (SEQ ID NO: 165) or the humanized V_{L} domain of B09 mAb. For example, the antibody can comprise a V_{H} domain at least 95% identical to the V_{H} domain of the humanized B09 mAb and a V_{L} domain at least 95% identical to the V_{L} domain of the humanized B09 mAb. Thus, in some aspects, an antibody can comprise a V_{H} domain identical to the V_{H} domain of humanized B09 mAb and a V_{L} domain identical to the V_{L} domain of the humanized B09 mAb. In a specific example, the isolated antibody comprises V_{H} and V_{L} domains identical to those of monoclonal antibody B09.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B010, which are represented by SEQ ID NOs: 28, 29, 30; and SEQ ID NO: 97, the tripeptide RNN, and SEQ ID NO: 98; respectively. In one aspect, the isolated antibody comprises CDR sequences that are identical to the CDR sequences of monoclonal antibody B010.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B010 (SEQ ID NO: 140) or the humanized V_{H} domain of B10 mAb; and a V_{L} domain at least about 80%, 85%, 90%, 91%, 92%, 93%, 94%, 95%, 96%, 97%, 98%, 99% or 100% identical to the V_{L} domain of B10 (SEQ ID NO: 166) or the humanized V_{L} domain of B10 mAb. For example, the antibody can comprise a V_{H} domain at least 95% identical to the V_{H} domain of the humanized B10 mAb and a V_{L} domain at least 95% identical to the V_{L} domain of the humanized B10 mAb. Thus, in some aspects, an antibody comprises a V_{H} domain identical to the V_{H} domain of humanized B10 mAb and a V_{L} domain identical to the V_{L} domain of the humanized B10 mAb. In a specific example, the isolated antibody can comprise V_{H} and V_{L} domains identical to those of monoclonal antibody B10.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B12, which are represented by SEQ ID NOs: 31, 32, 33; and SEQ ID NO: 99, the tripeptide EDN, and SEQ ID NO: 100; respectively. In one aspect, the isolated antibody comprises CDR sequences that are identical to the CDR sequences of monoclonal antibody B12.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B12 (SEQ ID NO: 141) or the humanized V_{H} domain of B12 mAb; and a V_{L} domain at least about 80%, 85%, 90%, 91%, 92%, 93%, 94%, 95%, 96%, 97%, 98%, 99% or 100% identical to the V_{L} domain of B12 (SEQ ID NO: 167) or the humanized V_{L} domain of B12 mAb. For example, the antibody can comprise a V_{H} domain at least 95% identical to the V_{H} domain of the humanized B12 mAb and a V_{L} domain at least 95% identical to the V_{L} domain of the humanized B12 mAb. Thus, in some aspects, an antibody comprises a V_{H} domain identical to the V_{H} domain of humanized B12 mAb and a V_{L} domain identical to the V_{L} domain of the humanized B12 mAb. In a specific example, the isolated antibody can comprise V_{H} and V_{L} domains identical to those of monoclonal antibody B12.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B13, which are represented by SEQ ID NOs: 34, 35, 36; and SEQ ID NO: 101, the tripeptide SNN, and SEQ ID NO: 102; respectively. In one aspect, the isolated antibody comprises CDR sequences that are identical to the CDR sequences of monoclonal antibody B13.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B13 (SEQ ID NO: 142) or the humanized V_{H} domain of B13 mAb; and a V_{L} domain at least about 80%, 85%, 90%, 91%, 92%, 93%, 94%, 95%, 96%, 97%, 98%, 99% or 100% identical to the V_{L} domain of B13 (SEQ ID NO: 168) or the humanized V_{L} domain of B13 mAb. For example, the antibody can comprise a V_{H} domain at least 95% identical to the V_{H} domain of the humanized B13 mAb and a V_{L} domain at least 95% identical to the V_{L} domain of the humanized B13 mAb. Thus, in some aspects, an antibody comprises a V_{H} domain identical to the V_{H} domain of humanized B13 mAb and a V_{L} domain identical to the V_{L} domain of the humanized B13 mAb. In a specific example, the isolated antibody can comprise V_{H} and V_{L} domains identical to those of monoclonal antibody B13.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B14, which are represented by SEQ ID NOs: 37, 38, 39; and SEQ ID NO: 103, the tripeptide AAS, and SEQ ID NO: 104; respectively. In one aspect, the isolated antibody comprises CDR sequences that are identical to the CDR sequences of monoclonal antibody B14.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B14 (SEQ ID NO: 143) or the humanized V_{H} domain of B14 mAb; and a V_{L} domain at least about 80%, 85%, 90%, 91%, 92%, 93%, 94%, 95%, 96%, 97%, 98%, 99% or 100% identical to the V_{L} domain of B14 (SEQ ID NO: 169) or the humanized V_{L} domain of B14 mAb. For example, the antibody can comprise a V_{H} domain at least 95% identical to the V_{H} domain of the humanized B14 mAb and a V_{L} domain at least 95% identical to the V_{L} domain of the humanized B14 mAb. Thus, in some aspects, an antibody comprises a V_{H} domain identical to the V_{H} domain of humanized B14 mAb and a V_{L} domain identical to the V_{L} domain of the humanized B14 mAb. In a specific example, the isolated antibody can comprise V_{H} and V_{L} domains identical to those of monoclonal antibody B14.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B016, which are represented by SEQ ID NOs: 40, 41, 42; and SEQ ID NO: 105, the tripeptide DAS, and SEQ ID NO: 106; respectively. In one aspect, the isolated antibody comprises CDR sequences that are identical to the CDR sequences of monoclonal antibody B16.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B16 (SEQ ID NO: 144) or the humanized V_{H} domain of B16 mAb; and a V_{L} domain at least about 80%, 85%, 90%, 91%, 92%, 93%, 94%, 95%, 96%, 97%, 98%, 99% or 100% identical to the V_{L} domain of B16 (SEQ ID NO: 170) or the humanized V_{L} domain of B16 mAb. For example, the antibody can comprise a V_{H} domain at least 95% identical to the V_{H} domain of the humanized B16 mAb and a V_{L} domain at least 95% identical to the V_{L} domain of the humanized B16 mAb. Thus, in some aspects, an antibody comprises a V_{H} domain identical to the V_{H} domain of humanized B16 mAb and a V_{L} domain identical to the V_{L} domain of the humanized B16 mAb. In a specific example, the isolated antibody can comprise V_{H} and V_{L} domains identical to those of monoclonal antibody B16.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B17, which are represented by SEQ ID NOs: 43, 44, 45; and SEQ ID NO: 107, the tripeptide DVS, and SEQ ID NO: 108; respectively. In one aspect, the isolated antibody comprises CDR sequences that are identical to the CDR sequences of monoclonal antibody B17.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B17 (SEQ ID NO: 145) or the humanized V_{H} domain of B17 mAb; and a V_{L} domain at least about 80%, 85%, 90%, 91%, 92%, 93%, 94%, 95%, 96%, 97%, 98%, 99% or 100% identical to the V_{L} domain of B17 (SEQ ID NO: 171) or the humanized V_{L} domain of B17 mAb. For example, the antibody can comprise a V_{H} domain at least 95% identical to the V_{H} domain of the humanized B17 mAb and a V_{L} domain at least 95% identical to the V_{L} domain of the humanized B17 mAb. Thus, in some aspects, an antibody comprises a V_{H} domain identical to the V_{H} domain of humanized B17 mAb and a V_{L} domain identical to the V_{L} domain of the humanized B17 mAb. In a specific example, the isolated antibody can comprise V_{H} and V_{L} domains identical to those of monoclonal antibody B17.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B18, which are represented by SEQ ID NOs: 46, 47, 48; and SEQ ID NO: 109, the tripeptide LGS, and SEQ ID NO: 110; respectively. In one aspect, the isolated antibody comprises CDR sequences that are identical to the CDR sequences of monoclonal antibody B18.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B18 (SEQ ID NO: 146) or the humanized V_{H} domain of B18 mAb; and a V_{L} domain at least about 80%, 85%, 90%, 91%, 92%, 93%, 94%, 95%, 96%, 97%, 98%, 99% or 100% identical to the V_{L} domain of B18 (SEQ ID NO: 172) or the humanized V_{L} domain of B18 mAb. For example, the antibody can comprise a V_{H} domain at least 95% identical to the V_{H} domain of the humanized B18 mAb and a V_{L} domain at least 95% identical to the V_{L} domain of the humanized B18 mAb. Thus, in some aspects, an antibody comprises a V_{H} domain identical to the V_{H} domain of humanized B18 mAb and a V_{L} domain identical to the V_{L} domain of the humanized B18 mAb. In a specific example, the isolated antibody can comprise V_{H} and V_{L} domains identical to those of monoclonal antibody B18.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B19, which are represented by SEQ ID NOs: 49, 50, 51; and SEQ ID NO: 111, the tripeptide SNN, and SEQ ID NO: 112; respectively. In one aspect, the isolated antibody comprises CDR sequences that are identical to the CDR sequences of monoclonal antibody B19.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B19 (SEQ ID NO: 147) or the humanized V_{H} domain of B19 mAb; and a V_{L} domain at least about 80%, 85%, 90%, 91%, 92%, 93%, 94%, 95%, 96%, 97%, 98%, 99% or 100% identical to the V_{L} domain of B19 (SEQ ID NO: 173) or the humanized V_{L} domain of B19 mAb. For example, the antibody can comprise a V_{H} domain at least 95% identical to the V_{H} domain of the humanized B19 mAb and a V_{L} domain at least 95% identical to the V_{L} domain of the humanized B19 mAb. Thus, in some aspects, an antibody comprises a V_{H} domain identical to the V_{H} domain of humanized B19 mAb and a V_{L} domain identical to the V_{L} domain of the humanized B19 mAb. In a specific example, the isolated antibody can comprise V_{H} and V_{L} domains identical to those of monoclonal antibody B19.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody B21, which are represented by SEQ ID NOs: 52, 53, 54; and SEQ ID NO: 113, the tripeptide AAS, and SEQ ID NO: 114; respectively. In one aspect, the isolated antibody comprises CDR sequences that are identical to the CDR sequences of monoclonal antibody B21.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of B21 (SEQ ID NO: 148) or the humanized V_{H} domain of B21 mAb; and a V_{L} domain at least about 80%, 85%, 90%, 91%, 92%, 93%, 94%, 95%, 96%, 97%, 98%, 99% or 100% identical to the V_{L} domain of B21 (SEQ ID NO: 174) or the humanized V_{L} domain of B21 mAb. For example, the antibody can comprise a V_{H} domain at least 95% identical to the V_{H} domain of the humanized B21 mAb and a V_{L} domain at least 95% identical to the V_{L} domain of the humanized B21 mAb. Thus, in some aspects, an antibody comprises a V_{H} domain identical to the V_{H} domain of humanized B21 mAb and a V_{L} domain identical to the V_{L} domain of the humanized B21 mAb. In a specific example, the isolated antibody can comprise V_{H} and V_{L} domains identical to those of monoclonal antibody B21.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H09, which are represented by SEQ ID NOs: 55, 56, 57; and SEQ ID NO: 115, the tripeptide SNN, and SEQ ID NO: 116; respectively. In one aspect, the isolated antibody comprises CDR sequences that are identical to the CDR sequences of monoclonal antibody H09.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H09 (SEQ ID NO: 149) or the humanized V_{H} domain of H09 mAb; and a V_{L} domain at least about 80%, 85%, 90%, 91%, 92%, 93%, 94%, 95%, 96%, 97%, 98%, 99% or 100% identical to the V_{L} domain of H09 (SEQ ID NO: 175) or the humanized V_{L} domain of H09 mAb. For example, the antibody can comprise a V_{H} domain at least 95% identical to the V_{H} domain of the humanized H09 mAb and a V_{L} domain at least 95% identical to the V_{L} domain of the humanized H09 mAb. Thus, in some aspects, an antibody comprises a V_{H} domain identical to the V_{H} domain of humanized H09 mAb and a V_{L} domain identical to the V_{L} domain of the humanized H09 mAb. In a specific example, the isolated antibody can comprise V_{H} and V_{L} domains identical to those of monoclonal antibody H09.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H10, which are represented by SEQ ID NOs: 58., 59, 60; and SEQ ID NO: 117, the tripeptide SNN, and SEQ ID NO: 118; respectively. In one aspect, the isolated antibody comprises CDR sequences that are identical to the CDR sequences of monoclonal antibody H10.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H10 (SEQ ID NO: 150) or the humanized V_{H} domain of H10 mAb; and a V_{L} domain at least about 80%, 85%, 90%, 91%, 92%, 93%, 94%, 95%, 96%, 97%, 98%, 99% or 100% identical to the V_{L} domain of H10 (SEQ ID NO: 176) or the humanized V_{L} domain of H10 mAb. For example, the antibody can comprise a V_{H} domain at least 95% identical to the V_{H} domain of the humanized H10 mAb and a V_{L} domain at least 95% identical to the V_{L} domain of the humanized H10 mAb. Thus, in some aspects, an antibody comprises a V_{H} domain identical to the V_{H} domain of humanized H10 mAb and a V_{L} domain identical to the V_{L} domain of the humanized H10 mAb. In a specific example, the isolated antibody can comprise V_{H} and V_{L} domains identical to those of monoclonal antibody H10.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H11, which are represented by SEQ ID NOs: 61, 62, 63; and SEQ ID NO: 119, the tripeptide QDN, and SEQ ID NO: 120; respectively. In one aspect, the isolated antibody comprises CDR sequences that are identical to the CDR sequences of monoclonal antibody H11.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H11 (SEQ ID NO: 151) or the humanized V_{H} domain of H11 mAb; and a V_{L} domain at least about 80%, 85%, 90%, 91%, 92%, 93%, 94%, 95%, 96%, 97%, 98%, 99% or 100% identical to the V_{L} domain of H11 (SEQ ID NO: 177) or the humanized V_{L} domain of H11 mAb. For example, the antibody can comprise a V_{H} domain at least 95% identical to the V_{H} domain of the humanized H11 mAb and a V_{L} domain at least 95% identical to the V_{L} domain of the humanized H11 mAb. Thus, in some aspects, an antibody comprises a V_{H} domain identical to the V_{H} domain of humanized H11 mAb and a V_{L} domain identical to the V_{L} domain of the humanized H11 mAb. In a specific example, the isolated antibody can comprise V_{H} and V_{L} domains identical to those of monoclonal antibody H11.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H12, which are represented by SEQ ID NOs: 64, 65, 66; and SEQ ID NO: 121, the tripeptide WDS, and SEQ ID NO: 122; respectively. In one aspect, the isolated antibody comprises CDR sequences that are identical to the CDR sequences of monoclonal antibody H12.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H12 (SEQ ID NO: 152) or the humanized V_{H} domain of H12 mAb; and a V_{L} domain at least about 80%, 85%, 90%, 91%, 92%, 93%, 94%, 95%, 96%, 97%, 98%, 99% or 100% identical to the V_{L} domain of H12 (SEQ ID NO: 178) or the humanized V_{L} domain of H12 mAb. For example, the antibody can comprise a V_{H} domain at least 95% identical to the V_{H} domain of the humanized H12 mAb and a V_{L} domain at least 95% identical to the V_{L} domain of the humanized H12 mAb. Thus, in some aspects, an antibody comprises a V_{H} domain identical to the V_{H} domain of humanized H12 mAb and a V_{L} domain identical to the V_{L} domain of the humanized H12 mAb. In a specific example, the isolated antibody can comprise V_{H} and V_{L} domains identical to those of monoclonal antibody H12.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H13, which are represented by SEQ ID NOs: 67, 68, 69; and SEQ ID NO: 123, the tripeptide EDN, and SEQ ID NO: 124; respectively. In one aspect, the isolated antibody comprises CDR sequences that are identical to the CDR sequences of monoclonal antibody H13.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H13 (SEQ ID NO: 153) or the humanized V_{H} domain of H13 mAb; and a V_{L} domain at least about 80%, 85%, 90%, 91%, 92%, 93%, 94%, 95%, 96%, 97%, 98%, 99% or 100% identical to the V_{L} domain of H13 (SEQ ID NO: 179) or the humanized V_{L} domain of H13 mAb. For example, the antibody can comprise a V_{H} domain at least 95% identical to the V_{H} domain of the humanized H13 mAb and a V_{L} domain at least 95% identical to the V_{L} domain of the humanized H13 mAb. Thus, in some aspects, an antibody comprises a V_{H} domain identical to the V_{H} domain of humanized H13 mAb and a V_{L} domain identical to the V_{L} domain of the humanized H13 mAb. In a specific example, the isolated antibody can comprise V_{H} and V_{L} domains identical to those of monoclonal antibody H13.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H14, which are represented by SEQ ID NOs: 70, 71, 72; and SEQ ID NO: 125, the tripeptide SNN, and SEQ ID NO: 126; respectively. In one aspect, the isolated antibody comprises CDR sequences that are identical to the CDR sequences of monoclonal antibody H14.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H14 (SEQ ID NO: 154) or the humanized V_{H} domain of H14 mAb; and a V_{L} domain at least about 80%, 85%, 90%, 91%, 92%, 93%, 94%, 95%, 96%, 97%, 98%, 99% or 100% identical to the V_{L} domain of H14 (SEQ ID NO: 180) or the humanized V_{L} domain of H14 mAb. For example, the antibody can comprise a V_{H} domain at least 95% identical to the V_{H} domain of the humanized H14 mAb and a V_{L} domain at least 95% identical to the V_{L} domain of the humanized H14 mAb. Thus, in some aspects, an antibody comprises a V_{H} domain identical to the V_{H} domain of humanized H14 mAb and a V_{L} domain identical to the V_{L} domain of the humanized H14 mAb. In a specific example, the isolated antibody can comprise V_{H} and V_{L} domains identical to those of monoclonal antibody H14.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H15, which are represented by SEQ ID NOs: 73, 74, 75; and SEQ ID NO: 127, the tripeptide PDC, and SEQ ID NO: 128; respectively. In one aspect, the isolated antibody comprises CDR sequences that are identical to the CDR sequences of monoclonal antibody H15.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H15 (SEQ ID NO: 155) or the humanized V_{H} domain of H15 mAb; and a V_{L} domain at least about 80%, 85%, 90%, 91%, 92%, 93%, 94%, 95%, 96%, 97%, 98%, 99% or 100% identical to the V_{L} domain of H15 (SEQ ID NO: 181) or the humanized V_{L} domain of H15 mAb. For example, the antibody can comprise a V_{H} domain at least 95% identical to the V_{H} domain of the humanized H15 mAb and a V_{L} domain at least 95% identical to the V_{L} domain of the humanized H15 mAb. Thus, in some aspects, an antibody comprises a V_{H} domain identical to the V_{H} domain of humanized H15 mAb and a V_{L} domain identical to the V_{L} domain of the humanized H15 mAb. In a specific example, the isolated antibody can comprise V_{H} and V_{L} domains identical to those of monoclonal antibody H15.

In further aspects, an isolated antibody of the present disclosure can comprise a first V_{H}, a second V_{H}, a third V_{H}, a first V_{L}, a second V_{L}, and a third V_{L} CDR sequence at least 80%, 85%, 90%, 91%, 92%, 93%, 94%, 95%, 96%, 97%, 98%, 99% or 100% identical to the corresponding CDR sequence of monoclonal antibody H16, which are represented by SEQ ID NOs: 76, 77, 78; and SEQ ID NO: 129, the tripeptide AAS, and SEQ ID NO: 130; respectively. In one aspect, the isolated antibody comprises CDR sequences that are identical to the CDR sequences of monoclonal antibody H16.

In another aspect, the isolated antibody comprises a V_{H} domain at least about 80%, 85%, 90%, 91%, 92%, 93%, 94%, 95%, 96%, 97%, 98%, 99% or 100% identical to the V_{H} domain of H16 (SEQ ID NO: 156) or the humanized V_{H} domain of H16 mAb; and a V_{L} domain at least about 80%, 85%, 90%, 91%, 92%, 93%, 94%, 95%, 96%, 97%, 98%, 99% or 100% identical to the V_{L} domain of H16 (SEQ ID NO: 182) or the humanized V_{L} domain of H16 mAb. For example, the antibody can comprise a V_{H} domain at least 95% identical to the V_{H} domain of the humanized H16 mAb and a V_{L} domain at least 95% identical to the V_{L} domain of the humanized H16 mAb. Thus, in some aspects, an antibody comprises a V_{H} domain identical to the V_{H} domain of humanized H16 mAb and a V_{L} domain identical to the V_{L} domain of the humanized H16 mAb. In a specific example, the isolated antibody can comprise V_{H} and V_{L} domains identical to those of monoclonal antibody H16.

Antibody-like binding peptidomimetics are also contemplated in embodiments. Liu et al. (Murali, R.; Liu, Q.; Cheng, X.; Berezov, A.; Richter, M.; Furuchi, K., Greene, M. I.; Zhang, H. Antibody like peptidomimetics as large scale immunodetection probes. Cell. Mol. Biol. (Noisy-le-grand) 2003, 49:209-216) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as OSMR protein, in order to produce antibodies specific for OSMR protein. Frequently an antigen is bound or conjugated to another molecule to enhance the immune response. As used herein, a conjugate is any peptide, polypeptide, protein, or nonproteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody (or “mAb”) is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a EGFL6 antigen with an immortal myeloma cell (usually mouse myeloma). This technology provides a method to propagate a single antibody producing cell for an indefinite number of generations, such that unlimited quantities of structurally identical antibodies having the same antigen or epitope specificity (monoclonal antibodies) may be produced.

Plasma B cells (CD45+CD5-CD19+) may be isolated from freshly prepared rabbit peripheral blood mononuclear cells of immunized rabbits and further selected for OSMR binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. OSMR specific binding Fab may be selected out through multiple rounds enrichment panning and sequenced. Selected OSMR binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see, for example, U.S. Pat. Nos. 5,091,513 and 6,881,557). Without being limited by theory, it is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, CAR, and fully human, are well known in the art and highly predictable. For example, the following U.S. patents and patent applications, which are incorporated herein by reference in their entirety, provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546. These techniques are further described in: Marks (1992); Stemmer (1994); Gram et al. (1992); Barbas et al. (1994); and Schier et al. (1996).

It is fully expected that antibodies to OSMR will have the ability to neutralize or counteract the effects of OSMR regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins (e.g., monoclonal antibodies) of the present disclosure may be isolated (e.g., enriched and/or purified to some degree) and/or may be recombinant or synthesized in vitro. Alternatively, a nonrecombinant or recombinant protein may be isolated from bacteria. It is also contemplated that a bacteria containing such a variant may be implemented in compositions and methods. Consequently, a protein need not be isolated.

Thus, the present disclosure provides an isolated or recombinant monoclonal antibody that specifically binds to OSMR. In certain aspects, an antibody that competes for the binding of OSMR with the B01, B02, B03, B04, B05, B06, B07, B08, B09, B010, B12, B13, B14, B16, B17, B18, B19, B21, H09, H10, H11, H12, H13, H14, H15, or H16 monoclonal antibody (each disclosed and described herein) is provided. In certain aspects, the antibody may comprise all or part of the heavy chain variable region and/or light chain variable region of the B01, B02, B03, B04, B05, B06, B07, B08, B09, B010, B12, B13, B14, B16, B17, B18, B19, B21, H09, H10, H11, H12, H13, H14, H15, or H16 monoclonal antibodies.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 6 1, 62, 63, 64, 65, 66, 67, 68, 69, 70, 71, 72, 73, 74, 75, 76, 77, 78, 79, 80, 8 1, 82, 83, 84, 85, 86, 87, 88, 89, 90, 9 1, 92, 93, 94, 95, 96, 97, 98, 99, or 100% may be an antibody that binds OSMR.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against OSMR, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent a such diethylenetriaminepentaacetic acid anhydride (DTPA); ethylenetriaminetetraacetic acid; N-chloro-p-toluenesulfonamide; and/or tetrachloro-3α-6α-diphenylglycouril attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### Chimeric Antigen Receptors

As used herein the term “chimeric antigen receptor” or “CAR” refers to an artificially constructed hybrid protein or polypeptide containing an antigen-binding domain of an antibody (e.g., a single chain variable fragment (scFv)) linked to a domain or signaling, e.g., T-cell signaling or T-cell activation domains, that activates an immune cell, e.g., a T cell or a NK cell. CARs are capable of redirecting the immune cell specificity and reactivity toward a selected target in a non-MHC-restricted manner, taking advantage of the antigen-binding properties of monoclonal antibodies. This non-MHC-restricted antigen recognition confers on immune cells expressing CARs the ability to recognize an antigen independent of processing, thus bypassing a mechanism of tumor escape. In another aspect, provided is a chimeric antigen receptor (CAR) protein comprising an antigen-binding fragment as provided herein. In another aspect, provided is an isolated nucleic acid that encodes a CAR protein as provided herein.

In another aspect, an engineered cell comprising the isolated nucleic acid as provided herein. In certain embodiments, the engineered cell is a T cell, NK cell, or myeloid cell. In another aspect, the present disclosure provides immune cells which express a chimeric antigen receptor (CAR). In some embodiments, the CAR comprises an antigen-binding fragment provided herein. In some embodiments, the CAR protein includes from the N-terminus to the C-terminus: a leader peptide, an anti-OSMR heavy chain variable domain, a linker domain, an anti-OSMR light chain variable domain, a human IgG1-CH2-CH3 domain, a spacer region, a CD28 transmembrane domain, an anti-OSMR intracellular co-stimulatory signaling and a CD3 intracellular T cell signaling domain.

In certain embodiments, the chimeric antigen receptor comprising an antigen-binding domain at least 80%, 85%, 90%, 91%, 92%, 93%, 94%, 95%, 96%, 97%, 98%, 99% or 100% identical to the antigen-binding domain of any one of the OSMR-specific monoclonal antibodies disclosed herein. In certain embodiments, the engineered cell expresses an antigen-binding domain at least 80%, 85%, 90%, 91%, 92%, 93%, 94%, 95%, 96%, 97%, 98%, 99% or 100% identical to the antigen-binding domain of any one of the OSMR-specific monoclonal antibodies disclosed herein.

In some embodiments, provided is a method of treating or ameliorating the effect of a cancer or in a subject, the method comprising administering to the subject a therapeutically effective amount of the antibody or an antigen-binding fragment thereof as defined herein. In the treatment of cancer, the method may reduce or eradicate the tumor burden in the subject, may reduce the number of tumor cells, may reduce tumor size, may eradicate the tumor in the subject. In some embodiments, the cancer treated is ovarian cancer. In some embodiments, the immune cells can be genetically engineered to express antigen receptors such as engineered T cell receptors (TCRs) and/or chimeric antigen receptors (CARs). For example, the host cells (e.g., autologous or allogeneic T-cells) are modified to express a TCR having antigenic specificity for a cancer antigen. In particular embodiments, NK cells are engineered to express a TCR. The NK cells may be further engineered to express a CAR. Multiple CARs and/or TCRs, such as to different antigens, may be added to a single cell type, such as T cells or NK cells.

### Treatment of Diseases

Certain aspects of the present embodiments can be used to prevent or treat a disease or disorder associated with IL6/JAK/STAT signaling. Signaling mediated by OSMR may be reduced by any suitable drugs to prevent cancer cell proliferation. Preferably, such substances would be an anti-OSMR antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits the OSMR. signaling.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

### Pharmaceutical Preparations

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 g/kg/body weight to about 1000 mg/kg/body weight (this such range includes intervening doses) or more per administration, and any range derivable therein. In non-limiting examples of a derivable range from the numbers listed herein, a range of about 5 g/kg/body weight to about 100 mg/kg/body weight, about 5 g/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Combination Treatments

In certain embodiments, the compositions and methods of the present embodiments involve an antibody or an antibody fragment against OSMR to inhibit its activity in cancer cell proliferation, in combination with a second or additional therapy. Such therapy can be applied in the treatment of any disease that is associated with OSM-mediated cell proliferation. For example, the disease may be cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated, as necessary.

Various combinations may be employed. For the example below an antibody therapy is “A” and an anti-cancer therapy is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

Administration of any compound or therapy of the present embodiments to a patient will follow general protocols for the administration of such compounds, considering the toxicity, if any, of the agents. Therefore, in some embodiments there is a step of monitoring toxicity that is attributable to combination therapy.

### Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. For example, current chemotherapy for ovarian cancer often involves the combination of carboplatin (or cisplatin) and a taxane, such as paclitaxel (Taxol®) or docetaxel (Taxotere®) also added are bevacizumab (Alymsys®, Avastin®, Mvasi®, Zirabev®) and a poly ADP ribose polymerase (PARP) Inhibitor, such as, but, not limited to, niraparib (Zejula®) rucaparib (Rubraca®) and olaparib (Lynparza®). Additional examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolinodoxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifiuridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, famesyl-protein transferase inhibitors, transplatinum, and pharmaceutically acceptable salts, acids, or derivatives of any of the above.

### Radiotherapy

Other factors that cause DNA damage and have been used extensively include what are commonly known as gamma-rays, X-rays, and/or the directed delivery of radioisotopes to tumor cells. Other forms of DNA damaging factors are also contemplated, such as microwaves, proton beam irradiation (see, for example, U.S. Pat. Nos. 5,760,395 and 4,870,287), and U-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (mAbs) that are covalently linked to cell-killing drugs This approach combines the high specificity of mAbs against their antigen targets with highly potent cytotoxic drugs, resulting in “armed” mAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker payload optimization are becoming more and more mature, the discovery and development of new ADCs are increasingly dependent on the identification and validation of new targets that are suitable to this approach and the generation of targeting mAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and pi 55. An alternative aspect of immunotherapy is to combine anticancer effects with immune stimulatory effects. Immune stimulating molecules also exist including cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169); cytokine therapy, e.g., interferons, IL-1, GM-CSF, and TNF); gene therapy, e.g., TNF, IL-1, IL-2, and p53 (see, for example U.S. Pat. Nos. 5,830,880 and 5,846,945); and monoclonal antibodies, e.g., anti-CD20, anti-ganglioside GM2, and anti-p185 (see, for example, U.S. Pat. No. 5,824,311). It is contemplated that one or more anti-cancer therapies may be employed with the antibody therapies described herein.

### Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### Kits and Diagnostics

In various aspects of the embodiments, a kit is envisioned containing therapeutic agents and/or other therapeutic and delivery agents. In some embodiments, the present embodiments contemplate a kit for preparing and/or administering a therapy of the embodiments. The kit may comprise one or more sealed vials containing any of the pharmaceutical compositions of the present embodiments. The kit may include, for example, at least one anti-OSMR antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example NMR).

The term “binding affinity” or “K_{D}” refers to the dissociation rate of a particular antigen-antibody interaction. The Kp is the ratio of the rate of dissociation, also called the “off-rate (k_{off})”, to the association rate, or “on-rate (kₒₙ)”. Thus, Kp equals k_{off}/kₒₙ and is expressed as a molar concentration (M). It follows that the smaller the K_{D}, the stronger the affinity of binding. Therefore, a Kp of 1 M indicates weak binding affinity compared to a Kp of 1 nM. K_{D} values for antibodies can be determined using methods well established in the art. One method for determining the Kp of an antibody is by using surface plasmon resonance (SPR), typically using a biosensor system such as a Biacore® system.

The term “potency” is a measurement of biological activity and may be designated as IC50, or effective concentration of an antibody or antibody drug conjugate to the antigen OSMR to inhibit 50% of activity measured in a OSMR activity assay as described herein.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of an antibody molecule of the invention means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to OSMR.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as defined above. The term “treating” also includes adjuvant and neoadjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

The practice of the present invention will employ, unless otherwise indicated, conventional techniques of molecular biology (including recombinant techniques), microbiology, cell biology, biochemistry and immunology, which are within the skill of the art.

The present invention is illustrated by the following examples. It is to be understood that the particular examples, materials, amounts, and procedures are to be interpreted broadly in accordance with the scope and spirit of the invention as set forth herein.

### EXAMPLES

The following examples are included to demonstrate certain embodiments of the invention.

### Experimental Procedures and Methods

Unless noted otherwise, data generated from the experiments and Examples described hereinbelow can be found in (“Oncostatin M Receptor-Targeted Antibodies SupressSTAT3 Signaling and Inhibit Ovarian Cancer Growth”, Geethadevi et al., 2021, Cancer Res., 81:5336-52; which is incorporated herein by reference in its entirety.)

Patients and tissue samples: Ovarian cancer tissue and Normal ovarian tissue samples were obtained from Cancer Center and Department of Obstetrics & Gynecology, Froedtert Hospital, Medical College of Wisconsin. All human samples were collected with informed consents from patients after approval by the Institutional Review Board (IRB) of Medical College of Wisconsin.

TABLE 1
Patient characteristics of the TMA cohort.

| TMA data set Characteristics | Summary |
|---|---|
| Ovarian carcinoma patients | N = 110 |
| Age | 48.27 ± 1.31 years |
| Gender | Female |
| Number of tumor histopathology image | N = 110 |
| series | |
| Number of histopathology image cores | N = 150 |
| Number of NAT cores | N = 20 |
| Number of Normal Ovarian tissue cores | N = 10 |
| Number of Metastatic tissue cores | N = 10 (8%) |
| Number of Borderline tissue cores | N = 7 (5.6%) |
| Number of Benign tissue cores | N = 18 (14.4%) |
| Grade | |
| Grade 1 | N = 6 (5.45%) |
| Grade 2 | N = 23 (21%) |
| Grade 1-2 | N = 3 (2.72%) |
| Grade 3 | N = 20 (18.18%) |
| Grade 4 | N = 0 |
| Grade unavailable | N = 58 (52.72%) |
| High Grade serous Ovarian carcinoma | N = 23 (20.9%) |
| Low Grade serous Ovarian carcinoma | N = 4 (3.64%) |
| Stage | |
| Stage I | N = 29 (26.36%) |
| Stage II | N = 26 (23.64%) |
| Stage III | N = 24 (21.82%) |

Cell lines: Human ovarian cancer cells, HEYA8 was purchased from Characterized Cell Line repository at MD Anderson Cancer Center, Houston, TX, USA. OVCAR4, OVCAR5, OVCAR8, and CaOV3 were purchased from National Cancer Institute (NCI). NIH-OVCAR3 and SKOV3 were purchased from ATCC/PBCF repository. THP1 cell line was purchased from ATCC. MCAS cell line was received from Gordon Mills at MD Anderson Cancer Center, Houston, Texas, USA. FTE187 and FTE188 were kindly provided by Dr. Jinsong Liu (MD Anderson Cancer Center, Houston, TX). PEO1, and PEO4 were kindly provided by Daniela E Matei, Northwestern University, Chicago, Illinois, USA. HEK293-FT cell line was procured from Thermo Fisher Scientific. Untransformed fibroblast cell line (GM15859) was procured from Cornell Institute for Medical Research, NJ, USA. RF24 was received from Dr. Arjan W. Griffioen, VU University Medical Center, Amsterdam, Netherlands. Ovarian surface epithelial cells (OSE) were cultured by scraping the surface epithelium of normal ovarian tissues obtained from two patients with benign gynecologic pathology. All cell lines were cultured in Dulbecco's modified Eagle's medium (DMEM) (Sigma Aldrich). FTE187 and FTE188 were cultured in 1:1 MCDB 105 and Medium 199 (Thermo Scientific). NIH OVCAR3 was cultured in RPMI-1640 Medium with HEPES (ATCC). IOSE cells were cultured in Medium 199/MCDB105 (1:1, Sigma) supplemented with 15% FBS, 1% pen-strep, 10 ng/ml human epidermal growth factor (Peprotech, NJ), 0.5 g/mL hydrocortisone (Sigma), 5 g/mL bovine insulin (Sigma), 34 g protein/mL bovine pituitary extract (Life Technologies) (Barger et al., 2015). OSE cells were cultured in MEBM media (Lonza, Basel, Switzerland) supplemented with 15% FBS. Fibroblast cells were cultured in Eagle's Minimum Essential Medium with Earle's salts and non-essential amino acids and 10% inactivated FBS. Macrophage was cultured in RPMI-1640 Medium, with HEPES and 2-mercaptoethanol (0.05 mM). All cell lines were cultured in 10% fetal bovine serum (Atlanta Biologicals, GA, USA), or otherwise mentioned and supplemented with 1% Pen-strep (Thermo Fisher Scientific Inc., Waltham, MA, USA) at 37° C. in a humidified incubator with 5% CO2 as described previously (Parashar and Geethadevi et al 2019). The cell lines were authenticated by short tandem repeat (STR) profiling (IDEXX BioAnalytics) and tested for Mycoplasma using MycoSensor PCR Assay kit (Agilent, Santa Clara, CA).

Single cell/nucleus RNA-seq analysis: A total of 17 human ovarian cancer samples from single cell/nucleus RNA-seq data were analyzed in this study. The datasets OvD1-10×, OvD2-10× and OvD3-10×-nuc were single-patient ovarian cancer samples. The first two datasets were 10× droplet-based single cell RNA-seq (scRNA-seq) and the third was 10× droplet-based single nucleus RNA-seq (snRNA-seq) data. These were obtained from (Izar et al., 2020) with GEO accession no. GSE140819 (GSM4186985, GSM4186986 and GSM4186987, respectively). The raw counts matrix in h5 format and their corresponding cell annotations in metadata files were used for the analysis. The datasets OvD4-10×-mult and OvD5-SS2 were multi-patient ovarian cancer samples with n=6 (but 8 samples with multiple temporal sampling) and n=9, respectively. But one patient was common in OvD4-10×-mult and OvD5-SS2 dataset. The first dataset was 10× droplet-based scRNA-seq and the second was plate-based high-depth SMART-seq2 (SS2) scRNA-seq. The log-tpm-normalized data and the annotation were obtained from (Slyper et al., 2020) with GEO accession no. GSE146026.

The datasets were analyzed using Seurat v2.4 (Butler et al., 2018; Stuart et al., 2019). For OvD1, OvD2, and OvD3 datasets, only the cells present in the cell annotation metadata files and the genes present in at least 3 cells were considered. Data were normalized with ‘LogNormalize’ and scale factor 14. ‘ScaleData’ was performed by regressing UMI counts and percent of mitochondrial genes detected. OvD4 and OvD5 were log-tpm-normalized datasets and no further normalization were performed. ‘ScaleData’ was performed with default parameters. Rest of the steps were similar for all the datasets. ‘FindVariableGenes’ was performed to detect ˜2000 highly variable genes. ‘RunPCA’ step used default parameters and ‘PCElbowPlot’ was used to detect the significant number of PCA dimensions for ‘FindClusters’ and ‘RunUMAP’. Cell-type specific markers were identified from the literature and ‘FindAllMarkers’ command with ‘wilcox’ test and other default parameters. Visualization methods available in Seurat and custom codes in ggplot2 R package were used to generate the figures.

Ligand-receptor interaction analysis: CellPhoneDB (Efremova et al., 2020) is a curated repository of interactions between ligands and receptors along with the molecular subunit architecture information. These details are integrated in a statistical framework to infer cell-cell communication network in single-cell transcriptomics data. CellPhoneDB v.2.0.0 was used and the recommended procedures for preparation of input files (Efremova et al., 2020) were followed. Briefly, the log-normalized gene expression data and the metadata of cell type identities (obtained previously with clustering and cell-type specific markers) were used as the input. Ligand-receptor interactions were then identified using ‘cellphonedb method statistical_analysis’ command with default parameters. The interactions were visualized using ggplot2 R package.

Antibodies: OSMRβ antibody (Santa Cruz Biotechnologies, #sc-271695) for Western blot (1:700 dilution). OSMR antibody (Proteintech, #10982-1-AP) for IHC (1:50 dilution). OSM antibody (Proteintech, #27792-1-AP) for IHC (1:50 dilution). STAT3 (phospho Y705) antibody (Cell Signaling, #9145) for Western blotting (1:1000 dilution). STAT3 (phospho S727) antibody (Cell Signaling Technology, #9134) for Western blotting (1:1000 dilution). STAT3 antibody (Cell Signaling Technology, #9139) for Western blotting (1:1,000 dilution). LIFR antibody (R&D Systems, #AF-249-NA) for Western blotting (1:5000 dilution). IL31 antibody (Invitrogen, Thermo Scientific, #PA5-47391) for Western blotting (1:5000 dilution). IL6ST/gp130 antibody (Santa Cruz Biotechnologies, #sc-376280) for Western blotting (1:500 dilution). PCNA antibody (Santa Cruz Biotechnologies, #sc-25280) for Western blotting (1:1,000 dilution). BCLxL antibody (Cell Signaling Technology, #2764) for Western blotting (1:1,000 dilution). BCL2 antibody (Cell Signaling Technology, #15071) for Western blotting (1:1,000 dilution). Cytochrome c antibody (Cell Signaling Technology, #11940) for Western blotting (1:1,000 dilution). P27 antibody (Cell Signaling Technology, #3686) for Western blotting (1:1,000 dilution). β-Actin antibody (Santa Cruz Biotechnologies, #sc-8432) for Western blotting (1:1,000 dilution).

siRNA, lentiviral packaging and HEYA8 cells transduction: Two non-overlapping siRNA that targets coding region of human OSMR, one siRNA each of IL6R, CNTFR, IL27RA, and IL11RA and a non-specific siControl were purchased from Sigma Aldrich (Lafayette, CO) and siRNA of LIFR, IL6ST and IL31RA were purchased from Ambion (Life Technologies). SiRNAs were introduced into cells using RNAiMAX (Invitrogen, Carlsbad, CA) according to manufacturer's guidelines. The sequence of siRNAs is shown in Table 2.

TABLE 2
siRNA Sequences.

| Gene | Sequence |
|---|---|
| OSMR | siRNA#1: 5′-GUA ACU GCA UUC AAC UUG A-3′ |
| | (SEQ ID NO: 235) |
| OSMR | siRNA#2: 5′-CCA GAU CAG UAG GAU UGA A-3′ |
| | (SEQ ID NO: 236) |
| IL6ST | 5′-CCU CAU GCA CUG UUG AUU A-3′ |
| | (SEQ ID NO: 237) |
| LIFR | 5′-CCA CCU UCC AAA AUA GCG A-3′ |
| | (SEQ ID NO: 238) |
| IL31RA | 5′-CAA UGA ACA CUA ACC A-3′ |
| | (SEQ ID NO: 239) |
| IL6R | 5′-CCA GUA GUG UCG GGA GCA A-3′ |
| | (SEQ ID NO: 240) |
| CNTFR | 5′-GCC ACA AUG CCA CAG CUA U-3′ |
| | (SEQ ID NO: 241) |
| IL27RA | 5′-GAG UUG GAC CCU UGG GCG A-3′ |
| | (SEQ ID NO: 242) |
| IL11RA | 5′-CUG AGU UCU GGA GCC AGU A-3′ |
| | (SEQ ID NO: 243) |

For establishment of stable cell lines, two non-overlapping pLKO.1-shOSMR expression plasmids (MISSION® pLKO.1-puro-shOSMR #1 TRCN0000058687, Clone ID: NM_003999.1-2459slc1, 5′CCGGGCATTGATTGTGGACAACCTACTCGAGTAGGTTGTCCACAATCAATGCTT TTTG3′ (SEQ ID NO: 244) and MISSION® pLKO.1-puro-shOSMR #2: TRCN0000289933 Clone ID: NM_003999.1-2857s21c1, 5′CCGGCGAGTTGACTAAGCCTAACTACTCGAGTAGTTAGGCTTAGTCAACTCGTT TTTG-3′) (SEQ ID NO: 245) targeting the coding region of human OSMR and human MISSION® pLKO.1-puro Empty Vector control plasmid (Cat #SHC001V) were purchased from Sigma Aldrich (Saint Louis, MO). To produce lentiviral particles, pLKO.1-shOSMR expression plasmids and pLKO.1-control plasmid were co-transfected with three lentivirus packaging plasmids pLP1, pLP2, pLP/VSVG (Addgene) into HEK293-FT cell line Thermo Scientific, Inc.) using Lipofectamine 2000. Competent lentiviruses were collected 48 h after transfection. To generate stable OSMR knockdown cells, the tittered lentiviral particles containing shOSMR were transduced in HEYA8-Luc+ cells passaged to 60-70% confluency and Puromycin (final concentration=8 g/mL) was used to select infected cells along with polybrene (8 μg/mL). Transduction efficiency of each shOSMR was checked by Western Blot using OSMR antibody. The most effective shRNA construct was used for generating OSMR knockdown stable cell lines by selection with puromycin (8 g/ml; for over 2 weeks). For consistency of results, the effective shHeya8-Luc+ cell lines were subjected to selection with Puromycin every two months.

OSMR overexpression plasmid preparation: For establishment of stable overexpression cell lines, pUNO1-OSMR (#puno1-hosmr) vector targeting the coding region of human OSMR and non-specific control pUNO1-Control (#puno1-mcs) vector were purchased from InvivoGen (San Diego, CA, USA). The overexpression pUNO1-OSMR and pUNO1-Control plasmids were transfected in HEYA8-Luc+ and OVCAR5 cell lines using Lipofectamine 2000 and Blasticidin (final concentration=10 μg/mL) was used to select transfected clones. Selected OSMR overexpression clones were subjected to Western blot analysis to check the efficiency.

### Example 1—Production, Screening and Purification of Specific Antibodies

Phage library panning for antibodies to the OSMR: The OSMR protein (Sino Biologicals, 11226-H08H) was used for antibody selection by panning a large human scFv phage display antibody library. The library was constructed in house from the cDNA extracted from the PBMCs and tonsils of multiple donors. In each round of phage panning, 50 μg of protein was coated on a MaxiSorp immune tube and blocked by 8% milk. The phages were pre-blocked by 8% milk then incubated with the antigen pre-coated on the immune tube. After washing with PBST and PBS, the phages were eluted by triethylamine (TEA). The eluates were tittered and infected E. coli TG1 for phage amplification for next round of panning. Similar procedures were performed in round 2 of panning with increased washing stringency. After 2 rounds of panning, the phage eluates were used to infect E. coli TG1 to grow single colonies for picking by QPix420 system (Molecule Devices) and for phage preparation.

Phage ELISA: Screening of specific antibody leads was performed by sandwich ELISA. A total of 1504 single colonies were picked to make phage for ELISA binding with OSMR. ELISA plates were coated with OSMR antigen at 1 μg/mL in PBS for overnight at 4° C. The plates were blocked with 5% milk for 2 hours at 37° C. and the phages were pre-blocked with 5% milk for 1 hour at room temperature. After blocking, the phages were added into the wells of the ELISA plates and incubated for 2 hours at 37° C. An HRP-conjugated Mouse-anti-M13 secondary antibody was 1:1000 diluted in 5% milk and added into the wells for incubation for 1 hour at 37° C. The plates were washed 3 times between each incubation steps and 5 times before color development. The TMB substrate was added into the wells (100 μl/well) for color development for 5 mins. The H₂SO₄ was used to stop the reaction.

IgG expression and purification: A total of 500 phage clones which were positive in the phage ELISA were sequenced for the scFv regions. After analysis of the complementarity-determining regions (CDRs), 35 scFvs with unique amino acid sequences were obtained and further subjected to conversion into full IgG1 heavy chain and light chain constructs. These constructs were co-transfected Expi293 cells for expression of recombinant antibodies according to manufacturer's instructions. After 7 days, antibodies were purified by affinity chromatography using protein A resin. A total of 26 antibodies were produced for further experiments.

Affinity measurement with BLI: For antibody affinity measurement, antibody (20 μg/mL) was loaded onto the protein G biosensors for 150 s. Following a short baseline in kinetics buffer, the loaded biosensors were exposed to a series of recombinant OSMR concentrations (0.41-900 nM) and background subtraction was used to correct for sensor drifting. All experiments were performed with shaking at 1,000 rpm. Background wavelength shifts were measured from reference biosensors that were loaded only with antibody. ForteBio's data analysis software was used to fit the data to a 1:1 binding model to extract an association rate and dissociation rate. The Kd was calculated using the ratio k_{off}/kₒₙ.

### Example 2—Treatment of Cancer with Monoclonal Antibodies

Animal studies: All animal work was done in accordance with protocol approved by the Institutional Animal Care and Use Committee (IACUC) at the Medical College of Wisconsin. To compare the ability of OSM, LIF and IL31 on ovarian cancer growth, HEYA8-Luc+ cells were treated with Vehicle/PBS, OSM (100 ng/mL), LIF (100 ng/ml) and IL31 (50 ng/mL) in vitro for 24 h and subcutaneously (s.c) injected (1×10⁵ cells/animal) into 4-6 weeks old athymic female nude mice (Nu/Nu) (Envigo, Madison, WI, USA) per flank. The mice were treated intraperitoneally (i.p) with Vehicle/PBS, OSM (250 ng/kg b.w), LIF (250 ng/kg b.w) and IL31 (250 ng/kg b.w) twice a week for total of 5 weeks. Palpable tumors were measured using Vernier caliper once every week. Tumors were excised at the endpoint, weighed and proceeded for Western blotting analysis. Tumor volume was calculated by the formula V=(W (2)× L)/2.

To study the effect of stable knockdown of OSMR on the ovarian cancer tumor progression, shOSMR #1-Heya8-Luc+ cells (3×10⁴ cells/animal) and shControl-HEYA8-Luc+ cells were intraperitoneally injected into 4-6-week-old athymic female nude mice (Nu/Nu) (Envigo, Madison, WI, USA) (N=7/group) with a 27-gauge needle. Mice were monitored for tumor growth once every week by bio-luminescence imaging using Xenogen IVIS100 imaging system (Caliper Life Sciences Inc, Waltham, MA). All mice were euthanized at the end of 5 weeks or when moribund. Tumors were harvested and weighed, total number of tumor nodules and metastasis to distant organs were counted and imaged using IVIS100. The tumors were excised and then proceeded for IHC, Western blotting and qPCR. Serum was collected from both groups for OSM ELISA.

To test the in vivo efficacy of anti-OSMR antibodies and overall survival efficiency post-treatment, Heya8-Luc+ cells (3×10⁴ cells/animal) were intraperitoneally injected into 4-6-week-old athymic female nude mice (Nu/Nu) (Envigo, Madison, WI, USA) with a 27-gauge needle. Mice were monitored for tumor growth by bio-luminescence imaging IVIS100 and after 7 days post injection, randomly divided into 7 mice per group (efficacy testing) and 10 mice per group (survival studies). Mice were treated intraperitoneally twice weekly with Control IgG.

B14 mAb, or B21 mAbs, with or without OSM (250 ng/kg b.w) twice a week for five weeks. All the antibody clones were given at a concentration 10 mg/kg b.w twice a week for 5 weeks. All mice were euthanized at the end of 5 weeks or when moribund. Tumors were harvested and weighed, total number of tumor nodules and metastasis to distant organs were counted and imaged using IVIS100. The tumors were excised and proceeded for IHC, Western blotting and qPCR. For survival studies, the treatments were stopped after 5 weeks, and survival progression was monitored for a total of 100 days.

Migration and invasion assay: The effect of stable OSMR overexpression or knockdown was analyzed by carrying out cell migration and invasion assay as described earlier (Jagadish et al., 2015) HEYA8 cells (1×10⁵) suspended in serum-free medium were seeded into 1 mg/mL Matrigel-coated (invasion) or uncoated (migration) cell culture inserts (8-μm; Millipore, Billerica, MA). Medium with 10% FBS was added to the lower chambers. Migration and invasion assays were performed in the presence of cell cycle inhibitors mitomycin C (5 μg/ml), in the trans-well chambers for 12 h or 16 h respectively. Cells from the upper chamber were removed using a cotton swab. Invaded or migrated cells were fixed by 5% glutataldehyde and stained in 0.5% crystal violet in 20% methanol and photographed. Membranes were then removed and dissolved in 10% acetic acid and quantified in microplate reader at 560 nm.

3-Dimensional (3-D) culture of tumor cells: Tumor cells were cultured as 3-D spheroids as described previously (Parashar et al., 2019). Cells were harvested, counted and seeded onto Ultra Low Attachment 24-well Culture plates (Corning Life Science, Catalog number 3261) and suspended in 1:1 ClonaCell medium: DMEM. The seeded cells and formed spheres were cultured in complete medium up to two weeks based on the requirement. Unless specified, media was replenished every two days.

Colony formation assay: Colony formation assay was performed as mentioned previously (Jagadish et al., 2016) with some modifications. Briefly, the cells were seeded into six-well plates at 400 cells per well with complete growth medium 24 h prior to treatment and cultured for 10 days. On the tenth day, the colonies were fixed with 5% gluteraldehyde and stained with 0.5% crystal violet in 20% methanol. Plates containing the colonies were washed with water and dried before imaging. Colonies were solubilized with 10% acetic acid and quantified by reading absorbance at 560 nm.

CCK8 cell viability assay: One thousand cells were seeded each well in 96-well plates. Following treatment for 48 h, the cells were washed with PBS and incubated for 4 h with CCK8 reagent at 37° C. in 5% CO2 incubator according to manufacturer's guidelines. The absorbance was measured at 460 nm.

Wound-healing assays: Wound-healing assay was performed as mentioned previously (Kanojia et al., 2013) with some modifications. Briefly, cells were seeded on 6-well plates and grown to confluency following the respective treatments. Subsequently, the monolayer was gently scratched with a pipette tip to create a mechanical wound. Phase-contrast images of three to five selected fields were acquired for at least 32 h. Images were analyzed using Image J software.

Cell viability assay and Receptor internalization using Live Cell Incucyte Analyzer: The IncuCyte® Live Cell imaging system (Essen BioScience, Ann Arbor, MI) Romano et al, 2018) was used for assessment of proliferation. Briefly, 3×10³ viable HEYA8 cells were seeded in four replicates in a 96-well plate (TPP) containing complete medium. Following serum starvation, the cells were treated with Control IgG (10 μg/mL) or OSM (100 ng/ml) stimulation with or without anti-OSMR antibodies (10 μg/mL) and incubated with 1:200 dilution of IncuCyte® Annexin V Red Reagent for apoptosis (Essen Bioscience) for a period of 48 h. Cell viability was measured in real-time using the IncuCyte by taking 3 field images per well every 6 h. Masking was done using the IncuCyte® S3 Software. Red cell counts based on number of red apoptotic cells were done using the IncuCyte® Base Software Analysis Interface module (Essen Bioscience).

For receptor internalization, anti-OSMR antibodies B14 and B21 (10 μg/mL each) were labeled with pH sensitive FabFluor (Red) reagent (Essen Bioscience) as per manufacturer's instructions and added to serum starved ovarian cancer cells in the presence of OSM (100 ng/mL). The cells were also treated with Isotype Control IgG (10 μg/mL) in the presence and absence of recombinant OSM (100 ng/mL). The cells were then monitored in IncuCyte Live Cell Analysis system for 24 h. The antibody labeled with FabFluor Red reagent is non-fluorescent at neutral pH (outside cell). In due course, if the antibody-receptor complex gets internalized, due to acidic pH of endosomal and lysosomal compartments, antibody labeled with pH sensitive dye fluoresce red in the form of clusters inside cytoplasm which is then analyzed using the IncuCyte® S3 Software.

Western blot analysis and protein array: Cells were washed twice in ice-cold PBS and lysed in RIPA buffer (150 mM NaCl, 0.1% SDS, 1% NP-40, 1% sodium deoxycholate) (Santa Cruz Biotechnologies) supplemented with protease inhibitor cocktail (Sigma Aldrich) as mentioned previously (Parashar et al.). Total protein concentrations were determined using BCA kit (Thermo Fisher Scientific) and 30 μg protein was separated by pre-cast 4-12% SDS-PAGE and transferred onto PVDF membranes (Bio-Rad, Hercules, CA). After blocking by 5% non-fat milk (Bio-Rad, Hercules, CA), membranes were incubated with indicated primary antibodies overnight at 4° C. and corresponding HRP-conjugated secondary antibodies (Cell Signaling Technology). Immunoreactive signals were detected using chemiluminescence detection kit (Bio-Rad, Hercules, CA).

A Proteome Profiler Human Apoptosis Array Kit (Cat #ARY009; R&D Systems) was used to analyze apoptosis-related protein profiles and Human Phospho-Kinase Antibody Array kit (Catalog #ARY003B; R&D Systems) was used to analyze proteins phosphorylated by OSM or inhibition of phosphorylation by anti-OSMR antibodies according to manufacturer instructions. In brief, the total protein isolated from OVCAR4 cells after treatment with Control IgG, OSM (10 g/mL) and anti-OSMR antibodies B14 and B21 (10 μg/mL each) in the presence of OSM for 24 h (Protein kinase array) and for 48 h (Apoptosis array) were first incubated with the array membrane overnight at 4° C., followed by incubation with a biotinylated detection antibody cocktail at room temperature for 1 h. The membranes were then exposed to X-Ray film and quantified by Image J software (National Institutes of Health, Bethesda, USA).

Dimerization assay: Dimerization assay was performed as mentioned previously (Bublil et al., 2010; Turk and Chapkin, 2015) with some modifications. HEYA8 and OVCAR4 cells were seeded overnight and upon 70-80% confluency, serum starved overnight and pre-treated with Control IgG, B14 and B21 mAbs for 4 h then stimulated in the presence of OSM (100 ng/mL) for 60 min on ice to prevent internalization of dimerized receptors. The cell lysates were incubated with non-permeable cross-linking reagent, 3 mM bis (sulphosuccinimidyl) suberate [BS3 (Pierce)], cross-linking reagent on ice for 30 min and subsequently quenched with 250 mM Glycine. The cells were washed with ice cold PBS and cells were lysed using RIPA buffer as mentioned earlier. The pre-cleared lysates were immunoprecipitated overnight at 4° C. using anti-OSMR antibody bound to Dynabeads (Thermo Fisher Scientific) and eluted using 1× Laemmli sample buffer and the proteins were separated on a 6% SDS/PAGE. Separated proteins were transferred to PVDF membrane and immunoblotted with OSMR, IL6ST or IL31RA antibodies.

Receptor internalization: Receptor internalization was performed as mentioned previously (Phuchareon et al., 2015) with some modifications. Briefly, cells were seeded in 100 mm culture dish and once adhered, cells were cultured without serum for 16 h and treated with Control IgG and anti-OSMR antibodies (10 μg/mL each) along with OSM (100 ng/mL) for 6 h. Cells were washed with ice cold PBS and membrane and cytosolic protein fractions were isolated using Mem-PER™ Plus Membrane Protein Extraction Kit (Thermo Fisher Scientific) according to manufacturer's guidelines. Protein concentration in each fraction was determined using BCA kit and the 30 μg protein lysates from each fraction was separated by 8% SDS-PAGE.

On-Cell Western assay: Cells were seeded on 96 well (black plate) at a density of 5×10⁴ cells per well. Following serum starvation, cells were treated with B14 and B21 antibodies (10 μg/mL each) along with OSM (100 ng/mL). Cells were also treated with Isotype control IgG (10 μg/mL each) with and without OSM (100 ng/mL) for 16 h and washed with ice cold PBS and fixed with 4% paraformaldehyde. The cells were blocked for 1 h in LICOR blocking buffer and incubated with primary antibody against extracellular domain of OSMR (1:100 dilution, Cat: 11226-RP02 Sino Biologicals, Wayne, PA) along with Na+K+ ATPase (Santa Cruz Biotechnologies) overnight at 4° C. The cells were further washed with PBS and incubated with secondary antibodies IRDye® 680RD Donkey anti-Rabbit (Red) (1:800 dilution) and IRDye® 800CW Goat anti-Mouse IgG (Green) (1:200 dilution) (LI-COR, Lincoln, Nebraska) for 1 h and developed in Odyssey Scanner (LI-COR). The fluorescence intensity was quantitated using Li-Cor image studio software.

Cytokine ELISA: The levels of OSM in serum were determined by human OSM ELISA kit (R&D systems) according to manufacturer's guidelines and as previously mentioned (Parashar et al., 2019). Blood was collected from tumor-bearing mice and was allowed to clot for 30 min at room temperature before being centrifuged at 16,000×g for 10 min at 4° C. and the serum was aspirated. Briefly, an anti-human OSM antibody was pre-coated onto microwells. Human OSM present in the sample or standard binds to antibodies adsorbed to the microwells. Following incubation, unbound biological components are removed during a wash step. A biotin-conjugated anti-human OSM antibody is added and binds to human OSM captured by the first antibody. Following incubation unbound biotin-conjugated anti-human OSM antibody is removed during a wash step. Streptavidin HRP is added and binds to the biotin-conjugated anti-human OSM antibody. A colored product is formed in proportion to the amount of human OSM present in the sample or standard. The reaction is terminated by addition of acid and absorbance is measured at 450 nm.

Tissue microarray (TMA) and immunohistochemistry: Formalin-Fixed Paraffin-Embedded (FFPE) tissue array cores (OV1005bt and OV1004) consisting of 5 μm tissue sections from ovarian cancer patients, normal and normal adjacent ovarian tissue sections were procured from US Biomax Inc (Rockville, MD). For the expression of proteins in the tissues sections from tumor bearing mice, the tissues were fixed overnight in formalin jars and sections were paraffin embedded. Hematoxylin and eosin (H& E) staining was used to counterstain the tissues from all treatment groups.

TMA was performed as previously mentioned (Chen et al., 2020). Briefly, the sections were deparaffinized and rehydrated through graded alcohols. The sections were incubated in 3% H₂O₂ to reduce endogenous peroxidase activity and antigen retrieval was performed using Antigen retrieval buffer (IHC World, Woodstock, MD) for 60 min. The sections were blocked in goat serum. Incubation with primary antibody (1:60 dilution for OSMR & OSM, 1:100 for Ki67, pSTAT3 and Cleaved Caspase 3) was performed overnight at 4° C. followed by AP-conjugated secondary antibody (Vector Labs) for 1 h at room temperature. For staining Vectastain ABC-AP Kit (Vector Labs, Burlingame, CA) and Vector Red Alkaline Phosphatase Substrate Kit I (Vector Labs, Burlingame, CA) were used according to manufacture protocol. Protein expression was represented by IHC score (0-5), which was calculated for each section by adding the score of percentage of positive cells (intense red staining) (0≤5%, 1=6-20%, 2=21-40%, and 3=41-60%, 4=61-80%, 5=81-100%) and the intensity score: 0 (negative), 1 (weak), 2-3 (moderate), and 3.1-4 (high) and 4.1-5 (very high).

Real-time PCR and qPCR-based array analysis: Total RNA was extracted using TRIzol reagent (Life Technologies, Carlsbad, CA) and quantified on Nanodrop 2000 (Thermo Scientific). One microgram of total RNA was reverse transcribed using iScript Reverse Transcription Supermix for RT-qPCR (Bio-Rad). Real-time PCR was performed with iTaq Universal SYBR Green Supermix (Bio-Rad) according to manufacturer's instructions using a Bio-Rad CFX Connect Real Time PCR system (Bio-Rad). The abundance of mRNA was determined using the ΔΔCT method (where Cq is threshold cycle). mRNA expression was normalized to β-Actin (ACTB) mRNA. Sequences of the primers used are shown in Table 3.

TABLE 3
List of qPCR primers

| Genes | Direction | Sequence (5′→3′) |
|---|---|---|
| OSMR | Forward | CGC AGC AAG ACG GGA AAT TG |
| | | (SEQ ID NO: 246) |
| OSMR | Reverse | GGG CGG GAG TTA CTG GAA AA |
| | | (SEQ ID NO: 247) |
| IL31RA | Forward | ACG TAC AAC CTC ACG GGG CT |
| | | (SEQ ID NO: 248) |
| IL31RA | Reverse | TGG CCT TCT TCC ATC CGC CT |
| | | (SEQ ID NO: 249) |
| IL6ST | Forward | TGT GAG TGG GAT GGT GGA AGG G |
| | | (SEQ ID NO: 250) |
| IL6ST | Reverse | AGT GCA TGA GGT GGG GGT GT |
| | | (SEQ ID NO: 251) |
| STAT3 | Forward | GGA GGA GTT GCA GCA AAA AG |
| | | (SEQ ID NO: 252) |
| STAT3 | Reverse | TGT GTT TGT GCC CAG AAT GT |
| | | (SEQ ID NO: 253) |
| IL6RA | Forward | CCA TGC AGG CAC TTA CTA CT |
| | | (SEQ ID NO: 254) |
| IL6RA | Reverse | GGC AGT GGT ACT GAA GAA GAA |
| | | (SEQ ID NO: 255) |
| IL11RA | Forward | CCG GAT TAA TGT GAC TGA GGT G |
| | | (SEQ ID NO: 256) |
| IL11RA | Reverse | GGG TAA CCT GGT ACT GAC TCT A |
| | | (SEQ ID NO: 257) |
| LIFR | Forward | CAT CAT CAG CGT AGT GGC TAA A |
| | | (SEQ ID NO: 258) |
| LIFR | Reverse | GTA ATG CCA GGT GAG GAG AAT C |
| | | (SEQ ID NO: 259) |
| CNTFR | Forward | CAG CAC ACA CCA TCA CAG A |
| | | (SEQ ID NO: 260) |
| CNTFR | Reverse | GTC ACT CCA TGT CCC AAT CTC |
| | | (SEQ ID NO: 261) |
| IL27RA | Forward | CTG TGG GTG ACA GCA TCT AC |
| | | (SEQ ID NO: 262) |
| IL27RA | Reverse | CTT TCC ACC TCA GGG TGT TAT C |
| | | (SEQ ID NO: 263) |
| ACTB | Forward | GTC ATT CCA AAT ATG AGA TGC GT |
| | | (SEQ ID NO: 264) |
| ACTB | Reverse | GCT ATC ACC TCC CCT GTG TG |
| | | (SEQ ID NO: 265) |

Gene set enrichment analysis: For Gene set enrichment analysis (GSEA), gene expression data of ovarian cancer was obtained from TCGA project. The expression of genes was measured by fragments per kilobase of exon model per million reads mapped (FPKM). After calculating the Pearson Correlation Coefficient (PCC) between the expression of genes and OSM or OSMR, all genes were ranked based on PCC and then subjected to GSEA analysis (Subramanian et al., 2005). Enrichment score (ES) was calculated for each functional set, which reflects the degree to which a gene set is overrepresented at the top or bottom of the ranked list of genes. The normalized enrichment score (NES) was calculated based on 1000 permutations. Here, the cancer hallmark gene sets from MSigDB were considered and the gene sets with false discovery rate <0.001 were considered as a selection criterion (Liberzon et al., 2015; Subramanian et al., 2005).

Statistical analysis: Cell culture-based experiments were repeated at least three times (three biological replicates) and all data were expressed as means±SE. Significance was assessed by unpaired two-tailed Student's t-test using GraphPad Prism. Comparison analysis between two treatment groups in animal models was performed by One-way ANOVA followed by Dunnett's multiple comparison test. Statistical analysis of the animals treated in the survival model was done by a log-rank (Mantel-Cox) test. The differences were considered to be statistically significant for P-values <0.05 (*), <0.01 (**), <0.001 (***), and <0.0001 (****).

It is now known that OSMR is differentially expressed in ovarian cancer cells and in cancer associated fibroblasts. To identify targetable IL6 family receptors that are differentially expressed in ovarian cancer cells and cancer associated cells, the inventors analyzed droplet-based three single-cell RNA sequencing (scRNA-seq) datasets (OvD1-10×, OvD2-10× and OvD4-10×-mult) and one single-nucleus RNA sequencing (snRNA-seq) dataset (OvD3-nuc) of human ovarian cancer patient samples (Izar et al., 2020; Slyper et al., 2020) and determined the expression of all IL6 family receptors such as IL6ST, OSMR, IL27RA, LIFR, IL11RA, IL6R, CNTFR, and IL31RA. There were 9 patients and 11 samples in these droplet-based datasets, where the inventors found OSMR and its dimerizing partner IL6ST among the top highly expressed receptors in ovarian cancer cells, cancer associated fibroblasts and endothelial cells compared to immune cells.

Although, droplet-based scRNA-seq and snRNA-seq have high sequencing coverage, however, both approaches exhibit some limitations because of high chances of dropout of genes due to low-depth coverage in sequencing (Ziegenhain et al., 2017). Therefore, the inventors used a plate-based, low dropout and high-depth SMART-seq2 (SS2) scRNA-seq data (OVD5_SS2) from n=9 human ovarian cancer patients and confirmed the results from 10× datasets. This analysis again confirmed that OSMR and IL6ST are the top highly expressed IL6 family receptors in cancer cells. The inventors also observed that OSMR is highly expressed in ovarian cancer cells and cancer associated fibroblasts, and in a lesser degree in macrophages. Notably, the inventors found that OSM, which is the ligand of OSMR, is mainly produced by macrophages.

Next, all the known ligand-receptor interactions between different cell types in OvD5-SS2 dataset were determined using CellPhoneDB (Efremova et al., 2020) and selected all the interactions involving IL6 family ligands and their receptors. Here, it was found that OSMR, IL6ST, LIFR, IL11RA and IL6R showed significant cell-cell interactions with their ligands expressed by macrophages and fibroblasts.

However, LIFR and IL11RA are expressed in very few ovarian cancer cells, whereas IL6R is ubiquitously expressed in all cell types and particularly high in immune cells. Notably, IL6ST is also expressed highly in all cell types, whereas its dimerizing partner OSMR is highly expressed predominantly in ovarian cancer cells, tumor associated endothelial cells and fibroblasts. Analysis further showed that IL6ST interacts with multiple IL6 family ligands such as OSM, IL6, and IL11, which leads to the dimerization of IL6ST with multiple IL6 family receptors such OSMR, LIFR, IL6R, and IL11RA, CNTFR, and IL27R (Rose-John, 2018). Surprisingly, the inventors discovered that OSMR interacts with only OSM and heterodimerizes with IL6ST. Though IL6ST is highly expressed in ovarian cancer cells, its ability to dimerize with multiple chemokine and cytokine receptors for vital functions of immune cells limits its potential as a highly specific cancer target. Thus, long felt need to characterize and focus on the functions of OSMR, which is the second most and highly expressed IL6 family receptor in ovarian cancer cells as a therapeutic avenue to treat ovarian cancer was addressed.

OSM-signaling through OSMR is a critical mechanism for pathological characteristics of ovarian cancer The protein levels of OSMR subfamily of receptors, which include IL6ST, LIFR, IL31RA and OSMR, were examined in a panel of ovarian cancer cell lines and found that OSMR is highly upregulated in most of the aggressive ovarian cancer cells as compared to fallopian tube epithelial cells such as FTE cell lines whereas IL6ST receptor is also highly expressed in most of the cell lines, however there is no significant changes in the expression between FTE and ovarian cancer cells. In conjunction with the single cell analysis results, the inventors found that LIFR and IL31RA are poorly expressed in ovarian cancer cell lines.

To further validate OSMR and IL6ST are critical for oncogenic signaling in ovarian cancer cells, the inventors determined the protein expression of all the OSMR subfamily receptors (OSMR, IL6ST, IL31RA and LIFR) and other IL6 family receptors, in ovarian cancer tissues and adjacent normal tissue. that the results indicated OSMR is highly and differentially expressed in cancer tissues compared to normal adjacent tissues (NAT); whereas it was noticed that IL6ST is highly expressed in all samples and not differentially expressed between NAT and ovarian cancer tissues. Similar to the single cell/nucleus RNA-seq datasets, the inventors found that LIFR is poorly expressed and there is no change in its level between NAT and cancer tissues. It was found IL31RA has little or no expression in both normal and ovarian cancer tissues. Next, the effect of secreted OSM on dimerization of OSMR with itself and with IL6ST, which is the first process for oncogenic signaling in ovarian cancer was determined and it was found that OSM stimulation improved the dimerization between OSMR-OSMR and OSMR-IL6ST in OVCAR4 and HEYA8 ovarian cancer cells.

Without being bound by theory, the inventors found that increased expression of OSMR in fibroblasts, macrophages and endothelial cells in ovarian cancers, suggests that the association or interaction of said fibroblasts, macrophages and endothelial cells with tumor cells as a potential reason to upregulate OSMR expression in fibroblasts, macrophages and endothelial cells. To this hypothesis, the inventors determined the mRNA expression of OSMR family receptors in fibroblasts, macrophages (THP1) and endothelial cells (RF24) that were either grown alone or co-cultured with normal ovarian epithelial cells (OSE) and ovarian cancer cells (HEYA8 and OVCAR4). Surprisingly, the inventors discovered the results indicated both OSMR and IL6ST were upregulated highly in endothelial cells and fibroblasts, and modestly in macrophages when co-cultured with ovarian cancer cells compared to the cells when cultured alone or co-cultured with OSE cells; whereas the inventors observed low to modest change in the expression of IL31RA expression in fibroblasts, macrophages and endothelial cells when co-cultured with ovarian cancer cells. It was observed that LIFR and IL6R were upregulated highly in endothelial cells and macrophages respectively, when co-cultured with cancer cells. Taken together, the results suggest that the increased expression of OSMR and IL6ST expression in fibroblast, endothelial cells is potentially due to their association with cancer cells.

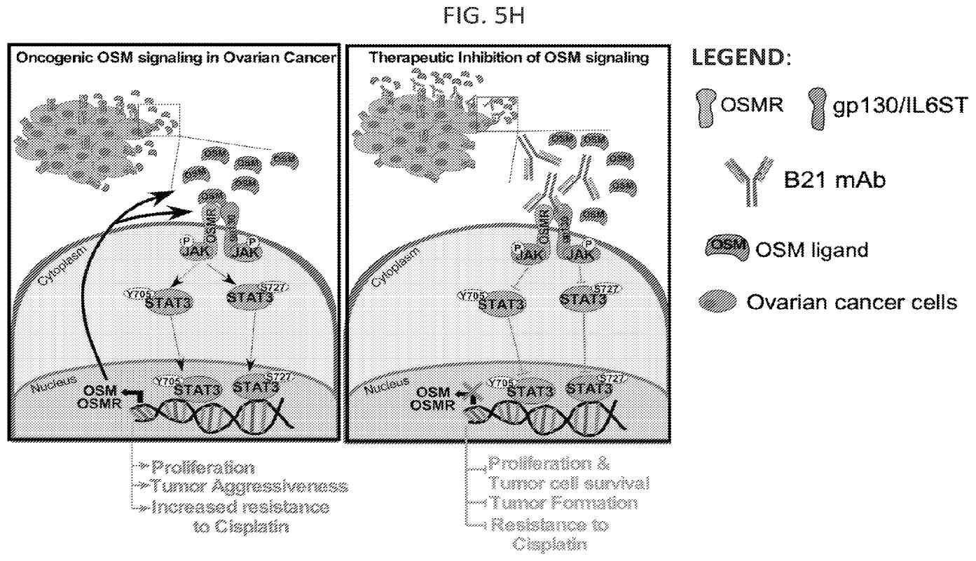

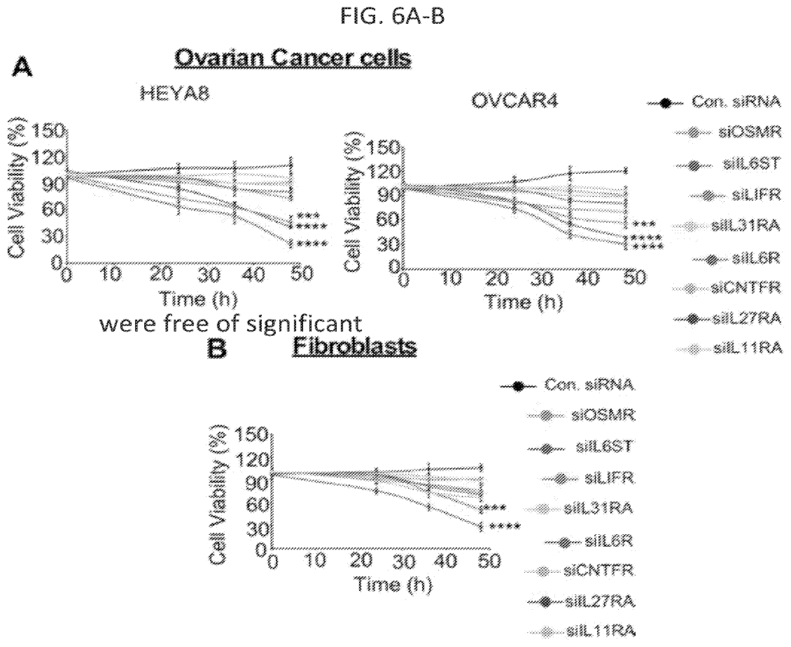

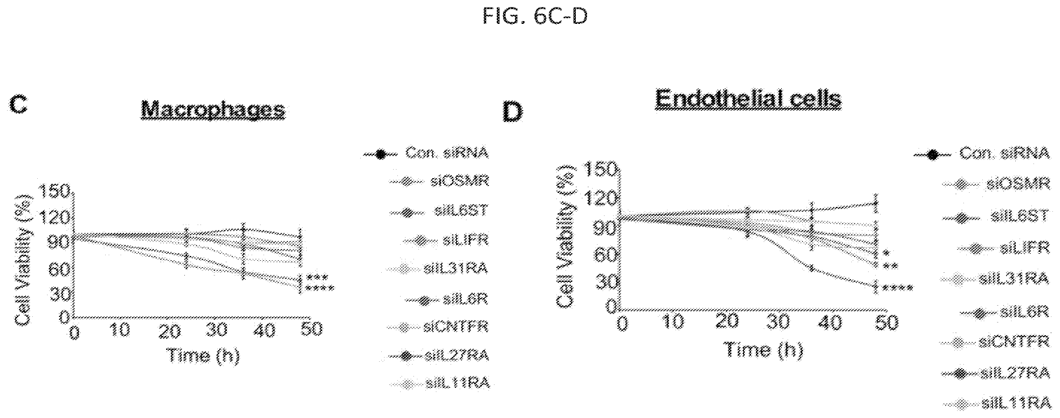

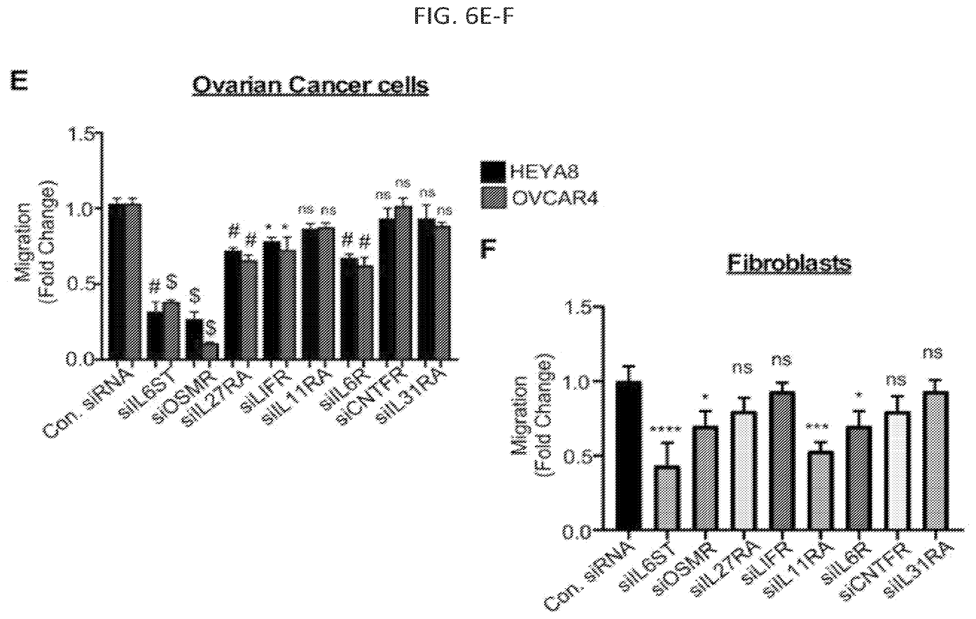

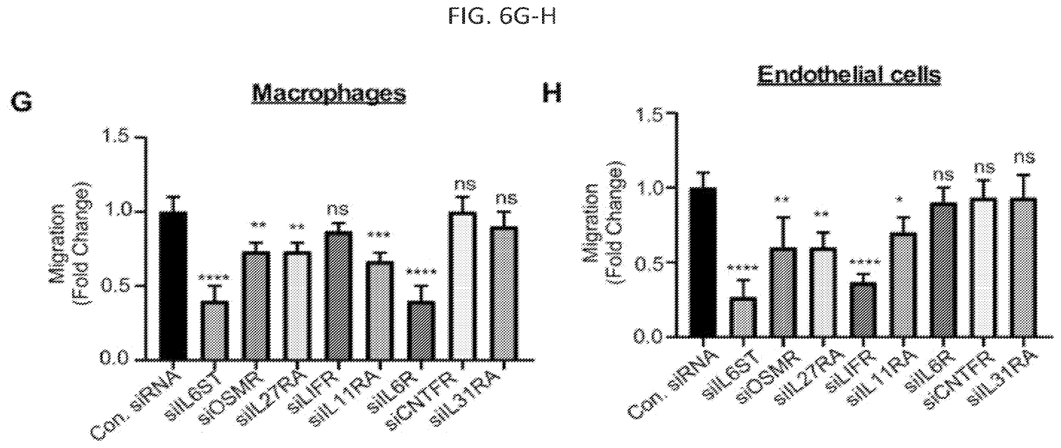

To further confirm the importance of OSM family receptors on oncogenic characteristics in both ovarian cancer cells and the cells in TME, the inventors knocked down all the IL6 subfamily genes in ovarian cancer cells, and cells in TME such as fibroblast, endothelial (RF24) and macrophage (THP1) cells and determined cell proliferation and migration. that the results indicated the loss of OSMR considerably reduced the proliferation (FIG. 6a) and migration (FIG. 6e) in ovarian cancer cells (>70%) but did not exert any major effects on the proliferation and migration of non-cancer cells (FIGS. 68b to 6d and FIGS. 6e to 6h). It was further observed that the knockdown of IL6ST and IL6R reduced the proliferation and migration of ovarian cancer cells low to modest levels compared to the knockdown of OSMR (FIGS. 6a, 6b, and 6e), whereas it was also noticed that the loss of IL6ST and IL6R inhibited the proliferation and migration of fibroblast, endothelial and macrophage lines significantly (FIGS. 6b to 6d and FIGS. 6e to 6h). Taken together the data demonstrate that OSMR is an important regulator of ovarian cancer cell proliferation and migration precisely compared to other IL6 family receptors.

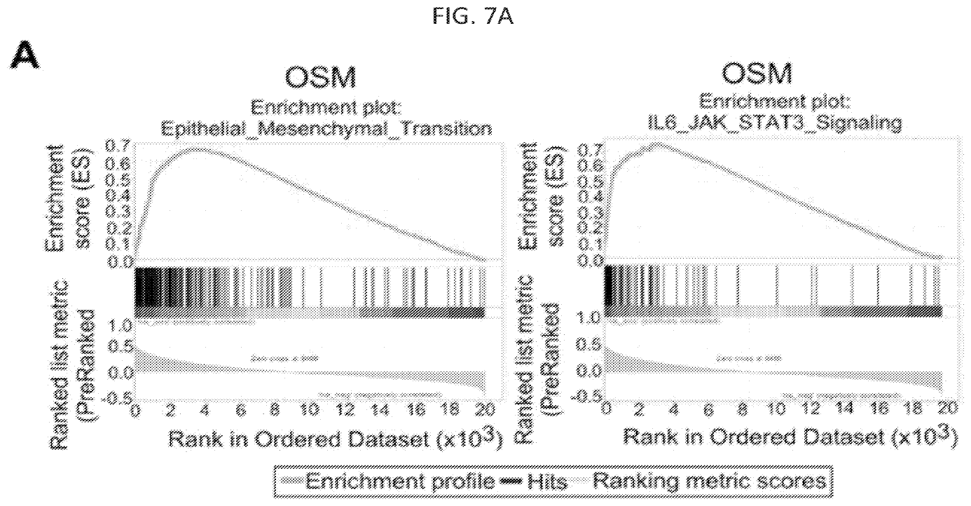

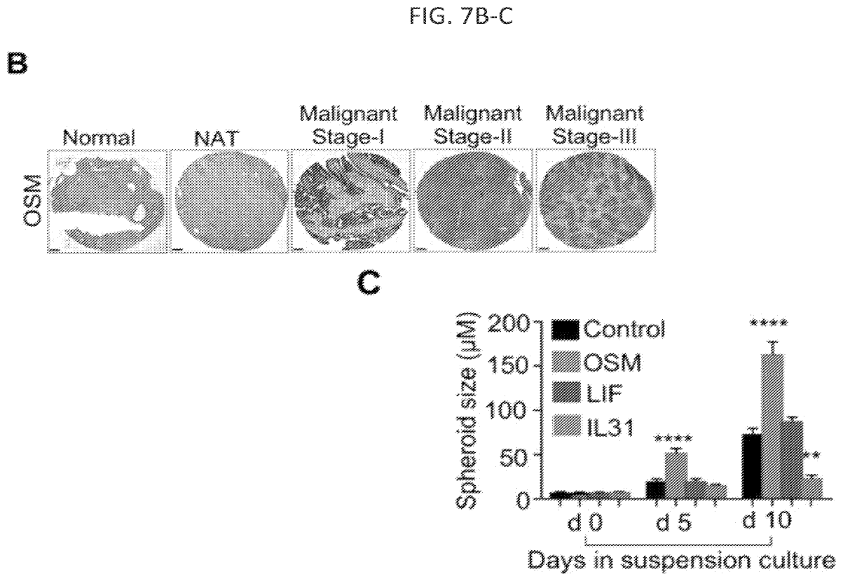

Gene Set Enrichment Analysis (GSEA) (Liberzon et al., 2015) further showed that high levels of OSM and OSMR are associated with functional annotation marks such as epithelial-to-mesenchymal transition and IL6/JAK/STAT3 signaling pathways in the TCGA ovarian cancer cohort (FIG. 7a). In conjunction, immunohistochemistry (IHC) using tissue microarrays (TMAs) consisting of 110 ovarian tumors (Table-1) exhibited high OSMR expression in the high-grade serous ovarian cancer patient samples compared to the low-grade serous ovarian cancer. The results also indicated that OSMR is highly expressed in the malignant stage I, II and III ovarian cancer tissues compared to normal and NAT tissues whereas no stagewise difference in OSM expression in ovarian cancer tissues (FIG. 7b) was observed. It was also found that OSM increased the spheroid forming ability and size of ovarian cancer spheroids compared to IL31 and LIF in 3-dimensional cultures (FIG. 7c). Importantly, OSM stimulation prolonged the phosphorylation at both Y705 and S727 moieties of STAT3 compared to LIF and IL31 in HEYA8 ovarian cancer cells. In conjunction, OSM promoted the growth of HEYA8 cancer cells rapidly compared to LIF and IL31 treatment in vivo.

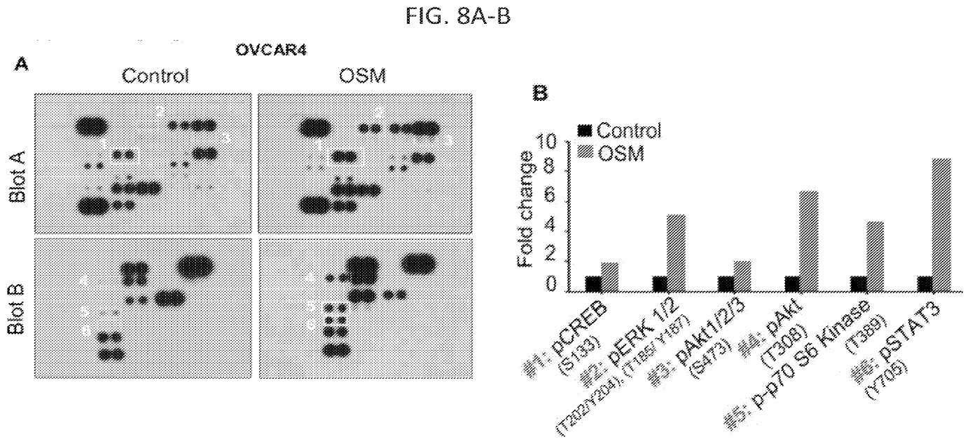

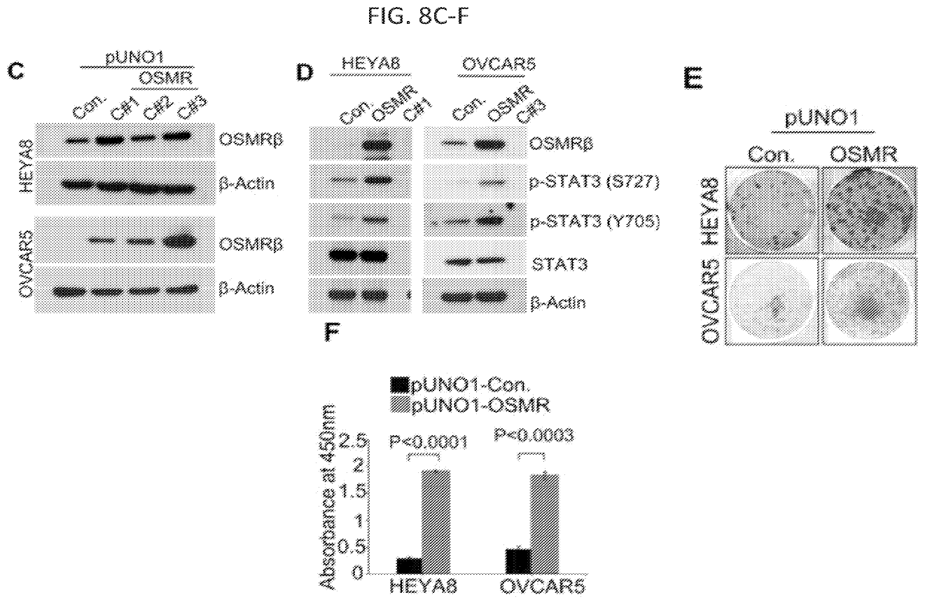

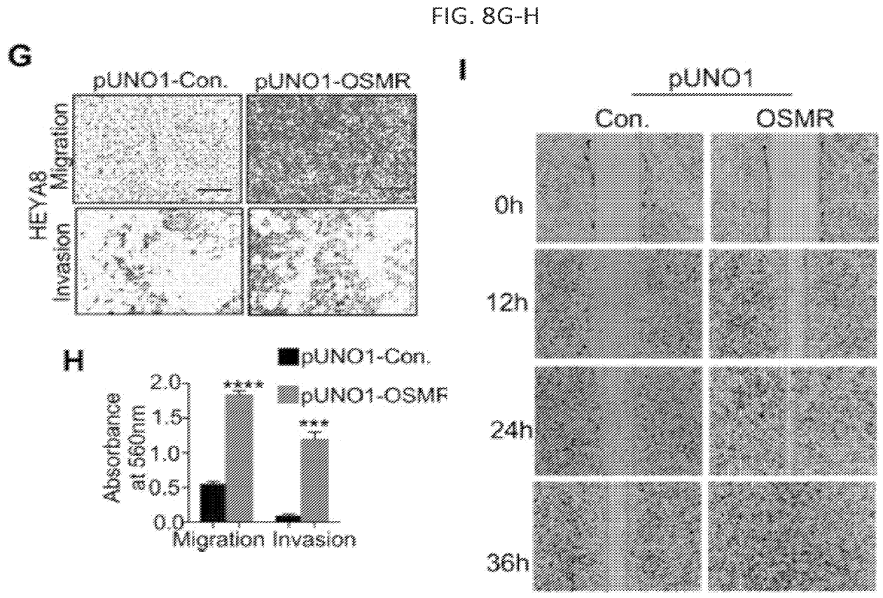

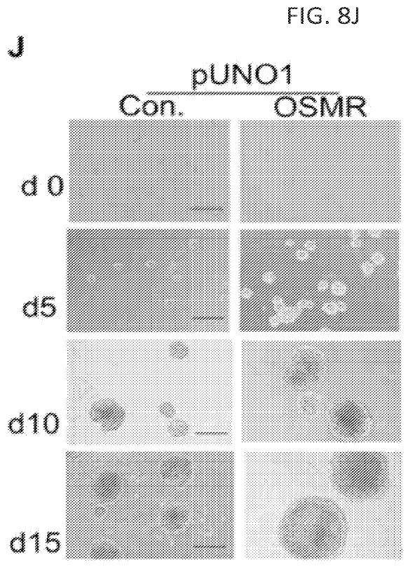

To investigate the downstream effects of OSMR activation upon OSM in ovarian cancer cells, the inventors stimulated OVCAR4 cells which express high levels of OSMR with recombinant human OSM and performed phospho-proteomic array and determined the phosphorylation of 45 proteins. In this assay, that the results indicated OSM stimulation increased the phosphorylation of several key proteins including CREB, ERK, STAT3, Akt, p70s6kinase, where pSTAT3-Y705 was the most upregulated phosphoprotein (FIGS. 8a and 8b). The oncogenic effects of OSMR were characterized by overexpressing OSMR in HEYA8 and OVCAR5 ovarian cancer cells using the pUNO1-OSMR plasmid. First, immunoblots were prepared using lysates of cells overexpressing OSMR and a substantial increase in the phosphorylation of Y705 and S727 moieties of STAT3 upon OSMR overexpression was observed (FIGS. 8c and 8d). OSMR also promoted colony formation, migration, invasion, wound healing ability and the spheroid forming capability in both cell lines (FIGS. 6e to 6j).

### Knock Down of OSMR Reduced Oncogenic Characteristics and Inhibited the Growth and Metastasis of Ovarian Cancer Cells

Next, it was determined if the knockdown of OSMR reduce the phosphorylation of STAT3 and the growth, migration and invasion of cancer cells. The results indicated that shRNA-mediated silencing of OSMR reduced phosphorylation of STAT3 at S727 and Y705 moieties. The inventors next determined if OSMR is required for the effects of OSM. In contrast to the effect of OSM in control cells, OSM stimulation could not activate the phosphorylation of STAT3 in the cells that were stably knocked down with shOSMR. Similarly, OSM stimulation did not induce any effect on spheroid formation, colony forming ability, cell migration or invasion in the cells that were knocked down with shOSMR, which again confirmed that OSMR is required for the effects of OSM.

HEYA8 tumors rarely develops ascites in mice; thus, a murine ovarian cancer cell line BR-Luc which develops ascites was used to study the effect of OSMR on OSM levels in ascitic fluid. To do this, BR-Luc murine cell lines that were stably depleted for OSMR using shOSMR or the control cells were injected intraperitoneally in syngeneic FVB mice (n=6/group). Ascites fluid was collected after 6-weeks of injection. Peritoneal wash was performed using 3 ml of PBS in the mice exhibit either low or no ascites, then OSM levels were determined by ELISA. Notably, the depletion of OSMR resulted into no ascitic fluid or very little ascites compared to the control group. To compare the levels of OSM in peritoneum, the inventors quantitated OSM either in the ascites fluid or in the peritoneal wash of those mice with no ascites and found that the mice bearing OSMR-depleted cells expressed poor amount of OSM in the peritoneal wash. In conjunction with the single cell analysis of clinical samples of ovarian cancer, the inventors found that the macrophage populations compared to epithelial cells and fibroblast expressed high levels of OSM in both peritoneal wash and ascites fluid of BR-Luc tumor bearing mice. Taken together, these results demonstrate that OSMR-depletion inhibited STAT3 phosphorylation, OSM levels and subsequent tumor growth.

To determine the effects of loss of expression of OSMR on ovarian cancer growth in vivo, luciferin labeled shControl-HEYA8 cells or shOSMR-HEYA8 cells were injected intraperitoneally in nude mice (n=7 mice/group) and the growth of cancer cells was monitored by bioluminescent imaging using in vivo imaging system (IVIS) up to 5 weeks. IVIS imaging in live animals showed that OSMR knockdown inhibited ovarian cancer cell growth by approximately 70% specifically in the last two time points. In conjunction, it was also found that silencing of OSMR markedly reduced the tumor weight and tumor burden as well as incidence of metastasis at various organ sites including omentum, peritoneum, perihepatic, perisplenic and pelvic sites. Consistent with the results that OSMR knockdown inhibited the ovarian cancer growth, IHC and immunoblot analysis showed that the stable knockdown of OSMR in these mice significantly decreased the cell proliferation marker Ki67 and increased the levels of apoptosis marker cleaved caspase-3. In addition, the inventors found that the loss of OSMR expression resulted a loss in the levels of phosphorylated STAT3 compared to sh-control. Notably, loss of OSMR also led to a decrease in the levels of proliferation marker PCNA and anti-apoptotic markers BCL-xl and BC12 compared to the respective controls. Next, the levels of secreted OSM in serum collected from the mice bearing HEYA8-control or HEYA8-shOSMR tumors were checked and a significant reduction in the OSM levels in the serum collected from the mice bearing HEYA8-shOSMR tumors was observed compared to the control. Taken together, the results demonstrate that silencing of OSMR inhibited the levels of proteins reduced the growth and metastasis of ovarian cancer cells both in vitro and in vivo.

Development and Screening of Anti-OSMR Antibodies that Inhibit the Growth of Ovarian Cancer Cells

A phage displayed single-chain variable fragment (scFv) antibody library was panned for binding activity to the extracellular domain of OSMR and all the positive scFv antibody clones which binds to recombinant OSMR upon screening in antigen-specific binding hits by ELISA were selected. The scFv clones that exhibited high binding affinity to OSMR were then converted to full-length IgG1 antibodies.

All the positive 26 full-length antibody clones, that have markedly high binding affinity towards extracellular domain of OSMR were identified and screened for their effect on ovarian cancer cell viability after treating the cancer cells with 10 μg/mL of antibody in the presence of OSM at multiple time points up to 48 h. WP1066, a potent inhibitor of STAT3 was used as a positive control and control IgG as an Isotype control. In the screening, it was observed that three antibodies named B14, B18, and B21 are the most potent antibodies that induce apoptosis in OVCAR4 cells even when grown in the presence of OSM. Next, the cell viability upon B14, B18, and B21 antibody treatment in OVCAR4 cells was determined using CCK8 cell viability assay and found that the IC50 of B14, B18, and B21 antibody clones are ˜10 μg/mL. Then, the binding affinity of these antibodies to cell surface human OSMR protein was analyzed by ELISA and found that antibodies B21 and B14 exhibited an effective concentration 50% (EC50) at 1.45 and 1.17 nM for B14 and B21 antibody, respectively. Unexpectedly, B18 antibody clones did not show any specific binding to OSMR. The B14 and B21 antibodies were selected to further characterize their effects on cellular signaling, tumor growth and metastasis. Importantly, both B14 and B21 treatment reduced the levels of phosphorylation of pSTAT3 (S727 and Y705), pAkt, p70s6Kinase, WNK1, PYK2, RSK1/2/3 and PLC-1 proteins in phospho-protein kinase array, where B21 was more effective on inhibiting those oncogenic kinases. It was also noticed that B21 antibody upregulated the levels proteins which cause cell cycle arrest and cell death such as TRAIL R1/DR4 and TRAIL R1/DR5, P21, BAX, p27/Kip1, and cleaved caspase 3; whereas inhibited the levels of pro-survival proteins such as BC12 and BClxL. In contrast, B14 antibody upregulated only p27 and cleaved caspase-3 in at a selected time point, whereas it reduced the levels of BCL2 and BCLxL more than B21 antibody. These findings were further corroborated by Annexin V FITC/PI assay using flow cytometry, which also showed a significant increase in early and late apoptotic cells after treatment with B14 and B21 anti-OSMR antibodies in the presence of OSM for 16 h.

Next, the inhibition of several key oncogenic kinases that was observed in the protein kinase array was validated by performing immunoblot using the lysates of HEYA8 and OVCAR4 cells that were treated with B14 and B21 mAbs. It was found that B14 and B21 treatments reduced phospho-STAT3 proteins and pro survival markers such as PCNA and BCL-xL; whereas improved the levels of apoptotic markers such as cytochrome c and p27.

Consistent with phospho-protein array data, immunoblots also showed that B14 and B21 antibodies reduced the level of phosphorylation of JAK1 (Y1034/1035) and JAK-2 (Y1007/1008), p85 subunit of PI3K (Y458), AKT (S473) and ERK (T202/Y204).

To further evaluate the anti-cancer effects of B14, and B21 antibodies in vitro, the effects of those antibodies on spheroid forming ability in HEYA8 cells and colony formation in both OVCAR4 and HEYA8 cells was examined and it was observed that B14 and B21 antibodies reduced the colony forming 3D morphogenesis abilities of OVCAR4 and HEYA8 cells. Notably, B14 and B21 mAbs inhibited the key markers of ovarian cancer stemness such as CD133 (Prominin), CD44, CD113 (c-KIT) and ALDH1 in HEYA8 cells. To confirm that the effects observed in ovarian cancer cells upon anti-OSMR antibody, are operated through OSMR and its downstream target STAT3, the inventors treated the most effective anti-OSMR antibody clone in HEYA8 cells that were knocked down for OSMR. As expected, B21 mAb was only effective in the control cells, whereas B21 mAb did not reduce the viability, spheroid formation ability, migration and colony formation of OSMR-depleted cells. In conjunction, levels of OSMR and phosphorylation of STAT3 were reduced upon B21 treatment in the control cells but was not altered in HEYA8-shOSMR cells.

### Anti-OSMR Antibody Abrogated the Dimerization of OSMR and Promoted the Internalization and Degradation of OSMR in Ovarian Cancer Cells

To investigate whether B14 and B21 monoclonal antibodies (mAbs) inhibit OSM-induced dimerization of OSMR with IL6ST, immunoprecipitated OSMR-IL6ST heterodimer complex was cross-linked after treating with anti-OSMR antibody or control IgG. Of note, it was found that B14 and B21 abrogated the dimerization between OSMR and IL6ST considerably in both HEYA8 and OVCAR4 cells. Of note, B14 and B21 mAb treatment did not affect the dimerization of OSMR with IL31RA induced by IL31 when OSMR was immunoprecipitated. Next, the inventors confirmed whether the treatment of B14 and B21 antibodies change the level of OSMR expression on cell membrane by on-cell Western assay. Here, the binding of B14 and B21 antibodies on the extracellular domain of OSMR was assessed in ovarian cancer cells by treating OVCAR4 and HEYA8 cells with control IgG, B14 or B21 in the presence of OSM for 24 h. Cells were then fixed and immunostained using a second and a commercially available anti-OSMR antibody labelled with IR Dye-680RD (red fluorescence) antibody and quantitated the level of OSMR on cell surface. In this assay, it was found that the treatment of B14 and B21 mAbs considerably reduced the presence of intact OSMR on the surface of both HEYA8 and OVCAR4 ovarian cancer cells, potentially due to the internalization and degradation of OSMR.

Thus, it was decided to further confirm if the binding of B14 and B21 mAbs could mediate the internalization and degradation of OSMR by immunoblotting OSMR and its dimerizing partner IL6ST using the cytoplasmic and membrane fractions isolated from HEYA8 ovarian cancer cells, which were treated with B14 and B21 antibodies in the presence of OSM. Surprisingly, the results showed that both B14 and B21 antibody promoted the internalization of OSMR to cytoplasm. It was then validated the antibody mediated OSMR internalization in a complimentary approach, where the Incucyte Live cell Analyzer was employed to monitor internalization of OSMR from cell surface to cytoplasm in real time. Here B14 and B21 mAbs that were pre-labeled with pH sensitive Incucyte FabFluor red reagent were used. In contrast to the extracellular pH at ˜7.4 (neutral pH), the FabFluor red labeled antibody produce red fluorescence when OSMR-labeled antibodies internalized into the cytoplasm or when localized to endosomes or lysosomes where the pH is acidic (˜6.3-4.7). B14 and B21 antibodies reduced the quantity of intact OSMR on cell surface and promoted its internalization. The FabFluor red labeled antibody-based assay demonstrated that both B14 and B21 antibodies induced internalization of OSMR from cell surface to cytoplasm ˜12 h after treatment in both HEYA8 and OVCAR4 cells.). Next, the inventors performed confocal microscopy on OVCAR4 cells that were treated with control IgG, B14 and B21 in the presence of OSM and immunostained with OSMR and LAMP1 (lysosomal marker). It was determined that B14 and B21 antibody treatments as compared to the control IgG promoted internalization of OSMR into cytoplasm and colocalization with LAMP1 as an indication of lysosomal degradation. Taken together, the results demonstrate that the treatment of both B14 and B21 anti-OSMR antibodies are strong antagonists to inhibit the oncogenic actions of OSMR mediated through its dimerization with IL6ST.

### In Vivo Delivery of Anti-OSMR Antibodies Reduced the Growth and Peritoneal Spread of Ovarian Cancer Cells

To evaluate the therapeutic effects of B14 and B21 anti-OSMR mAbs in vivo, the inventors injected in vitro validated anti-OSMR antibodies in athymic nude mice in which HEYA8 ovarian cancer cells were inoculated intraperitoneally with stably expressing luciferase reporter. Mice were treated with either control IgG, B14 or B21antibodies (10 mg/kg body weight) 7^{th} day after cancer cell inoculation intraperitoneally twice a week. Because the mouse derived OSM does not bind to the human OSMR (Adrian-Segarra et al., 2018), these mice were supplemented with recombinant human OSM (250 ng/kg body weight) twice/week intraperitoneally along with B14 and B21 antibody treatment. All the mice were monitored for the growth of cancer cells by bioluminescence imaging for five weeks. The treatment of exogenous OSM promoted the growth of ovarian cancer cells in vivo. In agreement with the in vitro finding, treatment of B14 and B21 antibodies reduced the overall burden of cancer cells, number of tumor nodules and incidence of metastasis compared to mice that were either treated with control IgG antibody alone or the mice received exogenous OSM along with control IgG. Notably, the survival analysis demonstrated that the mice bearing HeyA8 cells treated with B14 and B21 exhibited a better overall survival (log rank test p-value <0.0001) with a median survival of ˜60 days and more than 100 days respectively as compared to Isotype control IgG treated mice. In contrast, OSM stimulated mice along with control IgG antibody exhibited poor survival with median survival of 29 days as compared to Control IgG treated group alone. Immunohistochemistry analysis and/or Western blotting using the cancer tissues collected from, showed that B21 antibody treatment was more effective than B14 antibody in reducing OSMR, pSTAT3 expression, proliferation marker Ki67 and anti-apoptotic marker BCLxl as compared to control IgG with and without OSM treated mice. It was also found that B21 antibody treatment upregulated the levels of cell death marker cleaved caspase-3 in the cancer tissues as compared to OSM stimulated group, or when compared to B14 antibody group or control IgG group with and without OSM. Of note, the inventors did not find any unfavorable toxicity in the mice when treated with these antibody clones as exemplified by no significant change in body weight, alanine aminotransferase (ALT), aspartate aminotransferase (AST), bilirubin, albumin, creatine kinase, total protein levels and the histopathology of organs (kidney, liver, lung, heart, brain and spleen) in all the treatment groups.

Earlier studies of the OSMR family gene network were primarily focused on the signaling mechanisms dysregulated by OSM and/or OSMR for oncogenesis (Richards, 2013; Tanaka and Miyajima, 2003). However, the potential of developing OSMR as a therapeutic target was not sufficiently explored. According to the present disclosure, the inventors have determined the role of all IL6 family receptors in ovarian cancer and found that OSMR is an important therapeutic liability compared to other members of IL6 family receptors in ovarian cancer. The effects of all three ligands of OSMR family receptors such as OSM, IL31 and LIF on the kinetics of STAT3 phosphorylation were evaluated and it was found that OSM provides robust oncogenic signaling by prolonged activation of STAT3 compared to IL31 and LIF.

Using the power of single-cell sequencing according to the present disclosure, it is now known that OSMR is expressed predominantly in ovarian cancer cells and in cancer-associated fibroblasts compared to other cells in the tumor microenvironment. Without being bound by theory, this study suggests two paradigms, which is critical for the growth and progression of ovarian cancer cells: (i) a paradigm of oncogenic addiction operating in cancer cells which depends on elevated levels of OSMR and its interaction with its ligand OSM; and (ii) a paradigm of downstream oncogenic signaling operating through OSMR and is mediated by its dimerization with IL6ST upon OSM binding. Therefore, OSMR serves as an attractive target for abrogating oncogenic signaling, which could be executed by preventing the dimerization of OSMR with IL6ST and by promoting its internalization and degradation in cancer cells. One possible treatment strategy that may improve the outcome of ovarian cancer is the use of monoclonal antibodies (mAbs) that selectively target tumor cells with less affinity in non-tumor cells. In contrast to hematological cancers and solid malignancies such as breast and colorectal cancer, mAb-based therapy has not been proven to be effective for the treatment of ovarian cancer. Therefore, with the goal of treating ovarian cancer patients as a feasible therapeutic approach, the inventors developed a set of anti-OSMR antibodies and tested the efficacy of these antibodies in inhibiting the oncogenic signaling mediated through STAT3 and tumor cell growth and metastasis both in vitro and/or in vivo.

The present inventors found that the dimerization of OSMR with IL6ST is a critical step for OSM-induced signaling cues for tumor growth and progression. Thus, an agent that could abrogate the binding of OSM to OSMR and its dimerization with IL6ST could inhibit tumor progression. Notably, the antibodies of the present disclosure, such as B14 and B21 clones, for example, were capable of blocking OSM-induced dimerization of OSMR with IL6ST. Several mAbs have been developed and approved by the FDA for solid tumors. One of them being the widely used Trastuzumab (a.k.a. Herceptin).

Similar to the antibody of the present disclosure in terms of inhibiting heterodimerization of receptors, Trastuzumab is a humanized monoclonal antibody raised against the extracellular domain of HER2 (ERBB2) and is known for inhibiting the ligand-independent hetero-dimerization between ERBB2 and other EGFR family members by binding to the extracellular domain of HER2 (ERBB2) (Nahta et al., 2004). Trastuzumab was found to be effective in inhibiting the oncogenic signaling in HER2 expressing cancer cells and is widely using in the clinic to treat HER2-positive metastatic breast cancer patients when administered alone or in combination with chemotherapy (Burris et al., 2011; Nahta et al., 2004). Use of anti-OSMR monoclonal antibodies to treat those ovarian cancer patients with high OSMR levels should offer potential benefits such as avoiding the cytotoxic side effects in normal tissue caused by traditional chemotherapeutic agents or by nonselective targeting of cancer cells.

It is reported that antibodies bind to transmembrane receptors, block their binding to ligands, and inhibited tumor growth via receptor internalization and degradation (Ben-Kasus et al., 2009). Surprisingly, the inventors discovered that binding of B14 and B21 antibody to OMSR receptors blocked the OSMR dimerization but induced its internalization potentially by recognizing endocytic machinery, then sorting into lysosomes for degradation. These effects culminated into a reduction in phosphorylation and activation of downstream effectors such as STAT3, PI3K-Akt-mTOR and MEK-ERK proteins, which are important for tumor growth and progression. Several therapeutic antibodies have been implicated in the induction of apoptosis via intrinsic (or mitochondrial) pathway, leading to cytochrome release from mitochondria, downregulation of anti-apoptotic Bcl-2 family proteins, and upregulation of cyclin-dependent kinases (CDK) inhibitors (Ben-Kasus et al., 2007). The pro-apoptotic activity of Trastuzumab has been attributed to inhibition of constitutively activated MAP-kinase and Akt pathways downstream to ERBB2, and to TRAIL-induced apoptosis (Cuello et al., 2001). Similar to these findings, it was observed that B14 and/or B21 antibody clones of the present disclosure inhibited the levels of BC12, and upregulated the levels of p27, cleaved caspase-3, TRAIL receptors and cytochrome c. Likewise and as expected, the treatment of B14 and/or B21 reduced the growth and peritoneal spread of ovarian cancer cells in vivo, where the treatment inhibited the levels of OSMR, phosphorylated STAT3, BCL-xL and induced the levels of cleaved caspase-3.

OSMR is highly expressed in ovarian cancer cells (Geethadevi et al., 2021).

In addition, OSM is involved in oncogenic signaling activation for prolonged period and ovarian cancer growth. OSMR knockdown reduces the growth and seeding of ovarian cancer cells in vivo (Geethadevi et al., 2021). Moreover, monoclonal antibody (mAb) of OSMR abrogates OSM-mediated oncogenic characteristics by blocking dimerization of OSMR and anti-OSMR antibodies abrogate heterodimerization of OSMR with IL6ST and induce internalization and degradation of OSMR (Geethadevi et al., 2021). In addition, anti-OSMR antibodies reduced OSM mediated tumor growth and metastasis and improved overall survival rate of mice bearing ovarian cancer.

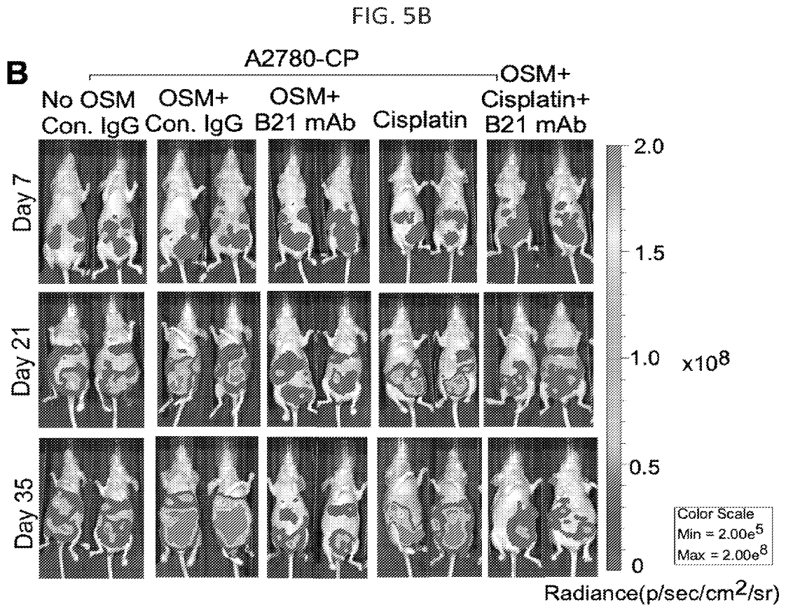

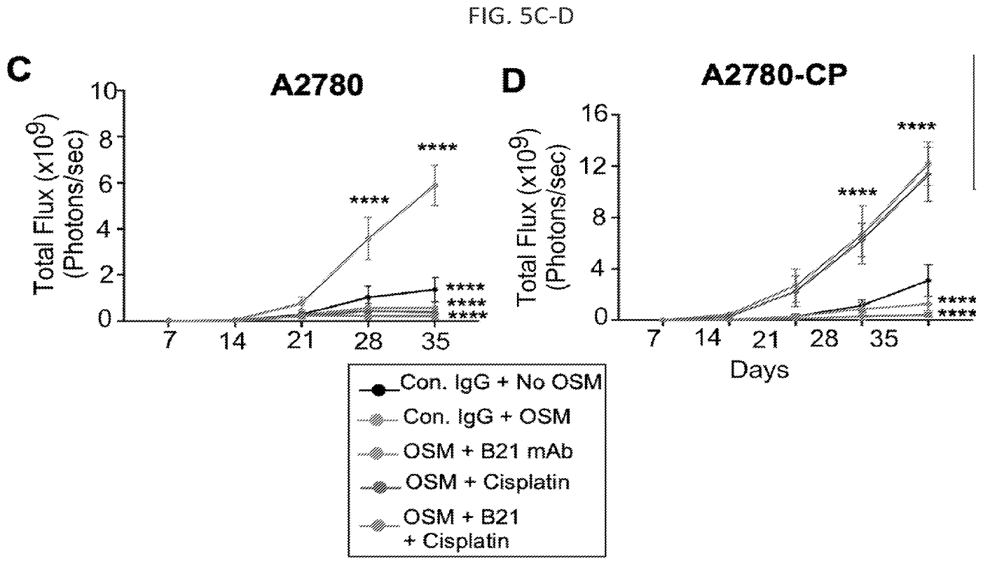

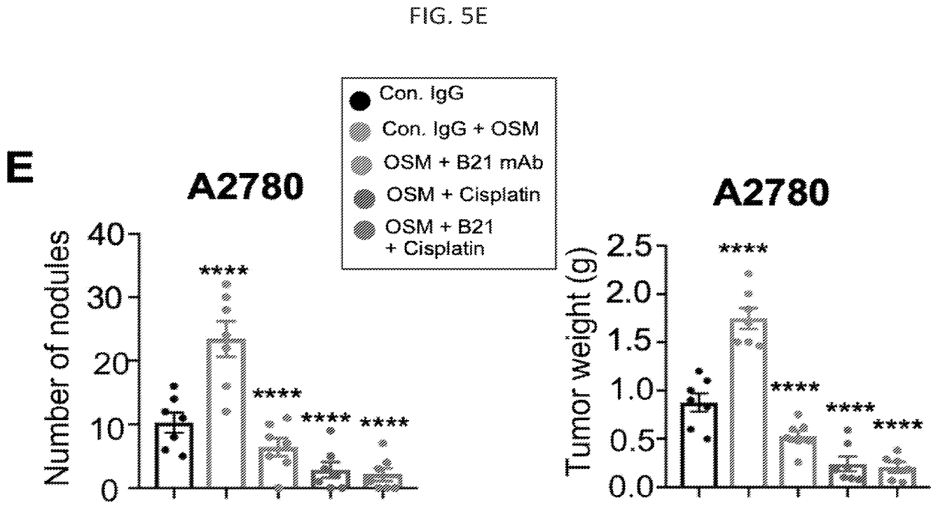

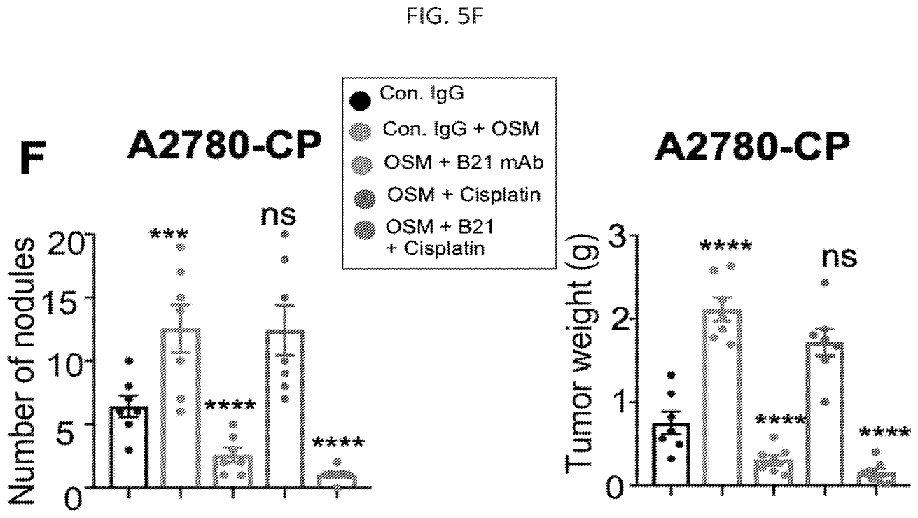

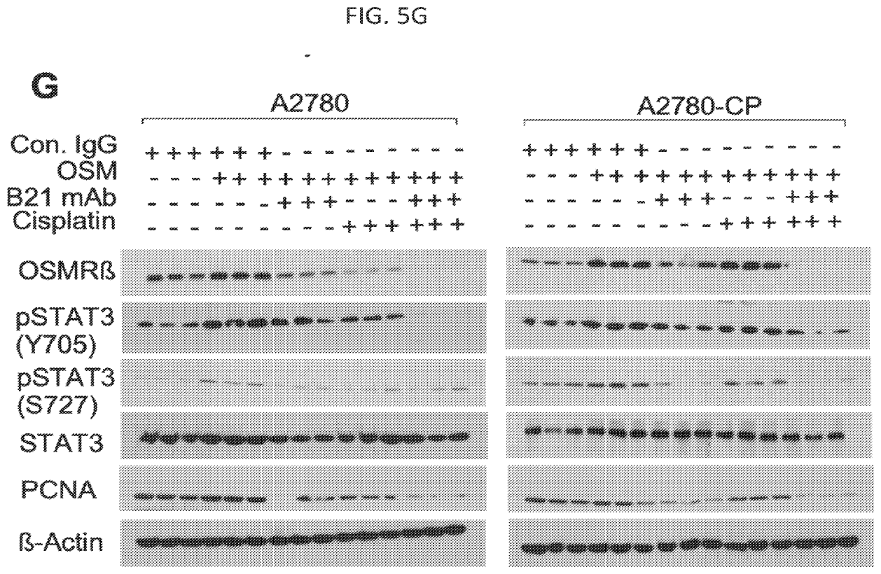

FIG. 5.

It is now known that target-specific inhibition of OSMR using anti-OSMR antibodies abrogates cisplatin resistance as demonstrated, for example, by the following observations.

### Oncostatin M and its Receptor OSMR are Upregulated in Cisplatin-Resistant Ovarian Cancer Cells.

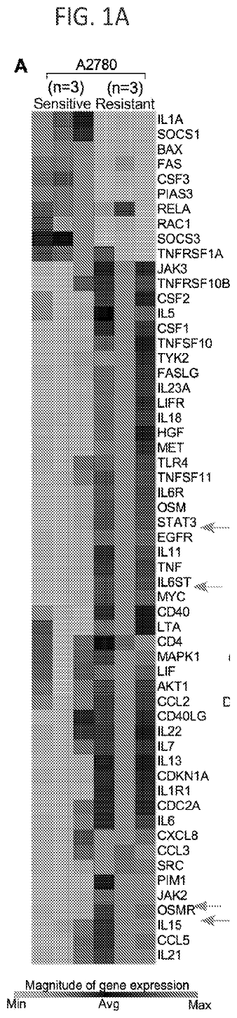

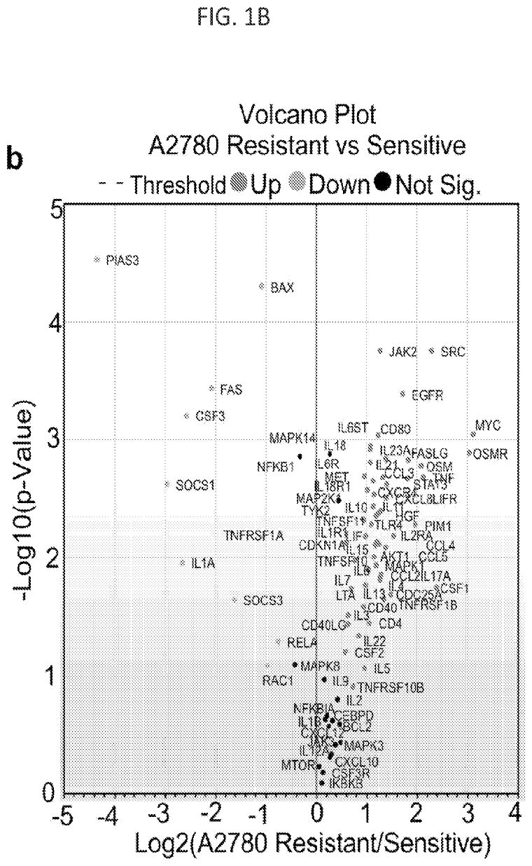

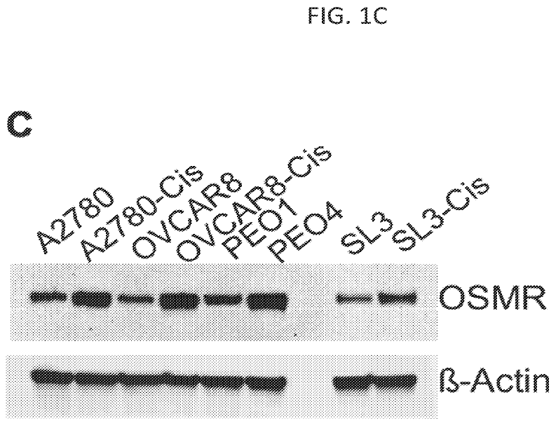

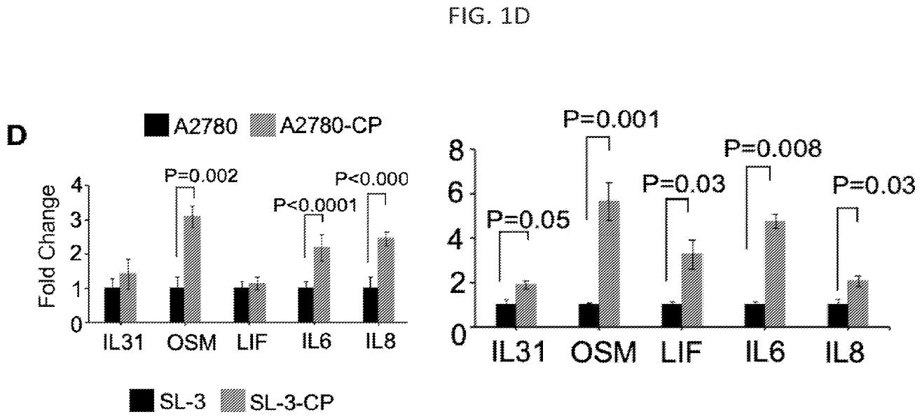

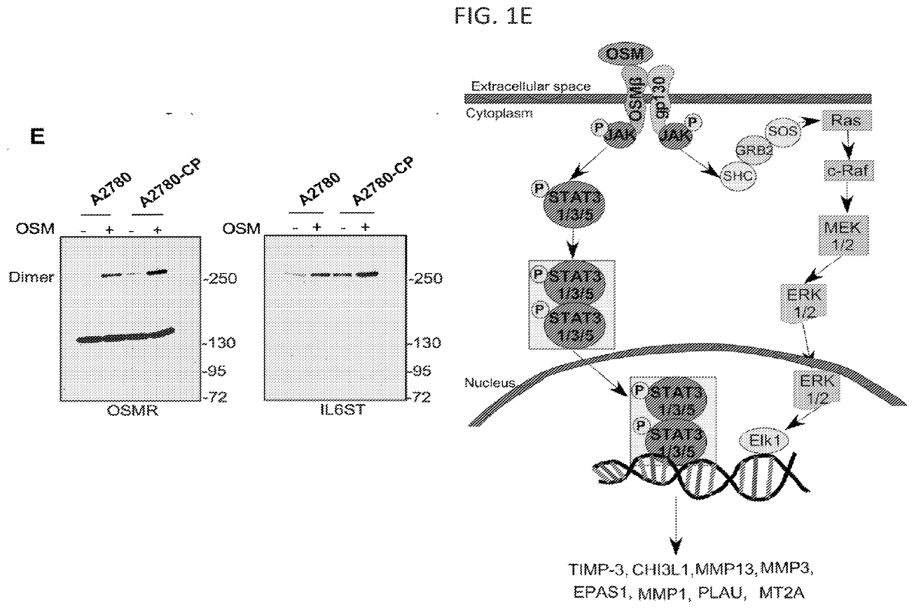

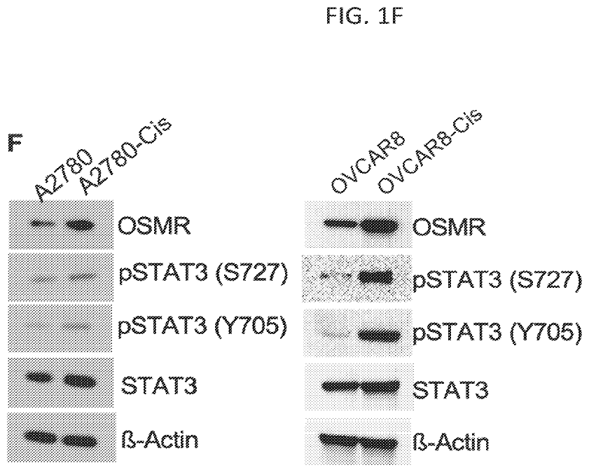

To identify the inflammatory cytokines upregulated in the aggressive and chemo-resistant versions of ovarian cancer cells, qPCR array was done for all cytokines, interleukins, chemokines, their receptors and downstream effector genes in both the parent and cisplatin-resistant A2780 ovarian cancer cells (FIG. 1.A). In this array, the inventors identified that out of 84 genes, 66 genes were differentially expressed in both groups analyzed after a cut off value of ≥1.5-fold change difference in both directions (FIG. 1A).

Among the 66 differentially expressed genes, it was determined that MYC, OSMR, CSF1, SRC, TNF, OSM, EGFR, FASLG, PIM1 and STAT3 were the top-10 genes which are upregulated in the cisplatin resistant A2780 cells. In contrast, it was determined that only a few genes were downregulated in the cisplatin resistant A2780 cells and it was found that BAX, SOCS3, FAS, CSF3, ILIA, SOCS1, and PIAS3 are the top-down regulated genes (FIG. 1A). Strikingly, cisplatin-resistant version of A2780 cells express high levels of Oncostatin M (OSM), its receptor Oncostatin M receptor (OSMR) and its downstream effectors JAK2 and STAT3. In contrast, cisplatin-resistant version of A2780 cells expressed low levels of PIAS3 which is the protein inhibitor of activated STAT 3 (FIG. 1A and FIG. 1B). These data indicate that OSM-signaling is an important mechanism for the progression and metastasis of ovarian cancer. To further validate the expression of OSM or OSMR, protein levels in the platinum-resistant versions of ovarian cancer cells, PEO1 which are sensitive to cisplatin and PEO4, which are resistant to cisplatin and in a patient-derived endometrioid ovarian cancer cell line SL3 and A2780 (FIG. 1C) and identified an increase in OSMR and OSM protein levels in the cisplatin resistant ovarian cancer cell lines.

To further characterize the role of OSM, OSMR and its downstream effectors in the ovarian cancer, which are resistant to cisplatin. OSM belongs to IL-6 family ligands, which includes Interleukin-6 (IL-6), Interleukin-11 (IL-11), Interleukin-31 (IL-31), leukemia inhibitory factor (LIF), Oncostatin M (OSM), ciliary neurotrophic factor (CNTF), leptin (OB), cardiotrophin-1 (CT-1), novel neurotrophin-1/B cell stimulating factor-3 or cardiotrophin like cytokine (CLC), and neuropoietin (NP). It was confirmed the secreted levels of IL-31, OSM, IL-6 and LIF in the wild type and cisplatin-resistant version of A2780 and SL-3 by Multiplex cytokine ELISA and found that OSM was the most upregulated cytokine among IL-31, OSM, and LIF and even higher compared to IL6 and IL8 (FIG. 1D). OSM induced heterodimerization of its receptor OSMR with IL6ST (also known as gp 130) is an early event in the initiation and activation of downstream signaling pathway such as JAK-STAT pathway. To examine the role of OSM in initiating OSMR-IL6ST heterodimerization, a cross-linking dimerization assay was done in A2780 sensitive and A2780-CP in response to OSM stimulation then treated with a membrane-impermeable chemical crosslinker, BS3 (FIG. 1E). Further immunoprecipitation using specific anti-OSMR antibody which capture OSMR-IL6ST dimerized receptor complex yielded dimer and monomer of OSMR and dimer of IL6ST in both cells. It was noted that OSM-induced heterodimerization of OSMR was comparatively elevated in A2780-CP than A2780 sensitive, which could be the result of high expression of OSMR in A2780-CP cisplatin resistant cells (FIG. 1E). Evaluation of the phosphorylation levels of STAT3 in A2780 sensitive and A2780-CP and OVCAR8 sensitive and OVAR8-Cis and found that phosphorylation of STAT3 (Y705, S737 sites) in A2780-Cis and OVCAR8-Cis cell lines (FIG. 1F).

Treatment with Anti-OSMR Antibody Enhances Cisplatin Treatment in Ovarian Cancer Cells Both In Vitro and In Vivo

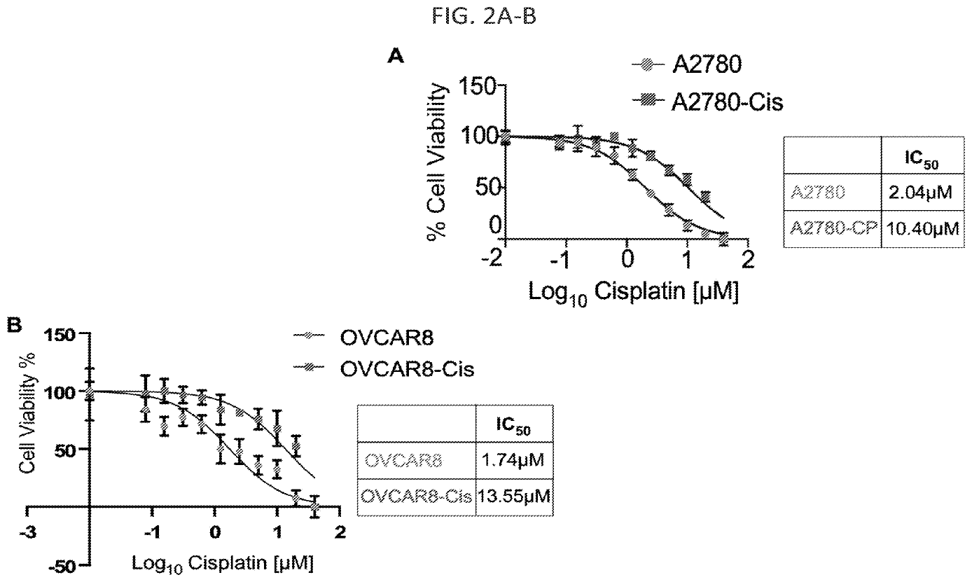

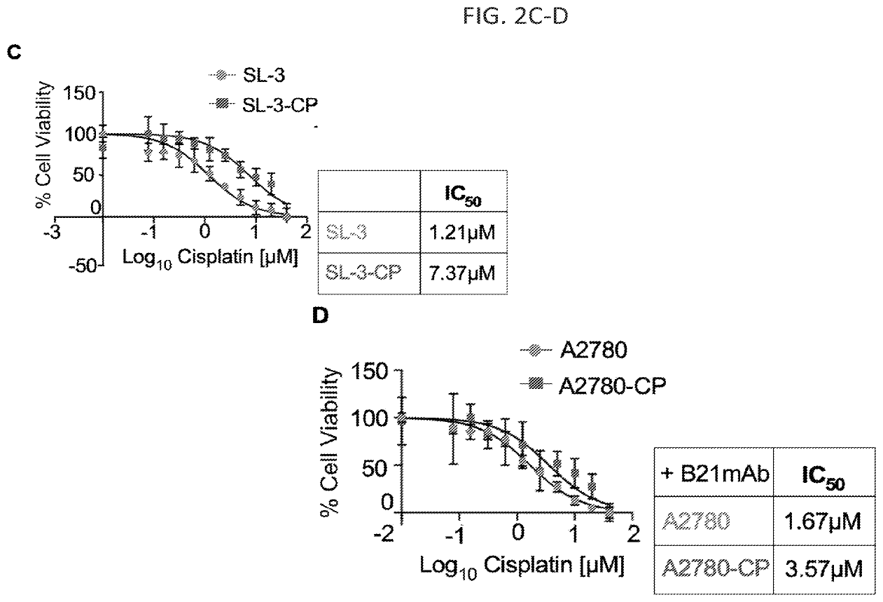

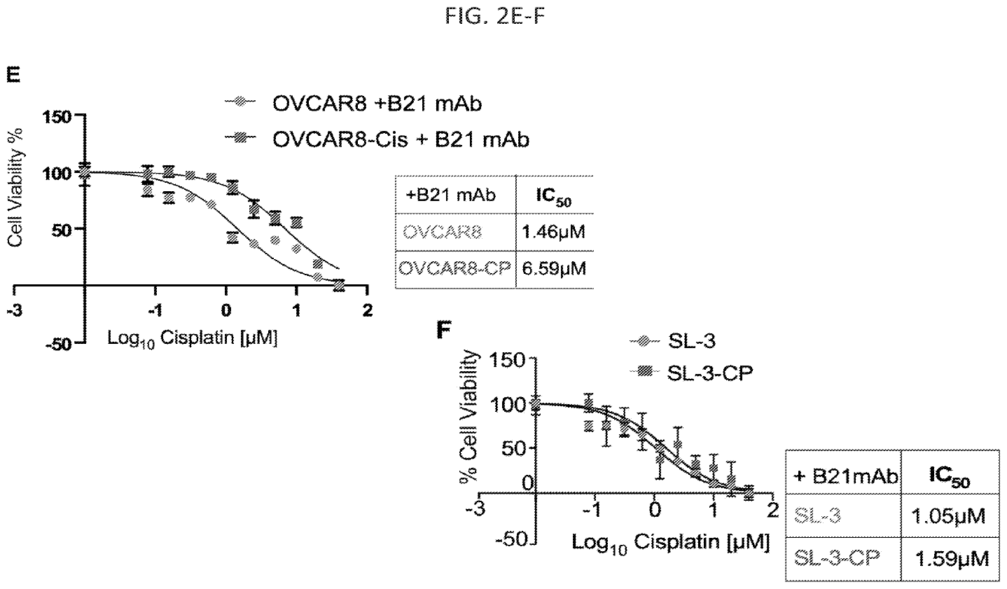

The data showed that OSMR and OSM are highly expressed in cisplatin-resistant ovarian cancer cells A2780-Cis, OVACR8-Cis and SL-3-Cis as compared to the sensitive cell lines (FIG. 1C). To evaluate whether B21 anti-OSMR antibody sensitizes cisplatin treatment in ovarian cancer cells, the inventors tested the effect of B21 alone or in combination with cisplatin. First, the effect of cell viability of B21 was determined in both parent and cisplatin-resistant versions of A2780, OVCAR8 and SL3 ovarian cancer cell lines. The IC50 of cisplatin in A2780-Cis, OVCAR8-Cis and SL-3-Cis was found to be 10.40 μM, 13.55 μM and 7.37 μM respectively, whereas the IC50 of cisplatin in the parental A2780, OVCAR8 and SL3 cell lines were 2.04 μM, 1.74 μM and 1.21 μM respectively (FIG. 2A-C). Notably, treatment of B21 antibody reduced the IC50 in cisplatin-resistant cells to 3.57 μM, 6.59 μM and 1.59 μM in A2780-Cis, OVCAR8-Cis and SL-3-Cis respectively, whereas B21 did not make any significant change in the IC50 of cisplatin in the cisplatin-sensitive parental version of cells (FIG. 2D-F).

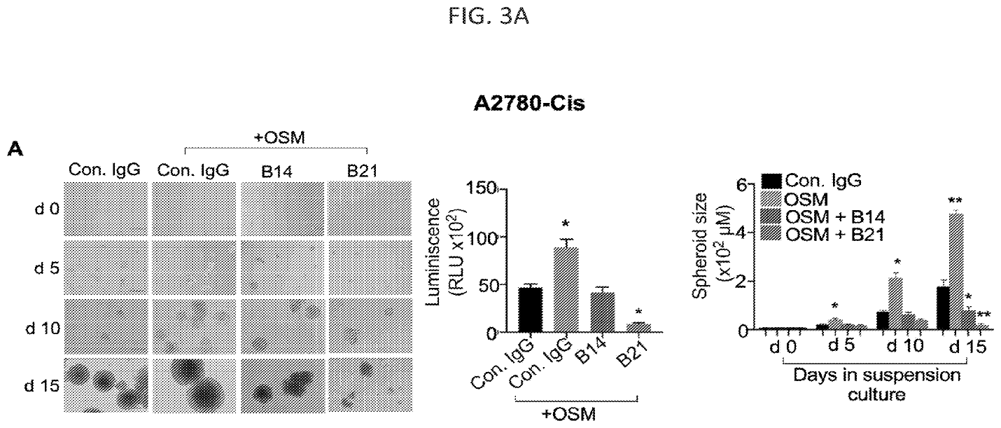

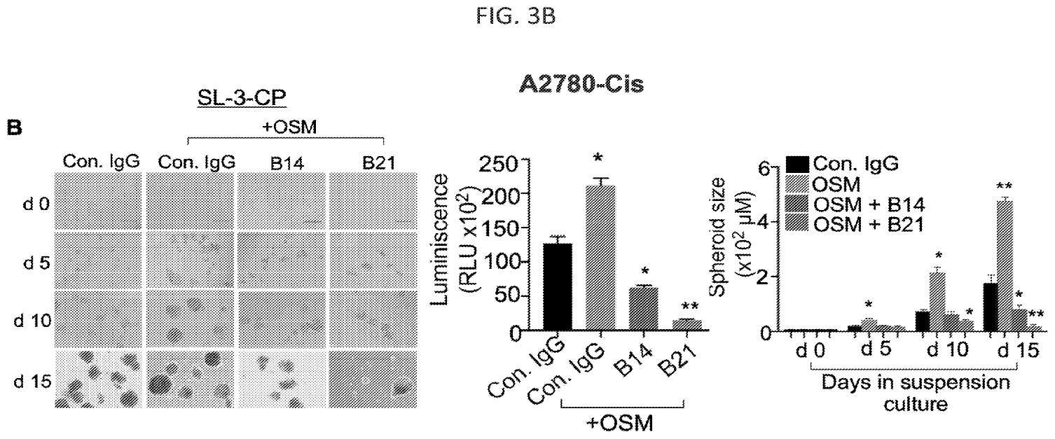

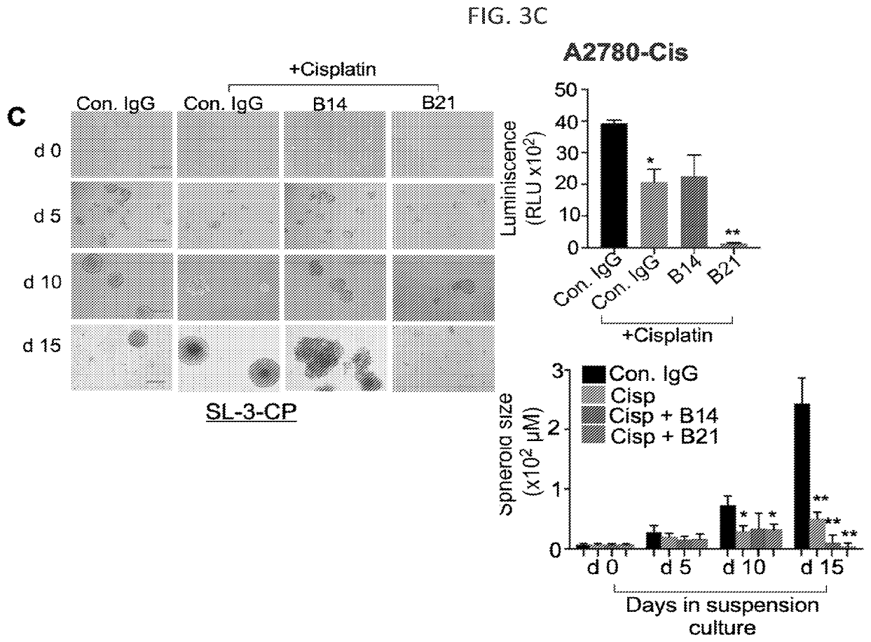

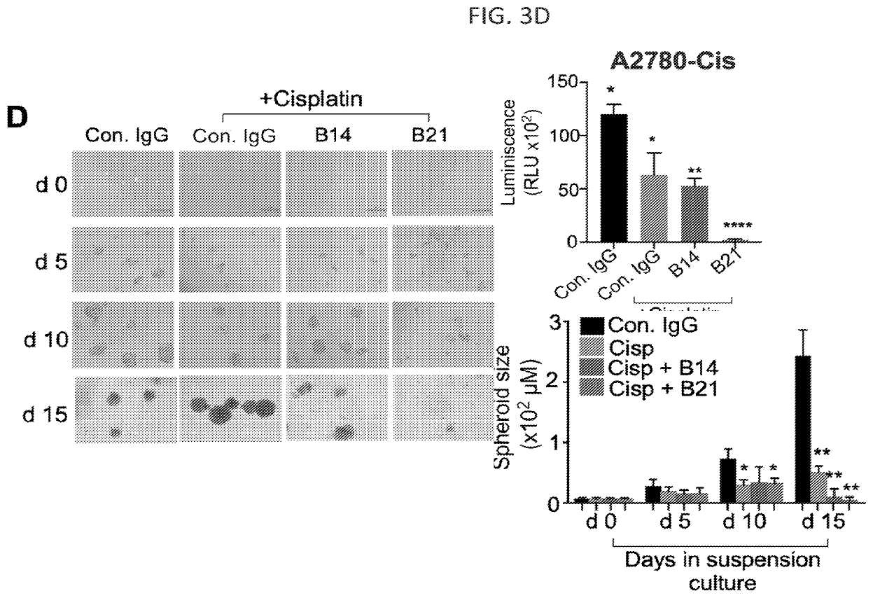

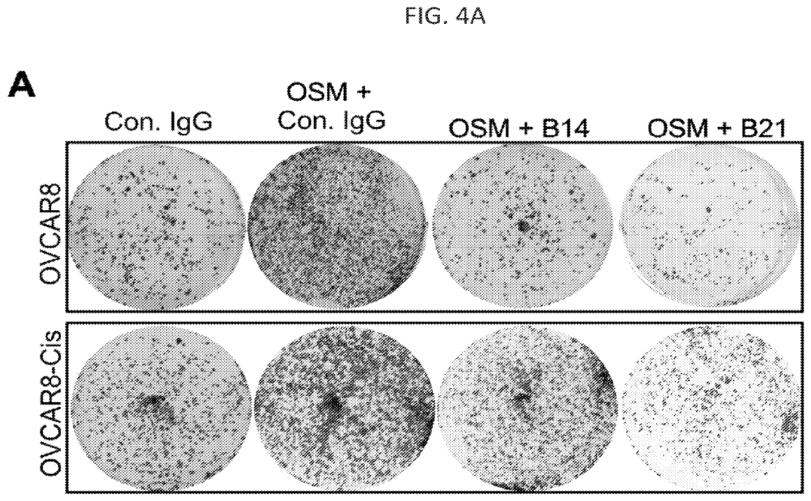

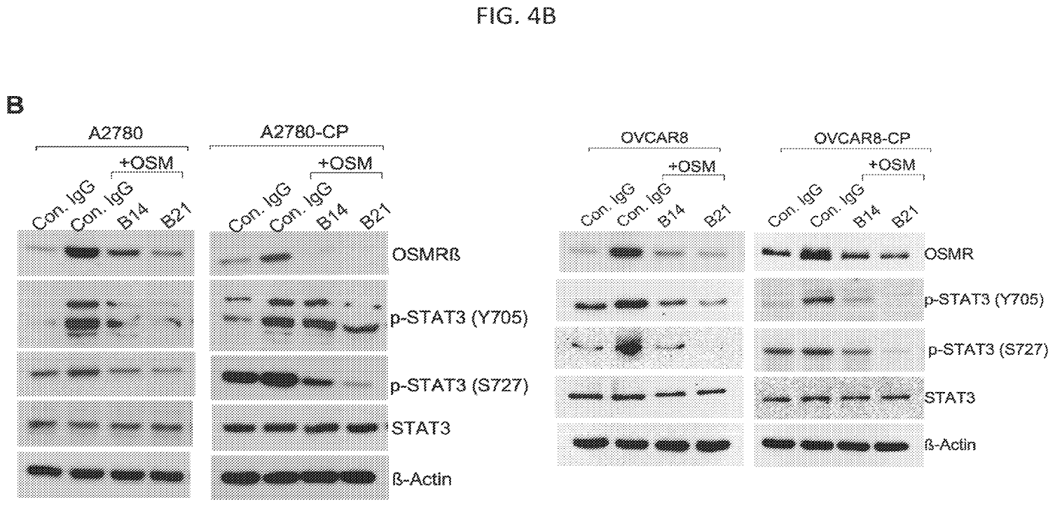

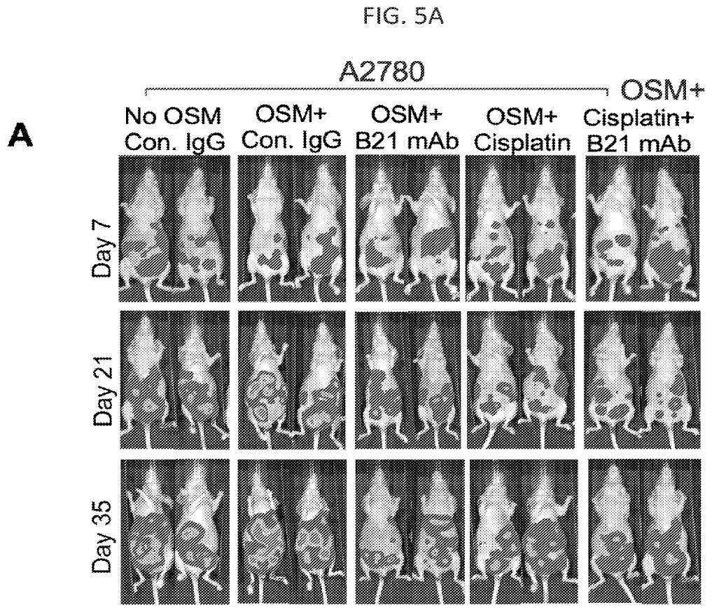

To characterize the effects of B21 antibody on the spheroid forming ability of A2780-Cis and SL-3-Cis in low-attachment plate for 15 days. Here, treated A2780-CP and SL3-CP cell lines were treated with B14 and B21 mAbs in the presence of OSM and found that B21 treated cells significantly reduced the tumor forming ability when monitored for 15 days as compared to B14 mAb and OSM treated alone or Control IgG (FIG. 3A-B). Next, B21 antibody was combined with cisplatin and it was observed that the combination significantly reduced the spheroid forming capacity of tumor cells and caused the disintegration of spheroids compared to spheroids treated with cisplatin alone (FIG. 3C-D). It was also determined the effect of B21 mAb on the cancer stemness or tumor initiating capacity (TIC) effect by performing the limiting dilution assay in both A2780-CP and SL-3-CP ovarian cancer cells in the presence of control IgG, cisplatin and con. IgG, and cisplatin along with B21 antibody at increasing cell number in low-attachment plate using ClonaCell suspension culture media. In this assay, it was determined that the B21 mAbs treatment significantly reduced sphere forming capacity ˜90% consistently in all the serial dilutions (FIG. 3E-F), suggesting that blocking of OSMR-signaling would inhibit TIC. Additionally, B14 and B21 mAbs, even in the presence of OSM, decreased the colony forming ability of OVCAR8 and OVCAR8-Cis (FIG. 4A). Interestingly, we found that B14 and B21 antibodies significantly decreased the OSMR expression and phosphorylation of pSTAT3 at Y705 and S727 in A2780 and SL3 resistant cell lines as compared to OSM treatment alone (FIG. 4B).

Given the effects of B21 mAbs on inhibiting the growth of ovarian cancer cells and sensitizing efficacy of cisplatin in vitro, to demonstrate the effects of B21 mAbs in vivo. A2780-sensitive or A2780-cisplatin resistant (A2780-Cis) cell lines that stably expressing luciferase reporter were injected intraperitoneally in athymic nude mice and tumor burden was monitored using bioluminescence once weekly (FIG. 5A). Mice were then randomly separated into five groups and treated with B21 mAb or with cisplatin (5 mg/kg body weight biweekly) alone or in their combination and the mice were stimulated with recombinant OSM. B21 mAb or cisplatin reduced the growth of A2780 ovarian cancer compared to the mice bearing A2780 ovarian cancer stimulated with OSM alone, whereas there is no significant decrease when B21 with cisplatin were combined (FIG. 5A and FIG. 5C). The A2780-CP cells exhibited an aggressive growth in nude mice as compared to the A2780-sensitive cells, where cisplatin alone did not inhibit the growth and metastasis of A2780-CP tumors (FIG. 5B and FIG. 5D). Importantly, B21 mAb treatment along with cisplatin further decreased the tumor weight than single treatment (FIG. 5E-F). immunoblots of tumor tissue extracted from the above groups were analyzed for the levels of OSMR and its downstream targets. There was a significant reduction of OSMR and phosphorylated STAT3 in the combination treatment of B21 with cisplatin in the A2780-CP tumors (FIG. 5G). The levels of PCNA expression decreased in A2780-CP treated with B21 alone and in combination with Cisplatin indicated an increased cell apoptosis as well as reduced cell proliferation in tumors treated with the combination of anti-OSMR antibody and cisplatin. These results demonstrate a potent synergistic effect when B21 anti-OSMR antibody combined with cisplatin to treat ovarian cancers particularly the aggressive cisplatin-resistant version.

### REFERENCES

Adrian-Segarra, J. M., Sreenivasan, K., Gajawada, P., Lorchner, H., Braun, T., and Poling, J. (2018). The AB loop of oncostatin M (OSM) determines species-specific signaling in humans and mice. J Biol Chem 293, 20181-20199.
Barger, C. J., Zhang, W., Hillman, J., Stablewski, A. B., Higgins, M. J., Vanderhyden, B. C., Odunsi, K., and Karpf, A. R. (2015). Genetic determinants of FOXMI overexpression in epithelial ovarian cancer and functional contribution to cell cycle progression. Oncotarget 6, 27613-27627.
Ben-Kasus, T., Schechter, B., Lavi, S., Yarden, Y., and Sela, M. (2009). Persistent elimination of ErbB-2/HER2-overexpressing tumors using combinations of monoclonal antibodies: relevance of receptor endocytosis. Proc Natl Acad Sci USA 106, 3294-3299.
Ben-Kasus, T., Schechter, B., Sela, M., and Yarden, Y. (2007). Cancer therapeutic antibodies come of age: targeting minimal residual disease. Mol Oncol 1, 42-54.
Bublil, E. M., Pines, G., Patel, G., Fruhwirth, G., Ng, T., and Yarden, Y. (2010). Kinase-mediated quasi-dimers of EGFR. FASEB J 24, 4744-4755.
Burris, H. A., 3rd, Tibbitts, J., Holden, S. N., Sliwkowski, M. X., and Lewis Phillips, G. D. (2011). Trastuzumab emtansine (T-DM1): a novel agent for targeting HER2+ breast cancer. Clin Breast Cancer 11, 275-282.
Butler, A., Hoffman, P., Smibert, P., Papalexi, E., and Satija, R. (2018). Integrating single-cell transcriptomic data across different conditions, technologies, and species. Nat Biotechnol 36, 411-420.
Chaluvally-Raghavan, P., Jeong, K. J., Pradeep, S., Silva, A. M., Yu, S., Liu, W., Moss, T., Rodriguez-Aguayo, C., Zhang, D., Ram, P., et al. (2016). Direct Upregulation of STAT3 by MicroRNA-551b-3p Deregulates Growth and Metastasis of Ovarian Cancer. Cell Rep 15, 1493-1504.
Chen, C., Gupta, P., Parashar, D., Nair, G. G., George, J., Geethadevi, A., Wang, W., Tsaih, S. W., Bradley, W., Ramchandran, R., et al. (2020). ERBB3-induced furin promotes the progression and metastasis of ovarian cancer via the IGFIR/STAT3 signaling axis. Oncogene 39, 2921-2933.
Coward, J. I., Middleton, K., and Murphy, F. (2015). New perspectives on targeted therapy in ovarian cancer. Int J Womens Health 7, 189-203.
Cuello, M., Ettenberg, S. A., Clark, A. S., Keane, M. M., Posner, R. H., Nau, M. M., Dennis, P. A., and Lipkowitz, S. (2001). Down-regulation of the erbB-2 receptor by trastuzumab (herceptin) enhances tumor necrosis factor-related apoptosis-inducing ligand-mediated apoptosis in breast and ovarian cancer cell lines that overexpress erbB-2. Cancer Res 61, 4892-4900.
Danneman P, Suckow M A, Brayton C, Suckow M A. The laboratory mouse. Boca Raton: Taylor & Francis; 2013. xvi, 234 p. p.
Demyanets, S., Kaun, C., Rychli, K., Pfaffenberger, S., Kastl, S. P., Hohensinner, P. J., Rega, G., Katsaros, K. M., Afonyushkin, T., Bochkov, V. N., et al. (2011). Oncostatin M-enhanced vascular endothelial growth factor expression in human vascular smooth muscle cells involves PI3K-, p38 MAPK-, Erk1/2- and STAT1/STAT3-dependent pathways and is attenuated by interferon-gamma. Basic Res Cardiol 106, 217-231.
Efremova, M., Vento-Tormo, M., Teichmann, S. A., and Vento-Tormo, R. (2020). CellPhoneDB: inferring cell-cell communication from combined expression of multi-subunit ligand-receptor complexes. Nat Protoc 15, 1484-1506.
Goldberg, R. M. (2005). Cetuximab. Nat Rev Drug Discov Suppl, S10-11.
Izar, B., Tirosh, I., Stover, E. H., Wakiro, I., Cuoco, M. S., Alter, I., Rodman, C., Leeson, R., Su, M. J., Shah, P., et al. (2020). A single-cell landscape of high-grade serous ovarian cancer. Nat Med 26, 1271-1279.
Jagadish, N., Parashar, D., Gupta, N., Agarwal, S., Purohit, S., Kumar, V., Sharma, A., Fatima, R., Topno, A. P., Shaha, C., and Suri, A. (2015). A-kinase anchor protein 4 (AKAP4) a promising therapeutic target of colorectal cancer. J Exp Clin Cancer Res 34, 142.
Jagadish, N., Parashar, D., Gupta, N., Agarwal, S., Suri, V., Kumar, R., Suri, V., Sadasukhi, T. C., Gupta, A., Ansari, A. S., et al. (2016). Heat shock protein 70-2 (HSP70-2) is a novel therapeutic target for colorectal cancer and is associated with tumor growth. BMC Cancer 16, 561.
Kanojia, D., Garg, M., Saini, S., Agarwal, S., Parashar, D., Jagadish, N., Seth, A., Bhatnagar, A., Gupta, A., Kumar, R., et al. (2013). Sperm associated antigen 9 plays an important role in bladder transitional cell carcinoma. PLOS One 8, e81348.
Kurosawa, T., Yamada, A., Takami, M., Suzuki, D., Saito, Y., Hiranuma, K., Enomoto, T., Morimura, N., Yamamoto, M., Iijima, T., et al. (2015). Expression of nephronectin is inhibited by oncostatin M via both JAK/STAT and MAPK pathways. FEBS Open Bio 5, 303-307.
Liberzon, A., Birger, C., Thorvaldsdottir, H., Ghandi, M., Mesirov, J. P., and Tamayo, P. (2015). The Molecular Signatures Database (MSigDB) hallmark gene set collection. Cell Syst 1, 417-425.
Maranto C, Udhane V, Jia J, Verma R, Muller-Newen G, LaViolette P S, et al. Prospects for Clinical Development of Stat5 Inhibitor IST5-002: High Transcriptomic Specificity in Prostate Cancer and Low Toxicity In Vivo. Cancers (Basel) 2020; 12
Murakami, M., Kamimura, D., and Hirano, T. (2019). Pleiotropy and Specificity: Insights from the Interleukin 6 Family of Cytokines. Immunity 50, 812-831.
Nahta, R., Hung, M. C., and Esteva, F. J. (2004). The HER-2-targeting antibodies trastuzumab and pertuzumab synergistically inhibit the survival of breast cancer cells. Cancer Res 64, 2343-2346.
Parashar, D., Geethadevi, A., Aure, M. R., Mishra, J., George, J., Chen, C., Mishra, M. K., Tahiri, A., Zhao, W., Nair, B., et al. (2019). miRNA551b-3p Activates an Oncostatin Signaling Module for the Progression of Triple-Negative Breast Cancer. Cell Rep 29, 4389-4406 e4310.
Phuchareon, J., McCormick, F., Eisele, D. W., and Tetsu, O. (2015). EGFR inhibition evokes innate drug resistance in lung cancer cells by preventing Akt activity and thus inactivating Ets-1 function. Proc Natl Acad Sci USA 112, E3855-3863.
Richards, C. D. (2013). The enigmatic cytokine oncostatin m and roles in disease. ISRN Inflamm 2013, 512103.
Romano, G., Chen, P. L., Song, P., McQuade, J. L., Liang, R. J., Liu, M., Roh, W., Duose, D. Y., Carapeto, F. C. L., Li, J., et al. (2018). A Preexisting Rare PIK3CA (E545K) Subpopulation Confers Clinical Resistance to MEK plus CDK4/6 Inhibition in NRAS Melanoma and Is Dependent on S6K1 Signaling. Cancer Discov 8, 556-567.
Rose-John, S. (2018). Interleukin-6 Family Cytokines. Cold Spring Harb Perspect Biol 10. Siegel, R. L., Miller, K. D., and Jemal, A. (2018). Cancer statistics, 2018. CA Cancer J Clin 68, 7-30.
Slyper, M., Porter, C. B. M., Ashenberg, O., Waldman, J., Drokhlyansky, E., Wakiro, I., Smillie, C., Smith-Rosario, G., Wu, J., Dionne, D., et al. (2020). A single-cell and single-nucleus RNA-Seq toolbox for fresh and frozen human tumors. Nat Med 26, 792-802.
Smyth, E. C., Tarazona, N., and Chau, I. (2014). Ramucirumab: targeting angiogenesis in the treatment of gastric cancer. Immunotherapy 6, 1177-1186.
Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550.
Taga, T., and Kishimoto, T. (1997). Gp130 and the interleukin-6 family of cytokines. Annu Rev Immunol 15, 797-819.
Tanaka, M., and Miyajima, A. (2003). Oncostatin M, a multifunctional cytokine. Rev Physiol Biochem Pharmacol 149, 39-52.
Torre, L. A., Trabert, B., DeSantis, C. E., Miller, K. D., Samimi, G., Runowicz, C. D., Gaudet, M. M., Jemal, A., and Siegel, R. L. (2018). Ovarian cancer statistics, 2018. CA Cancer J Clin 68, 284-296.
Turk, H. F., and Chapkin, R. S. (2015). Analysis of epidermal growth factor receptor dimerization by BS (3) cross-linking. Methods Mol Biol 1233, 25-34.
Yoshikawa, T., Miyamoto, M., Aoyama, T., Soyama, H., Goto, T., Hirata, J., Suzuki, A., Nagaoka, I., Tsuda, H., Furuya, K., and Takano, M. (2018). JAK2/STAT3 pathway as a therapeutic target in ovarian cancers. Oncol Lett 15, 5772-5780.
Ziegenhain, C., Vieth, B., Parekh, S., Reinius, B., Guillaumet-Adkins, A., Smets, M., Leonhardt, H., Heyn, H., Hellmann, I., and Enard, W. (2017). Comparative Analysis of Single-Cell RNA Sequencing Methods. Mol Cell 65, 631-643 e634.
Zou, S., Tong, Q., Liu, B., Huang, W., Tian, Y., and Fu, X. (2020). Targeting STAT3 in Cancer Immunotherapy. Mol Cancer 19, 145.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. Various features and aspects of the present disclosure are set forth in the following claims.

1. An isolated monoclonal antibody, wherein the antibody specifically binds to OSMR and wherein the antibody competes for binding of the OSMR epitopes with an B01, B02, B03, B04, B05, B06, B07, B08, B09, B010, B12, B13, B14, B16, B17, B18, B19, B21, H09, H10, H11, H12, H13, H14, H15, or H16 monoclonal antibody.

2. The antibody of claim 1, or antigen-binding fragment thereof, wherein the antibody comprises:
(a) a first V_{H} CDR comprising V_{H} CDR1 of B01 (SEQ ID NO: 1), B02 (SEQ ID NO: 4), B03 (SEQ ID NO: 7), B04 (SEQ ID NO: 10), B05 (SEQ ID NO: 13), B06 (SEQ ID NO: 16), B07 (SEQ ID NO: 19), B08 (SEQ ID NO: 22), B09 (SEQ ID NO: 25), B10 (SEQ ID NO: 28), B12 (SEQ ID NO: 31), B13 (SEQ ID NO: 34), B14 (SEQ ID NO: 37), B16 (SEQ ID NO: 40), B17 (SEQ ID NO: 43), B18 (SEQ ID NO: 46), B19 (SEQ ID NO: 49), B21 (SEQ ID NO: 52), H09 (SEQ ID NO: 55), H10 (SEQ ID NO: 58), H11 (SEQ ID NO: 61), H12 (SEQ ID NO: 64), H13 (SEQ ID NO: 67), H14 (SEQ ID NO: 70), H15 (SEQ ID NO: 73), or H16 (SEQ ID NO: 76);
(b) a second V_{H} CDR comprising V_{H} CDR2 of B01 (SEQ ID NO: 2), B02 (SEQ ID NO: 5), B03 (SEQ ID NO: 8), B04 (SEQ ID NO: 11), B05 (SEQ ID NO: 14), B06 (SEQ ID NO: 17), B07 (SEQ ID NO: 20), B08 (SEQ ID NO: 23), B09 (SEQ ID NO: 26), B10 (SEQ ID NO: 29), B12 (SEQ ID NO: 32), B13 (SEQ ID NO: 35), B14 (SEQ ID NO: 38), B16 (SEQ ID NO: 41), B17 (SEQ ID NO: 44), B18 (SEQ ID NO: 47), B19 (SEQ ID NO: 50), B21 (SEQ ID NO: 53), H09 (SEQ ID NO: 56), H10 (SEQ ID NO: 59), H11 (SEQ ID NO: 62), H12 (SEQ ID NO: 65), H13 (SEQ ID NO: 68), H14 (SEQ ID NO: 71), H15 (SEQ ID NO: 74), or H16 (SEQ ID NO: 77);
(c) a third V_{H} CDR comprising V_{H} CDR3 of B01 (SEQ ID NO: 3), B02 (SEQ ID NO: 6), B03 (SEQ ID NO: 9), B04 (SEQ ID NO: 12), B05 (SEQ ID NO: 15), B06 (SEQ ID NO: 18), B07 (SEQ ID NO: 21), B08 (SEQ ID NO: 24), B09 (SEQ ID NO: 27), B10 (SEQ ID NO: 30), B12 (SEQ ID NO: 33), B13 (SEQ ID NO: 36), B14 (SEQ ID NO: 39), B16 (SEQ ID NO: 42), B17 (SEQ ID NO: 45), B18 (SEQ ID NO: 48), B19 (SEQ ID NO: 51), B21 (SEQ ID NO: 54), H09 (SEQ ID NO: 57), H10 (SEQ ID NO: 60), H11 (SEQ ID NO: 63), H12 (SEQ ID NO: 66), H13 (SEQ ID NO: 69), H14 (SEQ ID NO: 72), H15 (SEQ ID NO: 75), or H16 (SEQ ID NO: 78);
(d) a first V_{L} CDR comprising V_{L} CDR1 of B01 (SEQ ID NO: 79), B02 (SEQ ID NO: 81), B03 (SEQ ID NO: 83), B04 (SEQ ID NO: 85), B05 (SEQ ID NO: 87), B06 (SEQ ID NO: 89), B07 (SEQ ID NO: 91), B08 (SEQ ID NO: 93), B09 (SEQ ID NO: 95), B10 (SEQ ID NO: 97), B12 (SEQ ID NO: 99), B13 (SEQ ID NO: 101), B14 (SEQ ID NO: 103), B16 (SEQ ID NO: 105), B17 (SEQ ID NO: 107), B18 (SEQ ID NO: 109), B19 (SEQ ID NO: 111), B21 (SEQ ID NO: 113), H09 (SEQ ID NO: 115), H10 (SEQ ID NO: 117), H11 (SEQ ID NO: 119), H12 (SEQ ID NO: 121), H13 (SEQ ID NO: 123), H14 (SEQ ID NO: 125), H15 (SEQ ID NO: 127), or H16 (SEQ ID NO: 129);
(e) a second V_{L} CDR comprising V_{L} CDR2 of a tripeptide consisting of GNS, DNN, DAS, SHN, DAT, SNN, NNN, RNN, EDN, AAS, DVS, LGS, QDN, WDS, or PDC; and
(f) a third V_{L} CDR comprising V_{L} CDR3 of B01 (SEQ ID NO: 80), B02 (SEQ ID NO: 82), B03 (SEQ ID NO: 84), B04 (SEQ ID NO: 86), B05 (SEQ ID NO: 88), B06 (SEQ ID NO: 90), B07 (SEQ ID NO: 92), B08 (SEQ ID NO: 94), B09 (SEQ ID NO: 96), B10 (SEQ ID NO: 98), B12 (SEQ ID NO: 100), B13 (SEQ ID NO: 101), B14 (SEQ ID NO: 104), B16 (SEQ ID NO: 106), B17 (SEQ ID NO: 108), B18 (SEQ ID NO: 110), B19 (SEQ ID NO: 112), B21 (SEQ ID NO: 114), H09 (SEQ ID NO: 116), H10 (SEQ ID NO: 118), H11 (SEQ ID NO: 120), H12 (SEQ ID NO: 122), H13 (SEQ ID NO: 124), H14 (SEQ ID NO: 126), H15 (SEQ ID NO: 128), or H16 (SEQ ID NO: 130).

3-28. (canceled)

29. The antibody of claim 2, wherein the antibody comprises:
(i) a V_{H} domain at least about 80% identical to the V_{H} domain of B01 (SEQ ID NO: 131) or the humanized V_{H} domain of B01 mAb; and a V_{L} domain at least about 80% identical to the V_{L} domain of B01 (SEQ ID NO: 157) or the humanized V_{L} domain of B01 mAb;
(ii) a V_{H} domain at least about 80% identical to the V_{H} domain of B02 (SEQ ID NO: 132) or the humanized V_{H} domain of B02 mAb; and a V_{L} domain at least about 80% identical to the V_{L} domain of B02 (SEQ ID NO: 158) or the humanized V_{L} domain of B02 mA;
(iii) a V_{H} domain at least about 80% identical to the V_{H} domain of B03 (SEQ ID NO: 133) or the humanized V_{H} domain of B03 mAb; and a V_{L} domain at least about 80% identical to the V_{L} domain of B03 (SEQ ID NO: 159) or the humanized V_{L} domain of B03 mAb;
(iv) a V_{H} domain at least about 80% identical to the V_{H} domain of B04 (SEQ ID NO: 134) or the humanized V_{H} domain of B04 mAb; and a V_{L} domain at least about 80% identical to the V_{L} domain of B04 (SEQ ID NO: 160) or the humanized V_{L} domain of E1-80 mAb;
(v) a V_{H} domain at least about 80% identical to the V_{H} domain of B05 (SEQ ID NO: 135) or the humanized V_{H} domain of B05 mAb; and a V_{L} domain at least about 80% identical to the V_{L} domain of B05 (SEQ ID NO: 161) or the humanized V_{L} domain of B05 mAb;
(vi) a V_{H} domain at least about 80% identical to the V_{H} domain of B06 (SEQ ID NO: 136) or the humanized V_{H} domain of B06 mAb; and a V_{L} domain at least about 80% identical to the V_{L} domain of B06 (SEQ ID NO: 162) or the humanized V_{L} domain of B06 mAb;
(vii) a V_{H} domain at least about 80% identical to the V_{H} domain of B07 (SEQ ID NO: 137) or the humanized V_{H} domain of B07 mAb; and a V_{L} domain at least about 80% identical to the V_{L} domain of B07 (SEQ ID NO: 163) or the humanized V_{L} domain of B07 mAb;
(viii) a V_{H} domain at least about 80% identical to the V_{H} domain of B08 (SEQ ID NO: 138) or the humanized V_{H} domain of B08 mAb; and a V_{L} domain at least about 80% identical to the V_{L} domain of B08 (SEQ ID NO: 164) or the humanized V_{L} domain of B08 mAb;
(ix) a V_{H} domain at least about 80% identical to the V_{H} domain of B09 (SEQ ID NO: 139) or the humanized V_{H} domain of B09 mAb; and a V_{L} domain at least about 80% identical to the V_{L} domain of B09 (SEQ ID NO: 165) or the humanized V_{L} domain of B09 mAb;
(x) a V_{H} domain at least about 80% identical to the V_{H} domain of B10 (SEQ ID NO: 140) or the humanized V_{H} domain of B10 mAb; and a V_{L} domain at least about 80% identical to the V_{L} domain of B10 (SEQ ID NO: 166) or the humanized V_{L} domain of B10 mAb;
(xi) a V_{H} domain at least about 80% identical to the V_{H} domain of B12 (SEQ ID NO: 141) or the humanized V_{H} domain of B12 mAb; and a V_{L} domain at least about 80% identical to the V_{L} domain of B12 (SEQ ID NO: 167) or the humanized V_{L} domain of B12 mAb;
(xii) a V_{H} domain at least about 80% identical to the V_{H} domain of B13 (SEQ ID NO: 142) or the humanized V_{H} domain of B13 mAb; and a V_{L} domain at least about 80% identical to the V_{L} domain of B13 (SEQ ID NO: 168) or the humanized V_{L} domain of B13 mAb,
(xiii) a V_{H} domain at least about 80% identical to the V_{H} domain of B14 (SEQ ID NO: 143) or the humanized V_{H} domain of B14 mAb; and a V_{L} domain at least about 80% identical to the V_{L} domain of B14 (SEQ ID NO: 169) or the humanized V_{L} domain of B14 mAb,
(xiv) a V_{H} domain at least about 80% identical to the V_{H} domain of B16 (SEQ ID NO: 144) or the humanized V_{H} domain of B16 mAb; and a V_{L} domain at least about 80% identical to the V_{L} domain of B16 (SEQ ID NO: 170) or the humanized V_{L} domain of B16 mAb,
(xv) a V_{H} domain at least about 80% identical to the V_{H} domain of B17 (SEQ ID NO: 145) or the humanized V_{H} domain of B17 mAb; and a V_{L} domain at least about 80% identical to the V_{L} domain of B17 (SEQ ID NO: 171) or the humanized V_{L} domain of B17 mAb,
(xvi) a V_{H} domain at least about 80% identical to the V_{H} domain of B18 (SEQ ID NO: 146) or the humanized V_{H} domain of B18 mAb; and a V_{L} domain at least about 80% identical to the V_{L} domain of B18 (SEQ ID NO: 172) or the humanized V_{L} domain of B18 mAb,
(xvii) a V_{H} domain at least about 80% identical to the V_{H} domain of B19 (SEQ ID NO: 147) or the humanized V_{H} domain of B19 mAb; and a V_{L} domain at least about 80% identical to the V_{L} domain of B19 (SEQ ID NO: 173) or the humanized V_{L} domain of B19 mAb,
(xviii) a V_{H} domain at least about 80% identical to the V_{H} domain of B21 (SEQ ID NO: 148) or the humanized V_{H} domain of B21 mAb; and a V_{L} domain at least about 80% identical to the V_{L} domain of B21 (SEQ ID NO: 174) or the humanized V_{L} domain of B21 mAb,
(xix) a V_{H} domain at least about 80% identical to the V_{H} domain of H09 (SEQ ID NO: 149) or the humanized V_{H} domain of H09 mAb; and a V_{L} domain at least about 80% identical to the V_{L} domain of H09 (SEQ ID NO: 175) or the humanized V_{L} domain of H09 mAb,
(xx) a V_{H} domain at least about 80% identical to the V_{H} domain of H10 (SEQ ID NO: 150) or the humanized V_{H} domain of H10 mAb; and a V_{L} domain at least about 80% identical to the V_{L} domain of H10 (SEQ ID NO: 176) or the humanized V_{L} domain of H10 mAb,
(xxi) a V_{H} domain at least about 80% identical to the V_{H} domain of H11 (SEQ ID NO: 151) or the humanized V_{H} domain of H11 mAb; and a V_{L} domain at least about 80% identical to the V_{L} domain of H11 (SEQ ID NO: 177) or the humanized V_{L} domain of H11 mAb,
(xxii) a V_{H} domain at least about 80% identical to the V_{H} domain of H12 (SEQ ID NO: 152) or the humanized V_{H} domain of H12 mAb; and a V_{L} domain at least about 80% identical to the V_{L} domain of H12 (SEQ ID NO: 178) or the humanized V_{L} domain of H12 mAb,
(xxiii) a V_{H} domain at least about 80% identical to the V_{H} domain of H13 (SEQ ID NO: 153) or the humanized V_{H} domain of H13 mAb; and a V_{L} domain at least about 80% identical to the V_{L} domain of H13 (SEQ ID NO: 179) or the humanized V_{L} domain of H13 mAb,
(xxiv) a V_{H} domain at least about 80% identical to the V_{H} domain of H14 (SEQ ID NO: 154) or the humanized V_{H} domain of H14 mAb; and a V_{L} domain at least about 80% identical to the V_{L} domain of H14 (SEQ ID NO: 180) or the humanized V_{L} domain of H14 mAb,
(xxv) a V_{H} domain at least about 80% identical to the V_{H} domain of H15 (SEQ ID NO: 155) or the humanized V_{H} domain of H15 mAb; and a V_{L} domain at least about 80% identical to the V_{L} domain of H15 (SEQ ID NO: 181) or the humanized V_{L} domain of H15 mAb,
(xxvi) a V_{H} domain at least about 80% identical to the V_{H} domain of H16 (SEQ ID NO: 156) or the humanized V_{H} domain of H16 mAb; and a V_{L} domain at least about 80% identical to the V_{L} domain of H16 (SEQ ID NO: 182) or the humanized V_{L} domain of H16 mAb.

30-55. (canceled)

56. The antibody of claim 29, wherein the antibody is the B01, B02, B03, B04, B05, B06, B07, B08, B09, B010, B12, B13, B14, B16, B17, B18, B19, B21, H09, H10, H11, H12, H13, H14, H15, or H16 antibody.

57. The antibody of claim 1, wherein the antibody is recombinant.

58. The antibody of claim 1, wherein the antibody is an IgG, IgM, IgA or an antigen binding fragment thereof.

59. The antibody of claim 1, wherein the antibody is a Fab′, a F(ab′)2, a F(ab′)3, a monovalent scFv, a bivalent scFv, or a single domain antibody.

60. The antibody of claim 1, wherein the antibody is a human, humanized antibody or de-immunized antibody.

61. The antibody of claim 1, wherein the antibody is conjugated to an imaging agent, a chemotherapeutic agent, a toxin or a radionuclide.

62. The antibody of claim 61, wherein the antibody is conjugated to a toxin.

63. The antibody of claim 62, wherein the toxin is auristatin or monomethyl auristatin E (MMAE).

64. (canceled)

65. A chimeric antigen receptor comprising an antigen-binding domain at least 80% identical to an antigen-binding domain of the monoclonal antibody of claim 1.

66. A composition comprising an antibody of claim 1 in a pharmaceutically acceptable carrier.

67. An isolated polynucleotide molecule comprising a nucleic acid sequence encoding an antibody of claim 1.

68. A recombinant polypeptide comprising an antibody V_{H} domain comprising CDRs 1-3 of the V_{H} domain of B01 (SEQ ID NOs: 1, 2, and 3); CDRs 1-3 of the V_{H} domain of B02 (SEQ ID NOs: 4, 5, and 6); CDRs 1-3 of the V_{H} domain of B03 (SEQ ID NOs: 7, 8, and 9); CDRs 1-3 of the V_{H} domain of B04 (SEQ ID NOs: 10, 11, and 12); CDRs 1-3 of the V_{H} domain of B05 (SEQ ID NOs: 13, 14, and 15); CDRs 1-3 of the V_{H} domain of B06 (SEQ ID NOs: 16, 17, and 18); CDRs 1-3 of the V_{H} domain of B07 (SEQ ID NOs: 19, 20, and 21); CDRs 1-3 of the V_{H} domain of B08 (SEQ ID NOs: 22, 23, and 24); CDRs 1-3 of the V_{H} domain of B09 (SEQ ID NOs: 25, 26, and 27); CDRs 1-3 of the V_{H} domain of B10 (SEQ ID NOs: 28, 29, and 30); CDRs 1-3 of the V_{H} domain of B12 (SEQ ID NOs: 31, 32, and 33); CDRs 1-3 of the V_{H} domain of B13 (SEQ ID NOs: 34, 35, and 36); CDRs 1-3 of the V_{H} domain of B14 (SEQ ID NOs: 37, 38, and 39); CDRs 1-3 of the V_{H} domain of B16 (SEQ ID NOs: 40, 41, and 42); CDRs 1-3 of the V_{H} domain of B17 (SEQ ID NOs: 43, 44, and 45); CDRs 1-3 of the V_{H} domain of B18 (SEQ ID NOs: 46, 47, and 48); CDRs 1-3 of the V_{H} domain of B19 (SEQ ID NOs: 49, 50, and 51); CDRs 1-3 of the V_{H} domain of B21 (SEQ ID NOs: 52, 53, and 54); CDRs 1-3 of the V_{H} domain of H09 (SEQ ID NOs: 55, 56, and 57); CDRs 1-3 of the V_{H} domain of H10 (SEQ ID NOs: 58, 59, 60); CDRs 1-3 of the V_{H} domain of H11 (SEQ ID NOs: 61, 62, and 63); CDRs 1-3 of the V_{H} domain of H12 (SEQ ID NOs: 64, 65, and 66); CDRs 1-3 of the V_{H} domain of H13 (SEQ ID NOs: 67, 68, and 69); CDRs 1-3 of the Vu domain of H14 (SEQ ID NOs: 70, 71, and 72); CDRs 1-3 of the V_{H} domain of B15 (SEQ ID NOs: 73, 74, and 75); or CDRs 1-3 of the V_{H} domain of B16 (SEQ ID NOs: 76, 77, and 78).

69. A recombinant polypeptide comprising an antibody V_{L} domain comprising CDRs 1-3 of the V_{L} domain of B01 (SEQ ID NO: 79, the tripeptide GNS, and SEQ ID NO: 80); CDRs 1-3 of the V_{L} domain of B02 (SEQ ID NO: 81, the tripeptide DNN, and SEQ ID NO: 82); CDRs 1-3 of the V_{L} domain of B03 (SEQ ID NO: 83, the tripeptide DAS, and SEQ ID NO: 84); CDRs 1-3 of the V_{L} domain of B04 (SEQ ID NO: 85, the tripeptide DAS, and SEQ ID NO: 86); CDRs 1-3 of the V_{L} domain of B05 (SEQ ID NO: 87, the tripeptide DAS, and SEQ ID NO: 88); CDRs 1-3 of the V_{L} domain of B06 (SEQ ID NO: 89, the tripeptide SHN, and SEQ ID NO: 90); CDRs 1-3 of the V_{L} domain of B07 (SEQ ID NO: 91, the tripeptide DAT, and SEQ ID NO: 92); CDRs 1-3 of the V_{L} domain of B08 (SEQ ID NO: 93, the tripeptide SNN, and SEQ ID NO: 94); CDRs 1-3 of the V_{L} domain of B09 (SEQ ID NO: 95, the tripeptide NNN, and SEQ ID NO: 96); CDRs 1-3 of the V_{L} domain of B10 (SEQ ID NO: 97, the tripeptide RNN, and SEQ ID NO: 98); CDRs 1-3 of the V_{L} domain of B12 (SEQ ID NO: 99, the tripeptide EDN, and SEQ ID NO: 100); CDRs 1-3 of the V_{L} domain of B13 (SEQ ID NO: 101, the tripeptide SNN, and SEQ ID NO: 102); CDRs 1-3 of the V_{L} domain of B14 (SEQ ID NO: 103, the tripeptide AAS, and SEQ ID NO: 104); CDRs 1-3 of the V_{L} domain of B16 (SEQ ID NO: 105, the tripeptide DAS, and SEQ ID NO: 106); CDRs 1-3 of the V_{L} domain of B17 (SEQ ID NO: 107, the tripeptide DVS, and SEQ ID NO: 108); CDRs 1-3 of the V_{L} domain of B18 (SEQ ID NO: 109, the tripeptide LGS, and SEQ ID NO: 110); CDRs 1-3 of the V_{L} domain of B19 (SEQ ID NO: 111, the tripeptide SNN, and SEQ ID NO: 112); CDRs 1-3 of the V_{L} domain of B21 (SEQ ID NO: 113, the tripeptide AAS, and SEQ ID NO: 114); CDRs 1-3 of the V_{L} domain of H09 (SEQ ID NO: 115, the tripeptide SNN, and SEQ ID NO: 116); CDRs 1-3 of the V_{L} domain of H10 (SEQ ID NO: 117, the tripeptide SNN, and SEQ ID NO: 118); CDRs 1-3 of the V_{L} domain of H11 (SEQ ID NO: 119, the tripeptide QDN, and SEQ ID NO: 120); CDRs 1-3 of the V_{L} domain of H12 (SEQ ID NO: 121, the tripeptide WDS, and SEQ ID NO: 122); CDRs 1-3 of the V_{L} domain of H13 (SEQ ID NO: 123, the tripeptide EDN, and SEQ ID NO: 124); CDRs 1-3 of the V_{L} domain of H14 (SEQ ID NO: 125, the tripeptide SNN, and SEQ ID NO: 126); CDRs 1-3 of the V_{L} domain of H15 (SEQ ID NO: 127, the tripeptide PDC, and SEQ ID NO: 128); or CDRs 1-3 of the V_{L} domain of H16 (SEQ ID NO: 129, the tripeptide AAS, and SEQ ID NO: 130).

70. An isolated polynucleotide molecule comprising a nucleic acid sequence encoding a polypeptide of claim 68.

71. A host cell comprising one or more polynucleotide molecule(s) encoding an antibody of claim 1.

72. (canceled)

73. A method of manufacturing an antibody comprising:
(a) expressing one or more polynucleotide molecule(s) encoding a V_{L} and V_{H} chain of an antibody of claim 1 in a cell; and
(b) purifying the antibody from the cell.

74. A method for treating a subject having a cancer comprising administering an effective amount of an antibody of claim 1 to the subject.

75. The method of claim 74, wherein the cancer is a breast cancer, lung cancer, head & neck cancer, prostate cancer, esophageal cancer, tracheal cancer, skin cancer brain cancer, liver cancer, bladder cancer, stomach cancer, pancreatic cancer, epithelial cancer, uterine cancer, cervical cancer, testicular cancer, colon cancer, rectal cancer or skin cancer.

76-83. (canceled)

84. The methods of claim 83, wherein the chemotherapy is a chosen from carboplatin (or cisplatin), a taxane, such as paclitaxel (Taxol®) or docetaxel (Taxotere®), bevacizumab (Alymsys®, Avastin®, Mvasi®, Zirabev®), and a poly ADP ribose polymerase (PARP) Inhibitor (such as but not limited to, niraparib (Zejula®) rucaparib (Rubraca®) and olaparib (Lynparza®)), or a combination thereof.
